(19) United States
(12) Patent Application Publication
(10) Pub. No.: US 2012/0283228 A1
(43) Pub. Date: Nov. 8, 2012

(54) 2alpha-Methyl and 2beta-Methyl Analogs of 19,26-Dinor-1alpha,25-Dihydroxyvitamin D3 and Their Uses

(75) Inventors: Hector F. DeLuca, Deerfield, WI (US); Margaret Clagett-Dame, Deerfield, WI (US); Pawel Grzywacz, Madison, WI (US); Grazia Chiellini, Madison, WI (US); Lori A. Plum, Arena, WI (US)

(73) Assignee: WISCONSIN ALUMNI RESEARCH FOUNDATION, Madison, WI (US)

(21) Appl. No.: 13/463,557

(22) Filed: May 3, 2012

Related U.S. Application Data
(60) Provisional application No. 61/482,007, filed on May 3, 2011.

(51) Int. Cl. C07C 401/00; A61P 17/06; A61P 35/00; A61P 35/02; A61P 17/00; A61P 3/10; A61P 29/00; A61P 11/06; A61P 1/00; A61K 31/593; A61P 37/06

(52) U.S. Cl. 514/167; 552/653

(57) ABSTRACT

This invention discloses 2α-methyl and 2β-methyl analogs of 19,26-dinor-1α,25-dihydroxyvitamin D₃ and pharmaceutical uses therefor. These compounds exhibit in vitro biological activities evidencing use as an anti-cancer agent and for the treatment of skin diseases such as psoriasis as well as skin conditions such as wrinkles, slack skin, dry skin and insufficient sebum secretion. These compounds have little, if any, in vivo calcemic activity and therefore may be used to treat autoimmune disorders in humans as well as secondary hyperparathyroidism and renal osteodystrophy.

### CROSS REFERENCE TO RELATED APPLICATIONS

This application claims the benefit of U.S. Provisional Application No. 61/482,007, filed May 3, 2011, which is incorporated by reference herein in its entirety for any purpose.

### BACKGROUND OF THE INVENTION

This invention relates to vitamin D compounds, and more particularly to 2α-methyl and 2β-methyl analogs of 19,26-dinor-1α,25-dihydroxyvitamin D₃ and their pharmaceutical uses.

The natural hormone, 1α,25-dihydroxyvitamin D₃ and its analog in ergosterol series, i.e. 1α,25-dihydroxyvitamin D₂ are known to be highly potent regulators of calcium homeostasis in animals and humans, and their activity in cellular differentiation has also been established, Ostrem et al., Proc. Natl. Acad. Sci. USA, 84, 2610 (1987). Many structural analogs of these metabolites have been prepared and tested, including 1α-hydroxyvitamin D₃, 1α-hydroxyvitamin D₂, various side chain homologated vitamins and fluorinated analogs. Some of these compounds exhibit an interesting separation of activities in cell differentiation and calcium regulation. This difference in activity may be useful in the treatment of a variety of diseases such as renal osteodystrophy, vitamin D-resistant rickets, osteoporosis, psoriasis, and certain malignancies.

Another class of vitamin D analogs, i.e. the so called 19-nor-vitamin D compounds, is characterized by the replacement of the A-ring exocyclic methylene group (carbon 19), typical of the vitamin D system, by two hydrogen atoms. Biological testing of such 19-nor-analogs (e.g., 1α,25-dihydroxy-19-nor-vitamin D₃) revealed a selective activity profile with high potency in inducing cellular differentiation, and very low calcium mobilizing activity. Thus these compounds are potentially useful as therapeutic agents for the treatment of malignancies, or the treatment of various skin disorders. Two different methods of synthesis of such 19-nor-vitamin D analogs have been described (Perlman et al., Tetrahedron Lett. 31, 1823 (1990); Perlman et al., Tetrahedron Lett. 32, 7663(1991), and DeLuca et al., U.S. Pat. No. 5,086,191).

In U.S. Pat. No. 4,666,634, 2β-hydroxy and alkoxy (e.g., ED-71) analogs of 1α,25-dihydroxyvitamin D₃ have been described and examined by Chugai group as potential drugs for osteoporosis and as antitumor agents. See also Okano et al., Biochem. Biophys. Res. Commun. 163, 1444 (1989). Other 2-substituted (with hydroxyalykl, e.g., ED-120, and fluoroalkyl groups) A-ring analogs of 1α,25-dihydroxyvitamin D₃ have also been prepared and tested (Miyamoto et al., Chem. Pharm. Bull. 41, 1111 (1993); Nishii et al., Osteoporosis Int. Suppl. 1, 190 (1993); Posner et al., J. Org. Chem. 5, 7855 (1994), and J. Org. Chem. 60, 4617 (1995)).

2-Substituted analogs of 1α,25-dihydroxy-19-nor-vitamin D₃ have also been synthesized, i.e. compounds substituted at 2-position with hydroxy or alkoxy groups (DeLuca et al., U.S. Pat. No. 5,536,713), with 2-alkyl groups (DeLuca et al U.S. Pat. No. 5,945,410), and with 2-alkylidene groups (DeLuca et al U.S. Pat. No. 5,843,928), which exhibit interesting and selective activity profiles. All these studies indicate that binding sites in vitamin D receptors can accommodate different substituents at C-2 in the synthesized vitamin D analogs.

In a continuing effort to explore the 19-nor class of pharmacologically important vitamin D compounds, analogs which are characterized by the presence of a methylene substituent at carbon 2 (C-2), a hydroxyl group at carbon 1 (C-1), and a shortened side chain attached to carbon 20 (C-20) have also been synthesized and tested. 1α-Hydroxy-2-methylene-19-nor-pregnacalciferol is described in U.S. Pat. No. 6,566,352 while 1α-hydroxy-2-methylene-19-nor-homopregnacalciferol is described in U.S. Pat. No. 6,579,861 and 1α-hydroxy-2-methylene-19-nor-bishomopregnacalciferol is described in U.S. Pat. No. 6,627,622. All three of these compounds have relatively high binding activity to vitamin D receptors and relatively high cell differentiation activity, but little if any calcemic activity as compared to 1α,25-dihydroxyvitamin D₃. Their biological activities make these compounds excellent candidates for a variety of pharmaceutical uses, as set forth in the '352, '861 and '622 patents.

### SUMMARY OF THE INVENTION

The present invention is directed toward 2α-methyl and 2β-methyl analogs of 19,26-dinor-1α,25-dihydroxyvitamin D₃, and particularly to the 2α-methyl and 2β-methyl analogs of 19,26-dinor-1α,25-dihydroxyvitamin D₃ wherein either or both of the 20-methyl and 25-hydroxyl substituents of the analogs may be orientated independently in their R or S configurations. In other words, one of the 20-methyl or 25-hydroxyl substituents may be in its R orientation and the other in its S orientation, or both substituents may be in their R orientation or both in their S orientation. The biological activity, and various pharmaceutical uses for these compounds, are also described.

Structurally the 2α-methyl and 2β-methyl-19,26-dinor vitamin D₃ analogs are characterized by the general formula I shown below:

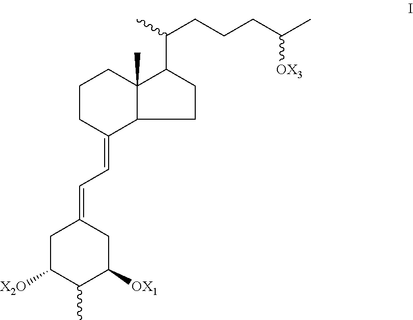

where the methyl group attached to carbon 2 of the A-ring is in either its R or S configuration, and where the methyl group attached to carbon 20 is in either its R or S configuration, and where the substituent —OX₃ is in either its R or S configuration, as indicated by the wavy lines in the above formula I, and where each of X₁, X₂, and X₃ which may be the same or different, is independently selected from hydrogen or a hydroxy-protecting group. The preferred analogs are 2α-methyl-19,26-dinor-1α,25-dihydroxyvitamin D₃ analogs which have the formula Ia:

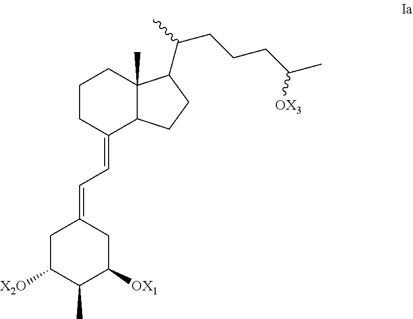

and 2β-methyl-19,26-dinor-1α,25-dihydroxyvitamin D₃ analogs, which have the formula Ib:

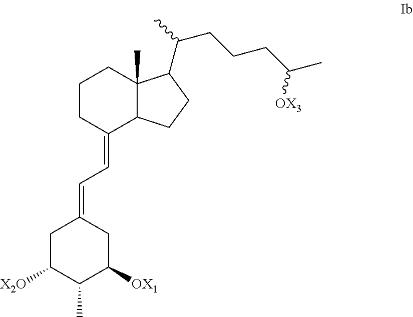

The above compounds exhibit desired, and highly advantageous, patterns of biological activity. With regard to in vitro activities, all of the above compounds exhibit, to different degrees depending on the analog, binding to vitamin D receptors, HL-60 cell differentiation, and 24-hydroxylase gene transactivation. With regard to in vivo calcemic activities, all of the above compounds have very low ability to mobilize calcium from bone, and have relatively insignificant intestinal calcium absorption activity, as compared to 1α,25-dihydroxyvitamin D₃. Hence, these compounds can be characterized as having little, if any, calcemic activity.

It is undesirable to raise serum calcium to supraphysiologic levels when suppressing the preproparathyroid hormone gene (Darwish & DeLuca, Arch. Biochem. Biophys. 365, 123-130, 1999) and parathyroid gland proliferation. These analogs having little or no calcemic activity while relatively active on cell differentiation are expected to be useful as a therapy for suppression of secondary hyperparathyroidism of renal osteodystrophy, and renal osteodystrophy per se.

The compounds of this invention have also been discovered to be especially suited for treatment and prophylaxis of human disorders which are characterized by an imbalance in the immune system, e.g. in autoimmune diseases including multiple sclerosis, lupus, diabetes mellitus, host versus graft rejection, and rejection of organ transplants; and additionally for the treatment of inflammatory diseases, such as rheumatoid arthritis, asthma, and inflammatory bowel diseases such as celiac disease, ulcerative colitis and Crohn's disease. Acne, alopecia and hypertension are other conditions which may be treated with the compound of the invention.

The above compounds are also characterized by relatively high cell differentiation activity. Thus, these compounds also provide a therapeutic agent for the treatment of psoriasis, or as an anti-cancer agent, especially against leukemia, colon cancer, breast cancer, skin cancer and prostate cancer. In addition, due to their relatively high cell differentiation activity, these compounds provide therapeutic agents for the treatment of various skin conditions including wrinkles, lack of adequate dermal hydration, i.e. dry skin, lack of adequate skin firmness, i.e. slack skin, and insufficient sebum secretion. Use of these compounds thus not only results in moisturizing of skin but also improves the barrier function of skin.

The compounds of the invention of formula I are also useful in preventing or treating obesity, inhibiting adipocyte differentiation, inhibiting SCD-1 gene transcription, and/or reducing body fat in animal subjects. Therefore, in some embodiments, a method of preventing or treating obesity, inhibiting adipocyte, differentiations inhibiting SCD-1 gene transcription, and/or reducing body fat in an animal subject includes administering to the animal subject, an effective amount of one or more of the compounds or a pharmaceutical composition that includes one or more of the compounds of formula I. Administration of the compound or the pharmaceutical compositions to the subject inhibits adipocyte differentiation, inhibits gene transcription, and/or reduces body fat in the animal subject.

The compounds may be present in a composition to treat the above-noted diseases and disorders in an amount from about 0.01 μg/gm to about 100 μg/gm of the composition, preferably from about 0.1 μg/gm to about 50 μg/gm of the composition, and may be administered topically, nasally, rectally, sublingually, transdermally, orally or parenterally in dosages of from about 0.01 μg/day to about 1000 μg/day, preferably from about 0.1 μg/day to about 500 μg/day.

### BRIEF DESCRIPTION OF THE DRAWINGS

FIGS. 1-40 illustrate various biological activities of the 2α-methyl and 2β-methyl analogs of 19,26-dinor-1α,25-dihydroxyvitamin D₃, as compared to the native hormone 1α,25-dihydroxyvitamin D₃, hereinafter “1,25(OH)₂D₃.”

FIGS. 1-5 illustrate the activities of (20S,25R)-2α-methyl-19,26-dinor-1α,25-dihydroxyvitamin D₃, hereinafter referred to as “LR-2.”

FIGS. 6-10 illustrate the activities of (20S,25R)-2β-dimethyl-19,26-dinor-1α,25-dihydroxyvitamin D₃, hereinafter referred to as “FD-1.”

FIGS. 11-15 illustrate the activities of (20R,25R)-2α-methyl-19,26-dinor-1α,25-dihydroxyvitamin D₃, hereinafter referred to as “MY-2.”

FIGS. 16-20 illustrate, the activities of (20R,25R)-2β-methyl-19,26-dinlor-1α,25-dihydroxyvitamin D₃, hereinafter referred to as “DW-1.”

FIGS. 21-25 illustrate the activities of (20S,25S)-2α-methyl-19,26-dinor-1α,25-dihydroxyvitamin D₃, hereinafter referred to as “TA-2.”

FIGS. 26-30 illustrate, the activities of (20S,25S)-2-methyl-19,26-dinor-1α,25-dihydroxyvitamin D₃, hereinafter referred to as “IB-1.”

FIGS. 31-35 illustrate the activities of (20R,25S)-2α-methyl-19,26-dinor-1α,25-dihydroxyvitamin D₃, hereinafter referred to as “NC-2.”

FIGS. 36-40 illustrate the activities of (20R,25S)-2β-methyl-19,26-dinor-1α,25-dihydroxyvitamin D₃, hereinafter referred to as “TH-1.”

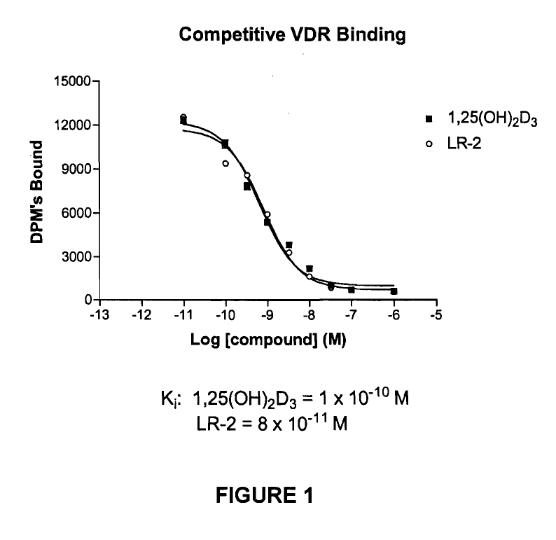

FIG. 1 is a graph illustrating the relative activity of LR-2 and 1,25(OH)₂D₃ to compete for binding with [³H]-1,25-(OH)₂-D₃ to the full-length recombinant rat vitamin D receptor;

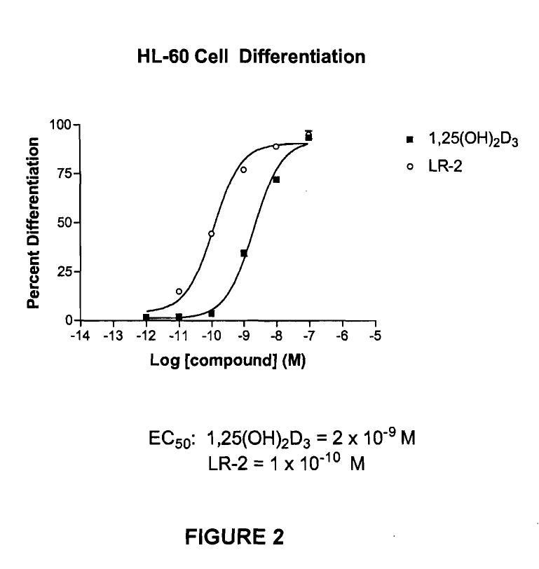

FIG. 2 is a graph illustrating the percent HL-60 cell differentiation as a function of the concentration of LR-2 and 1,25(OH)₂D₃;

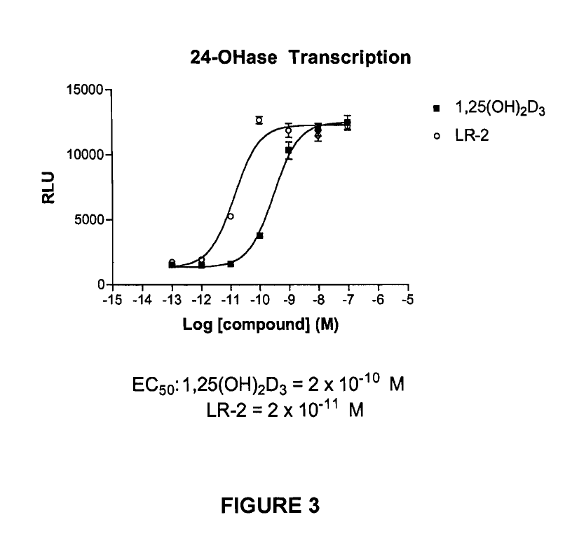

FIG. 3 is a bar graph illustrating the in vitro transcription activity of 1,25(OH)₂D₃ as compared to LR-2;

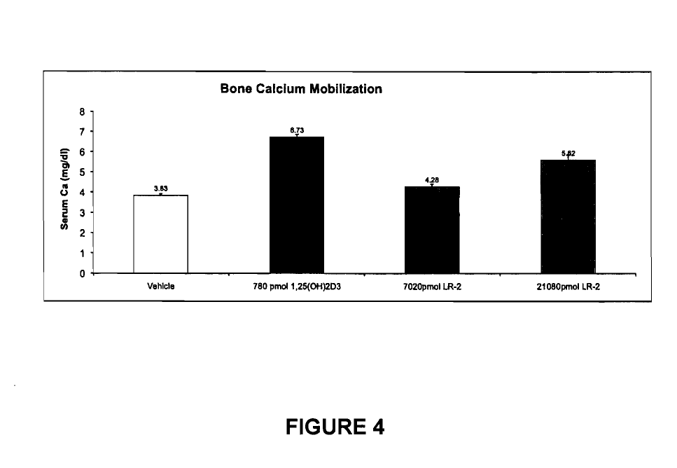

FIG. 4 is a bar graph illustrating the bone calcium mobilization activity of 1,25(OH)₂D₃ as compared to LR-2;

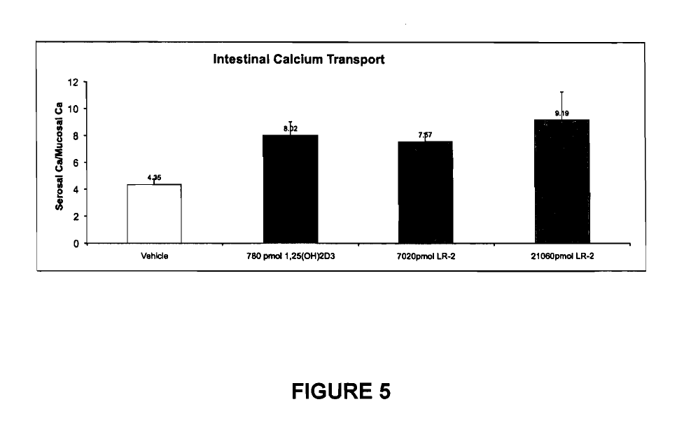

FIG. 5 is a bar graph illustrating the intestinal calcium transport activity of 1,25(OH)₂D₃ as compared to LR-2.

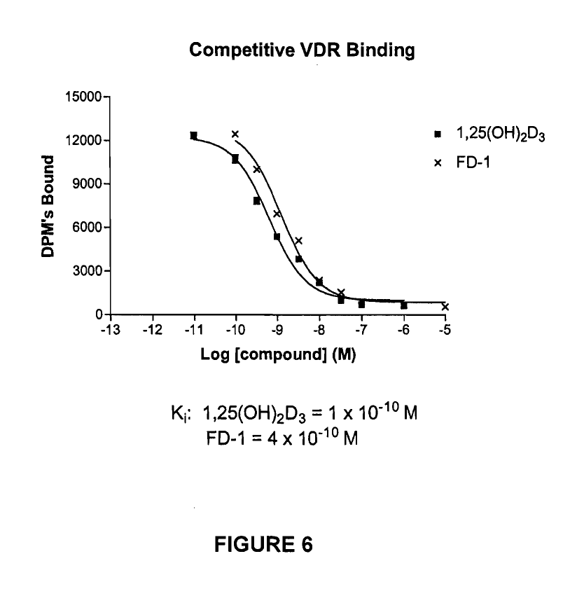

FIG. 6 is a graph illustrating the relative activity of FD-1 and 1,25(OH)₂D₃ to compete for binding with [³H]-1,25-(OH)₂-D₃ to the full-length recombinant rat vitamin D-receptor;

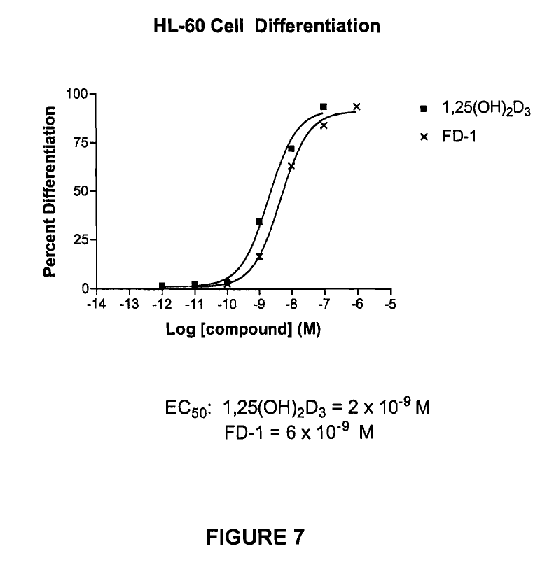

FIG. 7 is a graph illustrating the percent HL-60 cell differentiation as a function of the concentration of FD-1 and 1,25(OH)₂D₃;

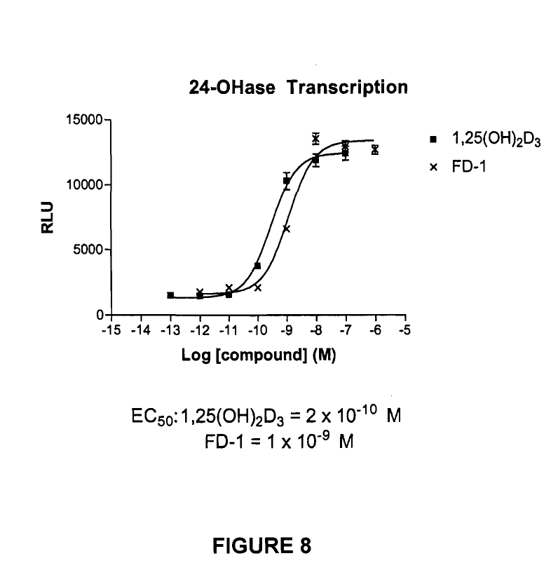

FIG. 8 is a bar graph illustrating the in vitro transcription activity of 1,25(OH)₂D₃ as compared to FD-1;

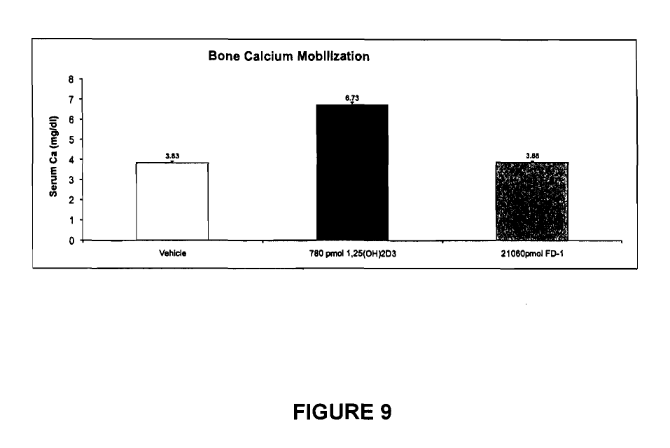

FIG. 9 is a bar graph illustrating the bone calcium mobilization activity of 1,25(OH)₂D₃ as compared to FD-1;

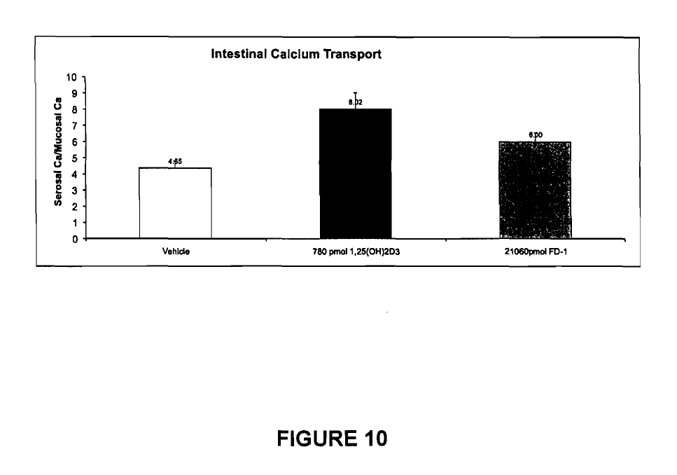

FIG. 10 is a bar graph illustrating the intestinal calcium transport activity of 1,25(OH)₂D₃ as compared to FD-1;

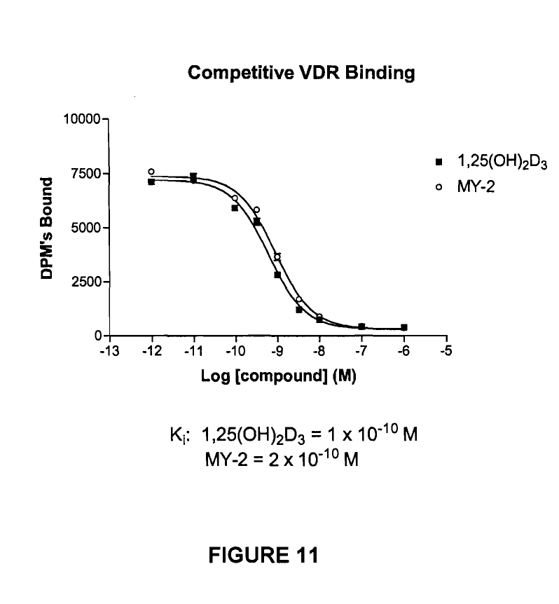

FIG. 11 is a graph illustrating the relative activity of MY-2 and 1,25(OH)₂D₃ to compete for binding with [³H]-1,25-(OH)₂-D₃ to the full-length recombinant rat vitamin-D receptor;

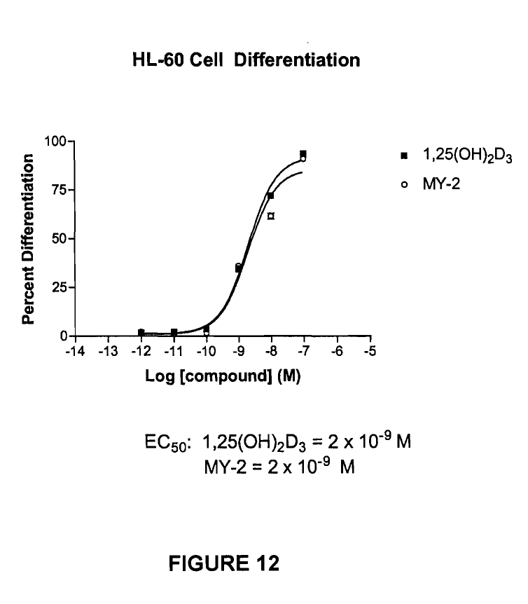

FIG. 12 is a graph illustrating the percent HL-60 cell differentiation, as a function of the concentration of MY-2 and 1,25(OH)₂D₃;

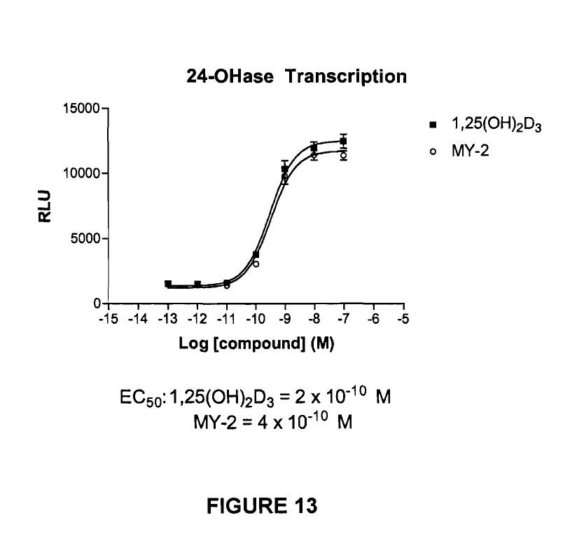

FIG. 13 is a bar graph illustrating the in vitro transcription activity of 1,25(OH)₂D₃ as compared to MY-2;

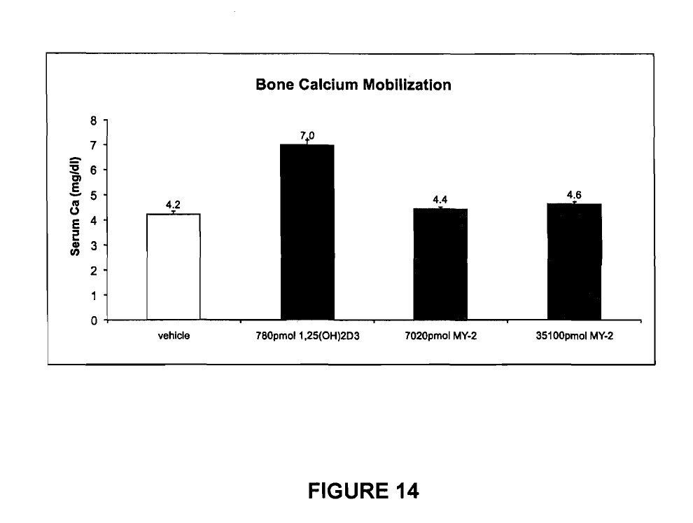

FIG. 14 is a bar graph illustrating the bone calcium mobilization activity of 1,25(OH)₂D₃ as compared to MY-2;

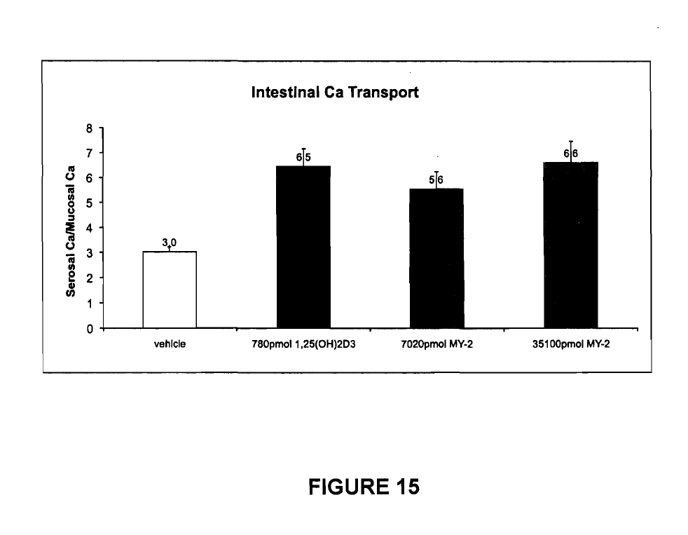

FIG. 15 is a bar graph illustrating the intestinal calcium transport activity of 1,25(OH)₂D₃ as compared to MY-2;

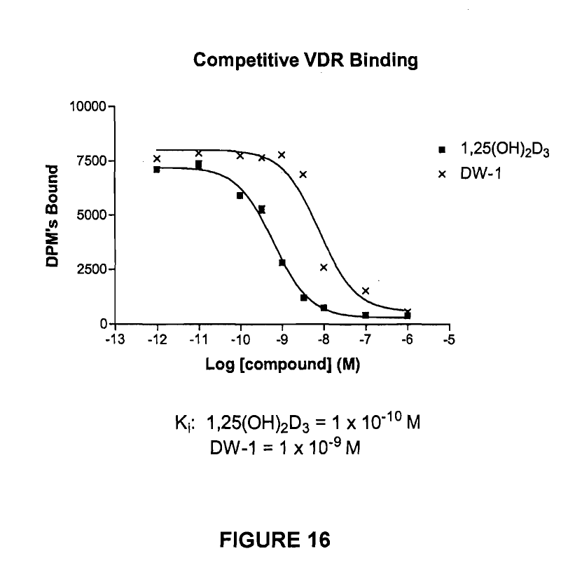

FIG. 16 is a graph illustrating the relative activity of DW-1 and 1,25(OH)₂D₃ to compete for binding with [³H]-1,25-(OH)₂-D₃ to the full length recombinant rat vitamin D receptor;

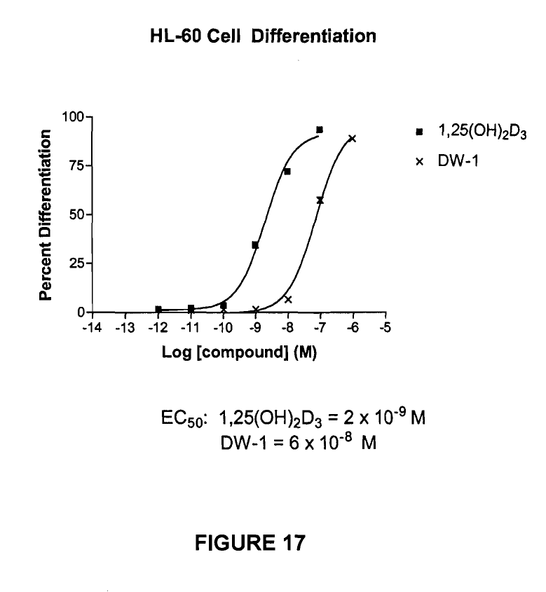

FIG. 17 is a graph illustrating the percent HL-60 cell differentiation as a function of the concentration of DW-1 and 1,25(OH)₂D₃;

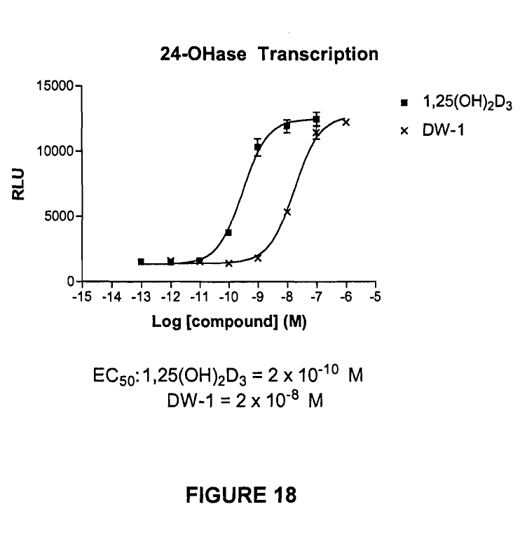

FIG. 18 is a bar graph illustrating the in vitro transcription activity of 1,25(OH)₂D₃ as compared to DW-1;

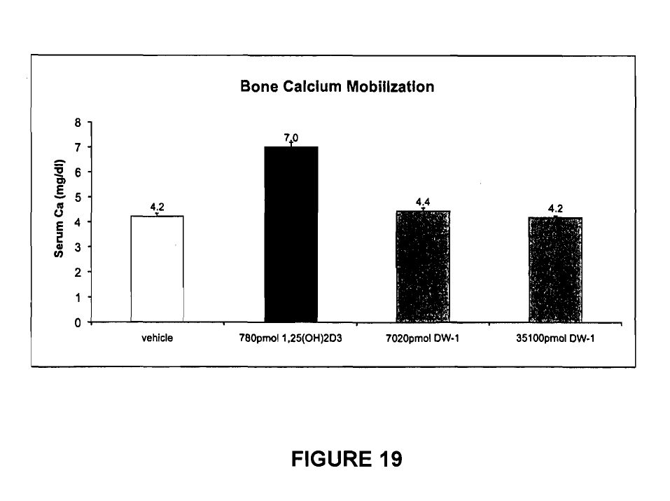

FIG. 19 is a bar graph illustrating the bone calcium mobilization activity of 1,25(OH)₂D₃ as compared to DW-1;

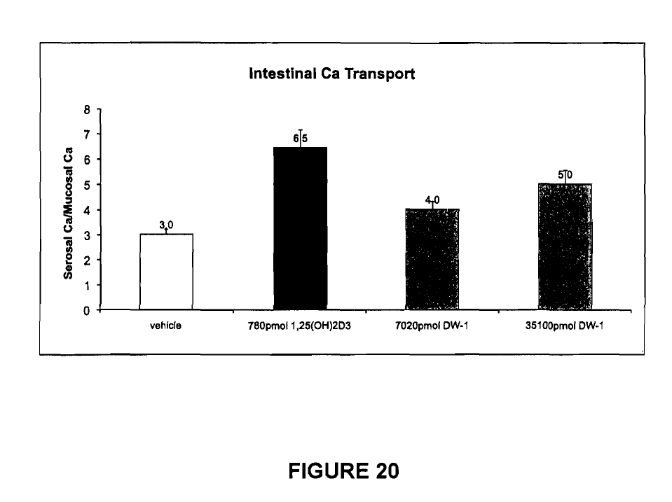

FIG. 20 is a bar graph illustrating the intestinal calcium transport activity of 1,25(OH)₂D₃ as compared to DW-1;

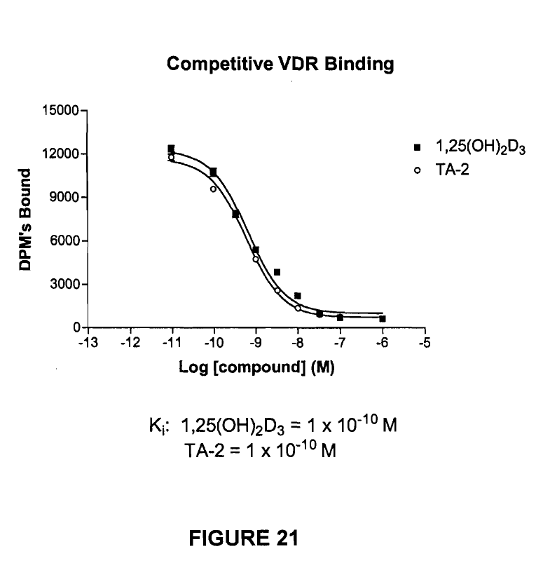

FIG. 21 is a graph illustrating the relative activity of TA-2 and 1,25(OH)₂D₃ to compete for binding with [³H]-1,25-(OH)₂-D₃ to the full-length recombinant rat vitamin D receptor;

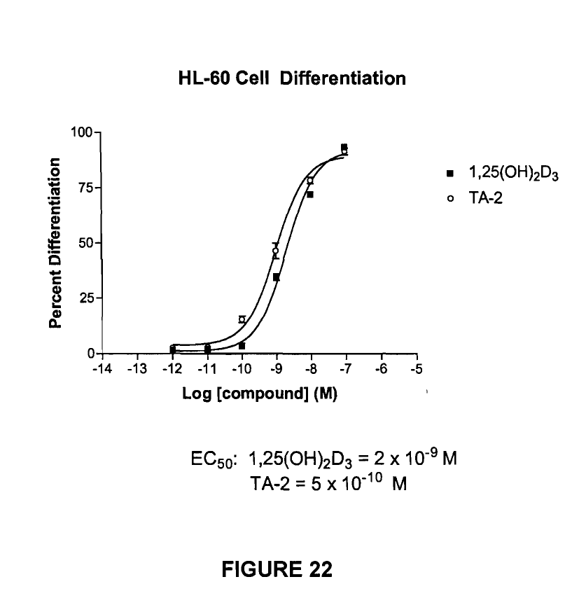

FIG. 22 is a graph illustrating the percent HL-60 cell differentiation as a function of the concentration of TA-2 and 1,25(OH)₂D₃;

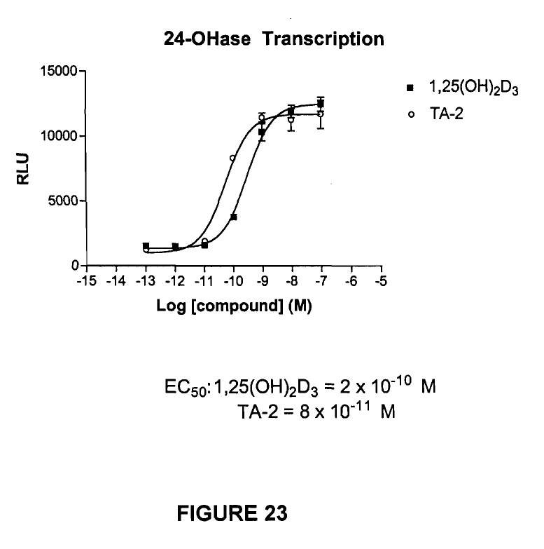

FIG. 23 is a bar graph illustrating the in vitro transcription activity of 1,25(OH)₂D₃ as compared to TA-2;

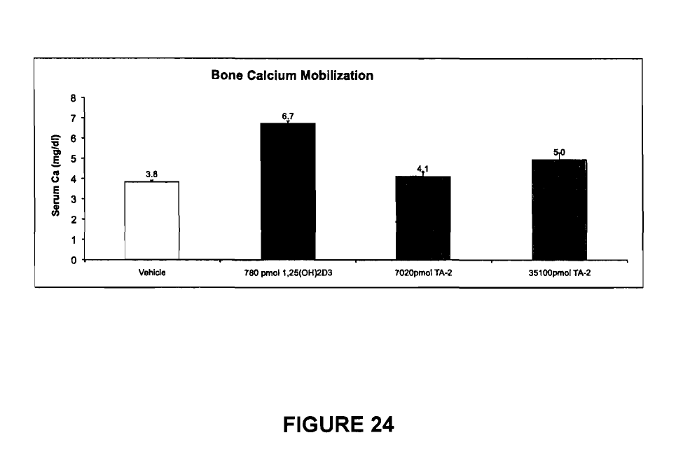

FIG. 24 is a bar graph illustrating the bone calcium mobilization activity of 1,25(OH)₂D₃ as compared to TA-2;

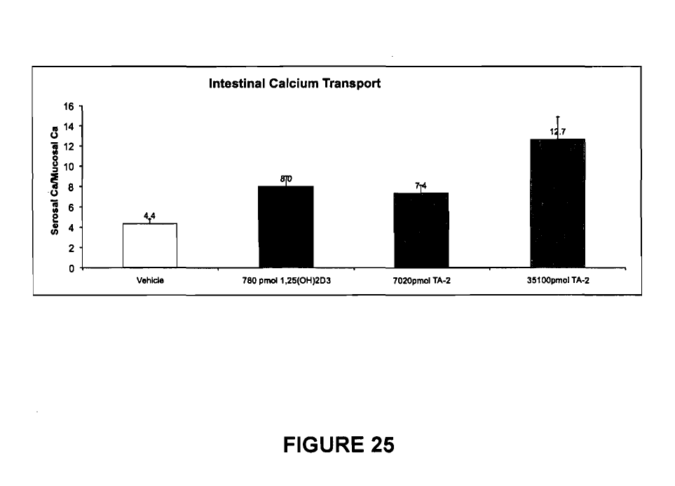

FIG. 25 is a bar graph illustrating the intestinal calcium transport activity of 1,25(OH)₂D₃ as compared to TA-2;

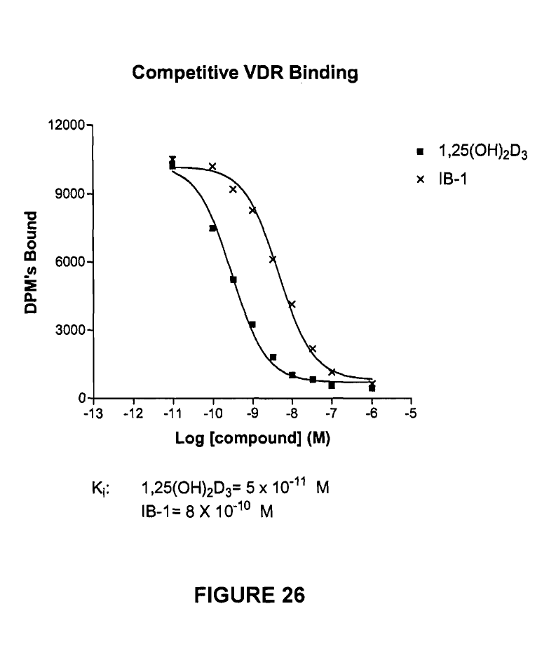

FIG. 26 is a graph illustrating the relative activity of IB-1 and 1,25(OH)₂D₃ to compete for binding with [³H]-1,25-(OH)₂-D₃ to the full-length recombinant rat-vitamin D receptor;

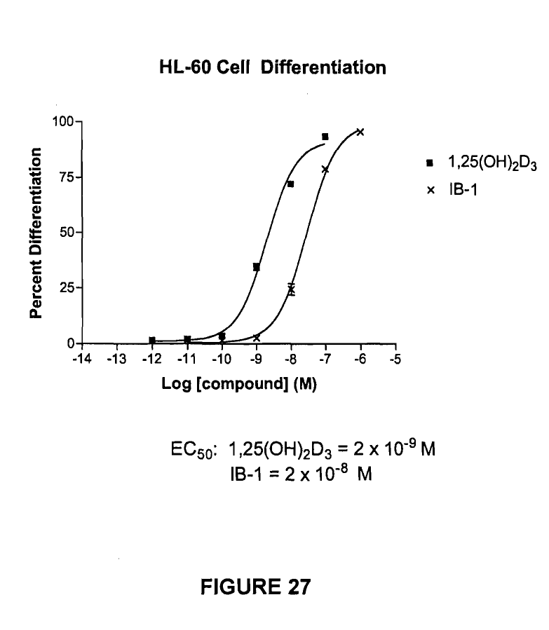

FIG. 27 is a graph illustrating the percent HL-60 cell differentiation as a function of the concentration of IB-1 and 1,25(OH)₂D₃;

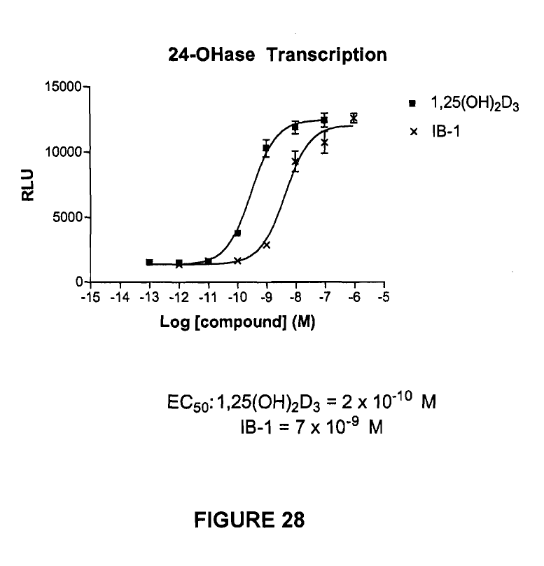

FIG. 28 is a bar graph illustrating the in vitro transcription activity of 1,25(OH)₂D₃ as compared to IB-1;

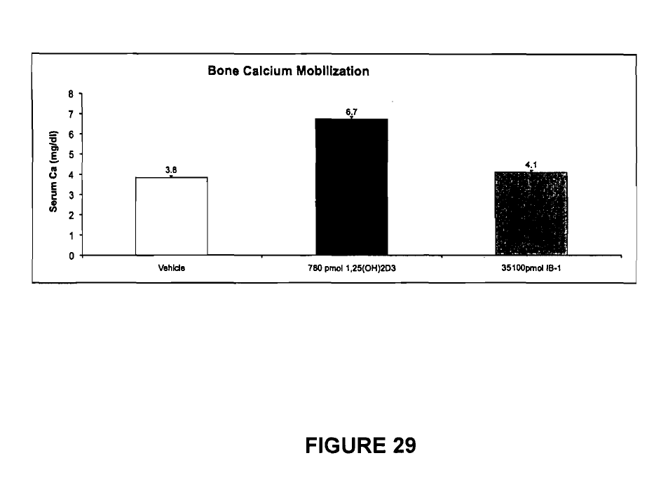

FIG. 29 is a bar graph illustrating the change is serum calcium from baseline of 1,25(OH)₂D₃ as compared to IB-1;

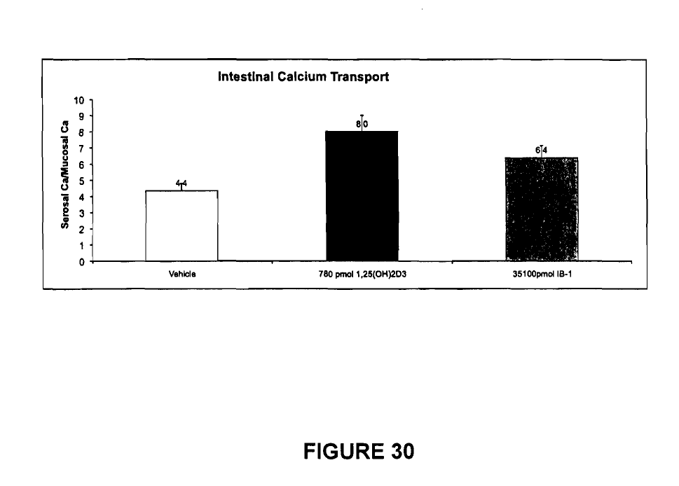

FIG. 30 is a bar graph illustrating the intestinal calcium transport activity of 1,25(OH)₂D₃ as compared to IB-1;

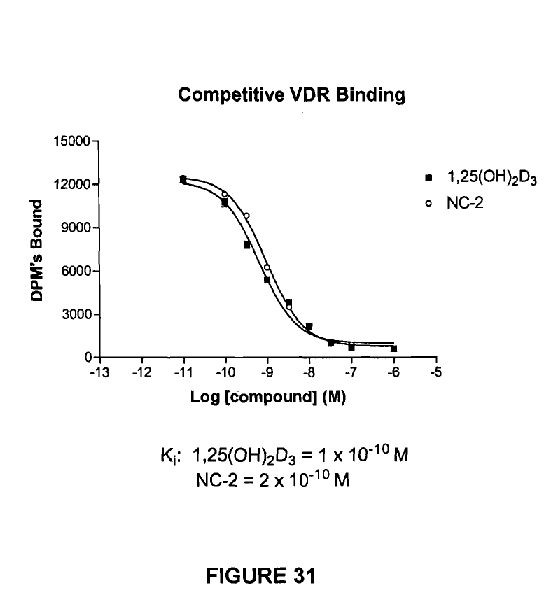

FIG. 31 is a graph illustrating the relative activity of NC-2 and 1,25(OH)₂D₃ to compete for binding with [³H]-1,25-(OH)₂-D₃ to the full-length recombinant rat vitamin D receptor;

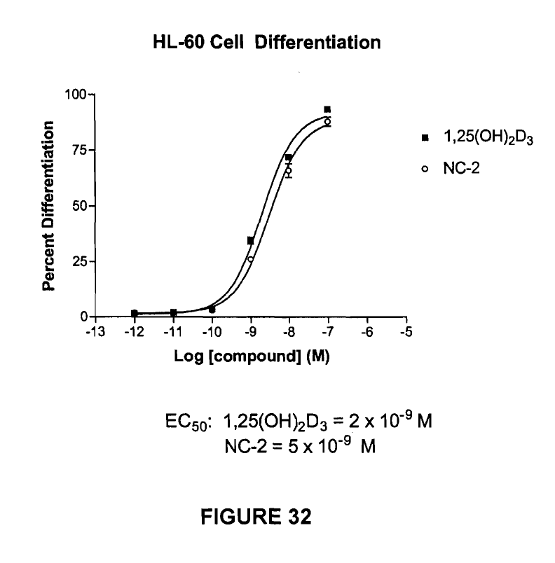

FIG. 32 is a graph illustrating the percent HL-60 cell differentiation as a function of the concentration of NC-2 and 1,25(OH)₂D₃;

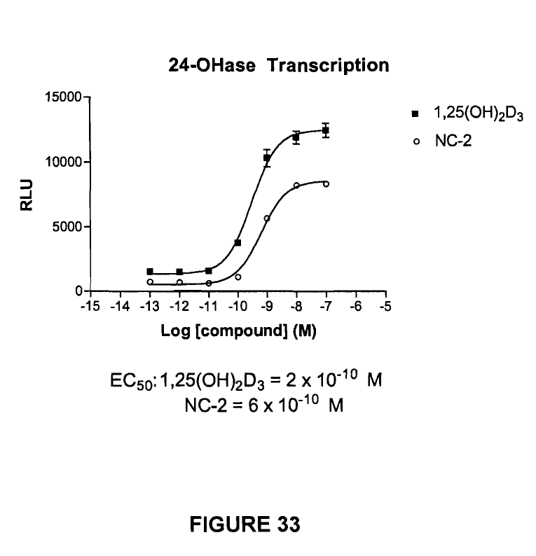

FIG. 33 is a bar graph illustrating the in vitro transcription activity of 1,25(OH)₂D₃ as compared to NC-2;

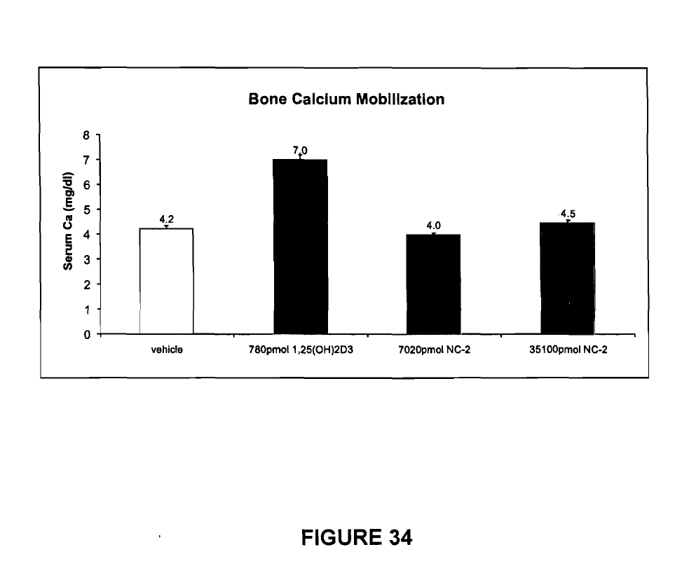

FIG. 34 is a bar graph illustrating the bone calcium mobilization activity of 1,25(OH)₂D₃ as compared to NC-2;

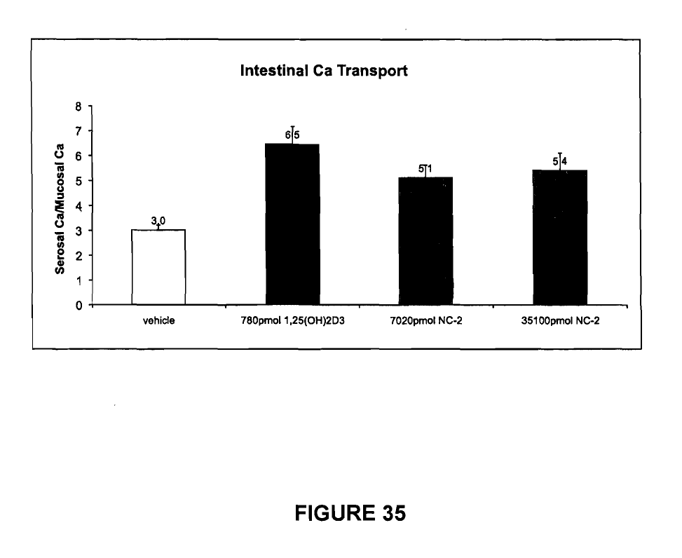

FIG. 35 is a bar graph illustrating, the intestinal calcium transport activity of 1,25(OH)₂D₃ as compared to NC-2;

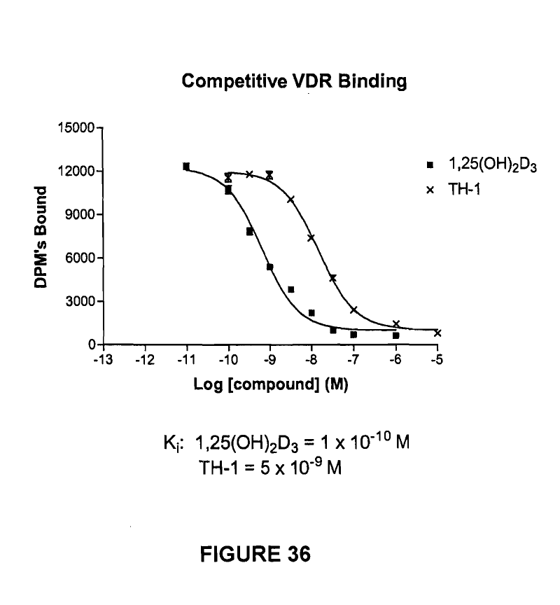

FIG. 36 is a graph illustrating the relative activity of TH-1 and 1,25(OH)₂D₃ to compete, for binding with [³H]-1,25-(OH)₂-D₃ to the full-length recombinant rat vitamin D receptor;

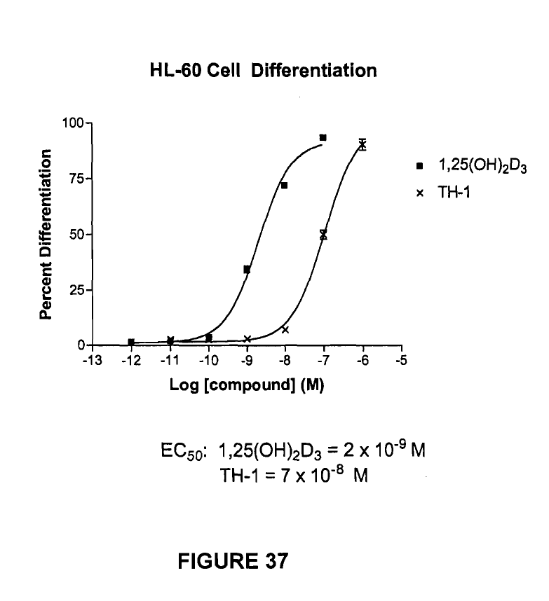

FIG. 37 is a graph illustrating the percent HL-60 cell differentiation as a function of the concentration of TH-1 and 1,25(OH)₂D₃;

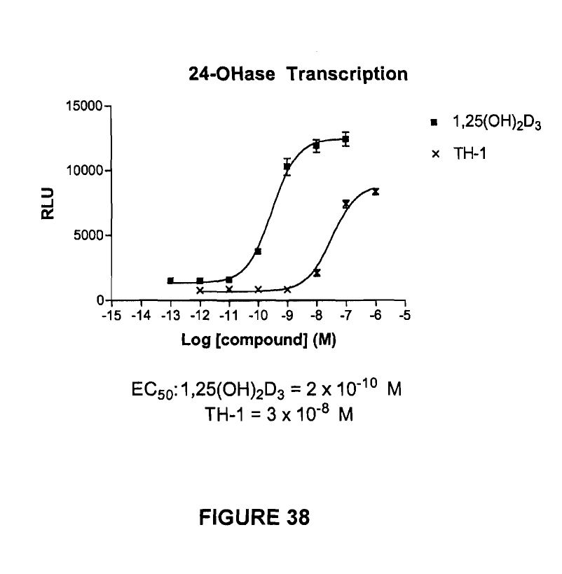

FIG. 38 is a bar graph illustrating the in vitro transcription activity of 1,25(OH)₂D₃ as compared to TH-1;

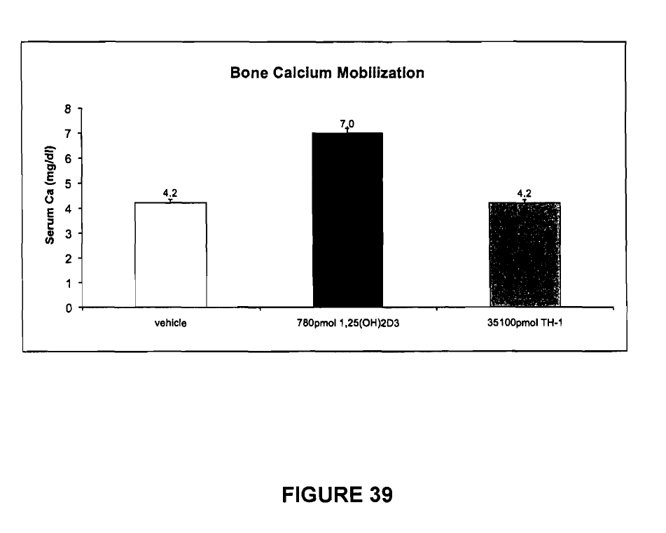

FIG. 39 is a bar graph illustrating the bone calcium mobilization activity of 1,25(OH)₂D₃ as compared to TH-1; and

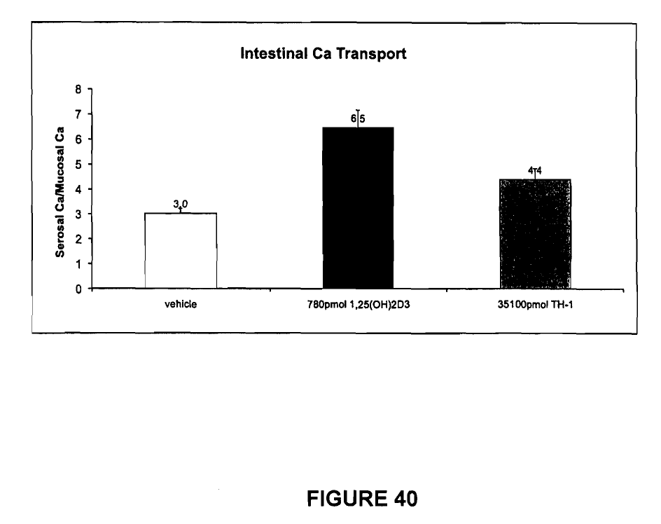

FIG. 40 is a bar graph illustrating the intestinal calcium transport activity of 1,25(OH)₂D₃ as compared to TH-1.

### DETAILED DESCRIPTION OF THE INVENTION

The 2α-methyl and 2β-methyl analogs of 19,26-dinor-1α,25-dihydroxyvitamin D₃ were synthesized and tested. Structurally, these 2α-methyl and 2β-methyl 19-nor analogs are characterized by the general formula I, Ia and Ib respectively previously illustrated herein.

An example of just one 19-nor vitamin D analog that may be administered to a subject or used to prepare a medicament in accordance with the methods of the invention is a compound of formula Va. The compound has the name (20S,25R)-2α-methyl-19,26-dinor-1α,25-dihydroxyvitamin D₃ (LR-2).

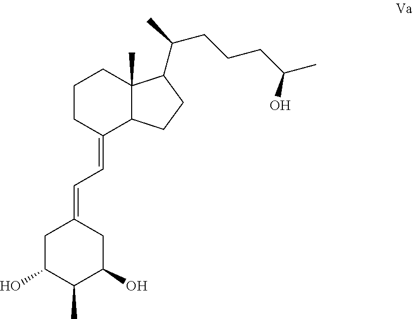

An example of another 19-nor vitamin D analog that may be administered to a subject or used to prepare a medicament in accordance with the methods of the invention is a compound of formula Vb. The compound has the name (20S,25R)-2β-methyl-19,26-dinor-1α,25-dihydroxyvitamin D₃ (FD-1).

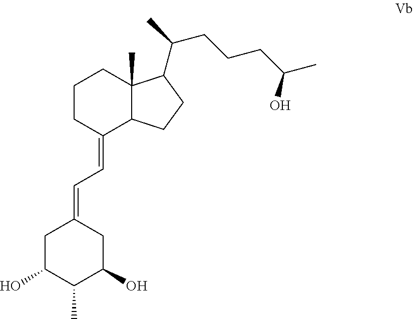

An example of another 19-nor vitamin D analog that may be administered to a subject or used to prepare a medicament in accordance with the methods of the invention is a compound of formula VIa. The compound has the name (20R,25R)-2α-methyl-19,26-dinor-1α,25-dihydroxyvitamin D₃ (MY-2).

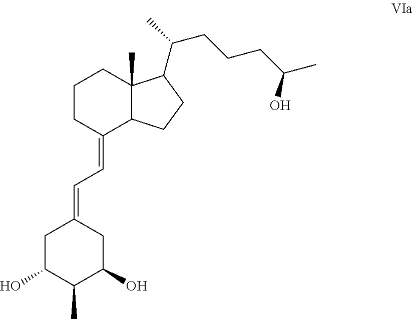

An example of yet another 19-nor vitamin D analog that may be administered to the subject or used to prepare a medicament in accordance with the methods of the invention is a compound of formula VIb. The compound has the name (20R,25R)-2β-methyl-19,26-dinor-1α,25-dihydroxyvitamin D₃ (DW-1).

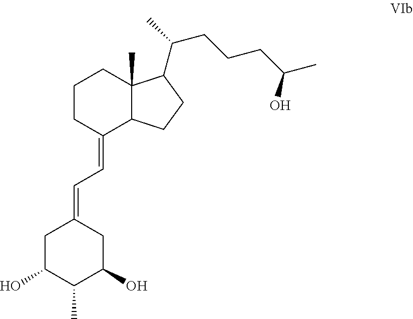

An example of yet another 19-nor vitamin D analog that may be administered to the subject or used to prepare a medicament in accordance with the methods of the invention, is a compound of formula VIIa. The compound has the name (20S,25S)-2α-methyl-19,26-dinor-1α,25-dihydroxyvitamin D₃ (TA-2).

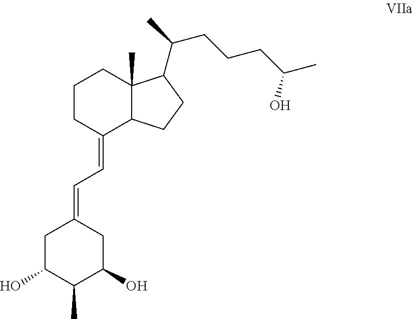

An example of yet another 19-nor vitamin D analog that may be administered to the subject or used to prepare a medicament in accordance with the methods of the invention is a compound of formula VIIb. The compound has the name (20S,25S)-2β-methyl-19,26-dinor-1α,25-dihydroxyvitamin D₃ (IB-1).

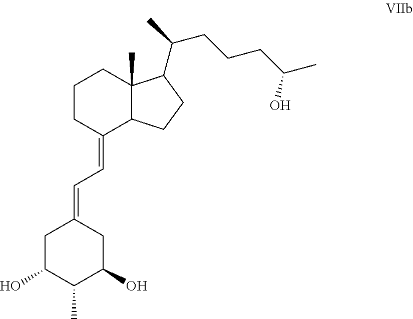

An example of yet another 19-nor vitamin D analog that may be administered to the subject or used to prepare a medicament in accordance with the methods of the invention is a compound of formula VIIIa. The compound has the name (20R,25S)-2α-methyl-19,26-dinor-1α,25-dihydroxyvitamin D₃ (NC-2).

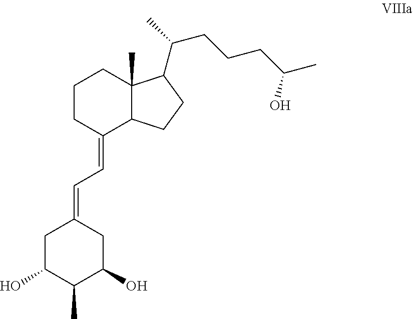

An example of yet another 19-nor vitamin D analog that may be administered to the subject or used to prepare a medicament in accordance with the methods of the invention is a compound of formula VIIIb. The compound has the name (20R,25S)-2β-methyl-19,26-dinor-1α,25-dihydroxyvitamin D₃ (TH-1).

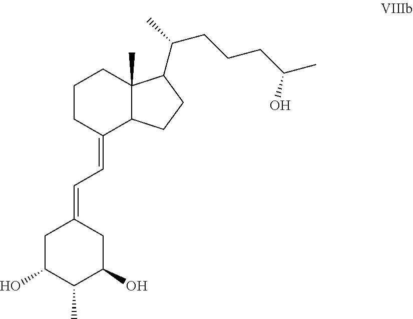

### Synthesis of the Compounds

The preparation of the 2α-methyl and 2β-methyl analogs of 19,26-dinor-1α,25-dihydroxyvitamin D₃ having the basic structure I, Ia and Ib can be accomplished by a common general method, i.e. the condensation of a bicyclic Windaus-Grundmann type ketone II with the allylic phosphine oxide III to the corresponding 2-methylene-19,26-dinor-vitamin D analog IV followed by deprotection at C-1 and C-3 in the latter compound, and finally conversion of the 2-methylene group in IV to a mixture of the 2α-methyl and 2β-methyl compounds of structures Ia and Ib which can then be readily separated to provide both epimers.

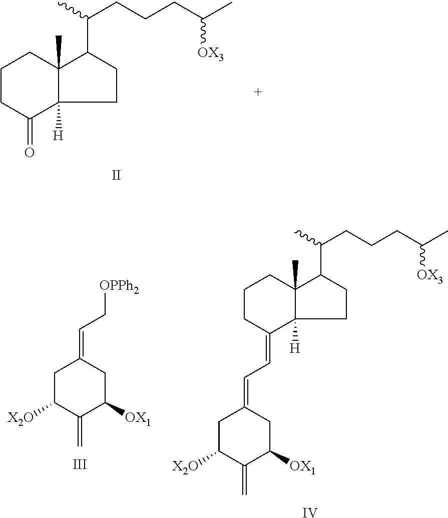

In the structures III and IV, groups X₁ and X₂ and X₃ are hydroxy-protecting groups, preferably t-butyldimethylsilyl. The process shown above represents an application of the convergent synthesis concept, which has been applied effectively for the preparation of vitamin D compounds [e.g. Lythgoe et al., J. Chem. Soc. Perkin Trans. 1, 590 (1978); Lythgoe, Chem. Soc. Rev. 9, 449 (1983); Toh et al., J. Org. Chem. 48, 1414 (1983); Baggiolini et al., J. Org. Chem. 51, 3098 (1986); Sardina et al., J. Org. Chem. 51, 1264 (11986); J. Org. Chem. 51, 1269 (1986); DeLuca et al., U.S. Pat. No. 5,086,191; DeLuca et al., U.S. Pat. No. 5,536,713].

The hydrindanone of the general structure II is not known. It can be prepared by the method shown in the following Schemes (see the preparation of compounds in the illustrative Examples hereinafter described).

For the preparation of the required phosphine oxides of general structure III, a synthetic route has been developed starting from a methyl quinicate derivative which is easily obtained from commercial (1R,3R,4S,5R)-(−)-quinic acid as described by Sicinski et al., J. Med. Chem. 41, 4662 (1998), and by DeLuca and Sicinski, U.S. Pat. No. 5,843,928.

The overall process of the synthesis of a compound of formula I, Ia or Ib is illustrated and described more completely in U.S. Pat. No. 5,945,410 entitled “2-Alkyl-19-Nor-Vitamin D Compounds” the specification of which is specifically incorporated herein by reference.

As used in the description and in the claims, the term “hydroxy-protecting group” signifies any group commonly used for the temporary protection, of hydroxy functions, such as for example, alkoxycarbonyl, acyl, alkylsilyl or alkylarylsilyl groups (hereinafter referred to simply as “silyl” groups), and alkoxyalkyl groups. Alkoxycarbonyl protecting groups are alkyl-O—CO— groupings such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. The term “acyl” signifies an alkanoyl group of 1 to 6 carbons, in all of its isomeric forms, or a carboxyalkanoyl group of 1 to 6 carbons, such as an oxalyl, malonyl, succinyl, glutaryl group, or an aromatic acyl group such as benzoyl, or a halo, nitro or alkyl substituted benzoyl group. The word “alkyl” as used in the description or the claims, denotes a straight-chain or branched alkyl radical of 1 to 10 carbons, in all its isomeric forms. “Alkoxy” refers to any alkyl radical which is attached by oxygen, i.e. a group represented by “alkyl-O—.” Alkoxyalkyl protecting groups are groupings such as methoxymethyl, ethoxymethyl, methoxyethoxymethyl, or tetrahydrofuranyl and tetrahydropyranyl. Preferred silyl-protecting groups are trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals. The term “aryl” specifies a phenyl-, or an alkyl, nitro- or halo-substituted phenyl group.

A “protected hydroxy” group is a hydroxy group derivatised or protected by any of the above groups commonly used for the temporary or permanent protection of hydroxy functions, e.g. the silyl, alkoxyalkyl, acyl or alkoxycarbonyl groups, as previously defined. The terms “hydroxyalkyl”, “deuteroalkyl” and “fluoroalkyl” refer to an alkyl radical substituted by one or more hydroxy, deuterium or fluoro groups respectively. An “alkylidene” refers to a radical having the general formula CₖH₂ₖ— where k is an integer.

More specifically, reference should be made to the following Examples and description as well as to the Schemes herein for a detailed illustration of the preparation of compounds of formula I, and specifically compounds Va, Vb, VIa, VIb, VIIa, VIb, VIIIa and VIIIb.

### Example 1

### Preparation of (20S,25R)-2α-methyl-19,26-dinor-1α,25-dihydroxyvitamin D₃ (LR-2) and (20S,25R)-2β-methyl-19,26-dinor-1α,25-dihydroxyvitamin D₃ (FD-1). See Schemes 1-3

### Preparation of (3R)-1-p-Toluenesulfonyloxy-3-triethylsilyloxy-butane (2)

To a stirred solution of the (R)-(−)-1,3-butanediol 1 (1 g, 11.1 mmol), DMAP (30 mg, 0.25 mmol) and Et₃N (4.6 mL, 3.33 g, 33 mmol) in anhydrous methylene chloride (20 mL) p-toluenesulfonyl chloride (2.54 g, 13.3 mmol) was added at 0° C. The reaction mixture was stirred at 4° C. for 22 h. Methylene chloride was added and the mixture was washed with water, dried (Na₂SO₄) and concentrated under reduced pressure. A residue was chromatographed on silica gel with hexane/ethyl acetate (8:2, then 1:1) to afford the tosylate (2.17 g, 80% yield) as a colorless oil.

To a stirred solution of the tosylate (2.17 g, 8.9 mmol) and 2,6-lutidine (1.14 mL, 1.05 g, 9.8 mmol) in anhydrous methylene chloride (15 mL) triethylsilyl trifluoromethanesulfonate (2 mL, 2.35 g, 8.9 mmol) was added at −50° C. The reaction mixture was allowed to warm to room temperature (4 h) and stirring was continued for additional 20 h. Methylene chloride was added and the mixture was washed with water, dried (Na₂SO₄) and concentrated under reduced pressure. A residue was chromatographed on silica gel with hexane/ethyl acetate (97:3) to afford the product 2 (3.16 g, 99% yield) as a colorless oil:

[α]_{D}−20.7 (c 1.62, CHCl₃); ¹H NMR (400 MHz, CDCl₃) δ 7.77 (2H, d, J=8.2 Hz, o-H_{Ts}), 7.33 (2H, d, J=8.2 Hz, m-H_{Ts}), 4.10 (2H, t, J=6.1 Hz, 1-H₂), 3.90 (1H, m, 3-H), 2.43 (3H, s, Me_{Ts}) 1.72 (2H, m, 2-H₂), 1.10 (3H, d, J=6.2 Hz, 4-H₃), 0.88 (9H, t, J=7.9 Hz 3×SiCH₂CH₃), 0.50 (6H, q, J=7.9 Hz, 3×SiCH₂CH₃); ¹³C NMR (100 MHz) δ 144.62 (s, p-C_{Ts}), 133.02 (s, i-C_{Ts}), 129.72 (d, m-C_{Ts}), 127.82 (d, o-C_{Ts}), 67.78 (t, C-1) 64.45 (d, C-3), 38.46 (t, C-2), 23.81 (q, C-4), 21.51 (q, Me_{Ts}), 6.71 (q, SiCH₂CH₃), 4.76 (t, SiCH₂CH₃); MS (EI) m/z 359 (0.5, MH⁺), 329 (59, M⁺-C₂H₅), 285 (24), 258 (71), 229 (22), 212 (14), 199 (12), 159 (28), 145 (45), 115 (72), 91 (100); exact mass calculated for C₁₅H₂₅O₄SSi (M⁺-C₂H₅) 329.1243, found 329.1248.

### Preparation of (3R)-1-iodo-3-triethylsilyloxy-butane (3)

To a stirred solution of the tosylate 2 (3.15 g, 8.8 mmol) in anhydrous acetone (50 mL) potassium iodide (8 g, 48 mmol) was added and the reaction mixture was refluxed for 10 h. Water (30 mL) was added and the solution was extracted with ethyl acetate. The combined organic phases were dried (Na₂SO₄) and concentrated under reduced pressure. The residue was chromatographed on silica gel with hexane/ethyl acetate a (97:3) to give the alcohol 3 (2.6 g, 94% yield) as a colorless oil:

[α]_{D}−39.5 (c 1.75, CHCl₃); ¹H NMR (400 MHz, CDCl₃) δ 3.89 (1H, m, 3-H), 3.22 (2H, t, J=7.0 Hz, 1-H₂), 1.91 (2H, m, 2-H₂), 1.16 (3H, d, J=6.1 Hz, 4-H₃), 0.96 (9H, t, J=7.9 Hz, 3×SiCH2CH3), 0.61 (6H, q, J=7.9 Hz, 3×SiCH₂CH₃); ¹³C NMR (100 MHz) δ 68.14 (d, C-3), 43.24 (t, C-2), 23.46 (q, C-4), 6.87 (q, SiCH₂CH₃), 5.00 (t, SiCH₂CH₃), 3.37 (t, C-1); MS (EI) m/z 314 (1, M⁺), 299 (3, M⁺-CH₃), 285 (100, M⁺-C₂H₅), 257 (78, M⁺-C₄H₉), 228 (56), 212 (99), 184 (65), 157 (70), 129 (46), 115, (46); exact mass calculated for C₈H₁₈OISi (M⁺-C₂H₅) 285.0172, found 285.0167.

### Preparation of (3R)-Hydroxybutyl-triphenylphosphonium iodide (4)

To a stirred-solution of the iodide 3 (1.24 g, 3.9 mmol) in acetonitrile (50 mL) triphenylphosphine (3.1 g, 11.8 mmol) was added and the reaction mixture was refluxed for 2 days. Acetonitrile was evaporated under reduced pressure, ethyl acetate (59 mL) was added and the mixture was stirred at room temperature for 4 h. After removal of the solvent by filtration the solid was washed with ethyl acetate, filtered off and dried. The pure phosphonium salt 4 (1.74 g, 96% yield) was obtained as white crystals:

¹H NMR (400 MHz CD₃OD) δ 8.00-7.70 (15H, m, H_{Ph}), 3.89 (H, m, 3-H), 3.48 (2H, m, 1-H₂), 1.73 (2H, m, 2-H₂), 1.19 (3H, d, J=6.2 Hz, 4-H₃); ¹³C NMR (100 MHz) δ 136.41 (d, p-C_{Ph}), 134.99 (d, J_{C-P}=10.1 Hz, m-C_{Ph}), 131.70 (d, J_{C-P}=12.1 Hz, o-C_{Ph}), 120.03 (s, J_{C-P}=86.5 Hz, i-C_{Ph}), 67.94 (d, J_{C-P}=17.1 Hz, C-3), 32.52 (t, J_{C-P}=4.0 Hz, C-2), 23.38 (q, C-4), 19.85 (t, J_{C-P}=54.3 Hz, C-1); exact mass calculated for C₂₂H₂₄OPI (M⁺) 335.1565, found 335.1562.

### Preparation of (8S,20S)-des-A,B-20-(hydroxymethyl)pregnan-8-ol (5)

Ozone was passed through a solution of vitamin D₂ (3 g, 7.6 mmol) in methanol (250 mL) and pyridine (2.44 g, 2.5 mL, 31 mmol) for 50 min at −78° C. The reaction mixture was then flushed with an oxygen for 15 min to remove the residual ozone and the solution was treated with NaBH₄ (0.75 g, 20 mmol). After 20 mm the second portion of NaBH₄ (0.75 g, 20 mmol) was added and the mixture was allowed to warm to room temperature. The third portion of NaBH₄ (0.75 g, 20 mmol) was then added and the reaction mixture was stirred for 18 h. The reaction was quenched with water (40 mL) and the solution was concentrated under reduced pressure. The residue was extracted with ethyl acetate and the combined organic phases were washed with 1M aq. HCl, saturated aq. NaHCO₃, dried (Na₂SO₄) and concentrated under reduced pressure. The residue was chromatographed on silica gel with hexane/ethyl acetate (75:25) to give the diol 5 (1.21 g, 75% yield) as white crystals:

m.p. 106-108° C.; [α]_{D}+30.20 (c 1.46, CHCl₃); ¹H NMR (400 MHz, CDCl₃) δ 54.08 (1H, d, J=2.0 Hz, 8α-H), 3.63 (1H, dd, J=10.5, 3.1 Hz, 22-H), 3.38 (1H, dd, J=10.5, 6.8 Hz; 22-H), 1.99 (1H, br.d, J=13.2 Hz), 1.03 (3H, d, J=6.6 Hz, 21-H₃), 0.956, (3H, s, 18-H₃); ¹³C NMR (100 MHz) δ 69.16 (d, C-8), 67.74 (t, C-22), 52.90 (d), 52.33 (d), 41.83 (s, C-13), 40.19 (t), 38.20 (d), 33.53 (t), 26.62 (t), 22.54 (t), 17.36 (t), 16.59 (q, C-21), 13.54 (q, C-18); MS (EI) m/z 212 (2, M⁺), 194 (34, M⁺-H₂O), 179 (33, M⁺-H₂O—CH₃), 163 (18, M⁺-CH₂ OH—H₂O), 135 (36), 125 (54), 111 (100), 95 (63), 81 (67); exact mass calculated for C₁₃H₂₂O (M⁺-H₂O) 194.1671, found 194.1665.

### Preparation of (8S,20S)-des-A,B-8-benzoyloxy-20-(hydroxymethyl)pregnane (6)

Benzoyl chloride (2.4 g; 2 mL, 17 mmol) was added to a solution of the diol 5 (1.2 g, 5.7 mmol) and DMAP (30 mg, 0.2 mmol) in anhydrous pyridine (20 mL) at 0° C. The reaction mixture was stirred at 4° C. for 24 h, diluted with methylene chloride (100 mL), washed with 5% aq. HCl, water, saturated aq. NaHCO₃, dried (Na₂SO₄) and concentrated under reduced pressure. The residue (3.39 g) was treated with a solution of KOH (1 g, 15.5 mmol) in anhydrous ethanol (30 mL) at room temperature. After stirring of the reaction mixture for 3 h, ice and 5% aq. HCl were added until pH=6. The solution was extracted with ethyl acetate (3×50 mL) and the combined organic phases were washed with saturated aq. NaHCO₃, dried (Na₂SO₄) and concentrated under reduced pressure. The residue was chromatographed on silica gel with hexane/ethyl acetate (75:25) to give the alcohol 6 (1.67 g, 93% yield) as a colorless oil:

[α]_{D}+56.0 (c 0.48, —CHCl₃); ¹H NMR (400 MHz, CDCl₃+TMS) δ 8.08-8.02 (2H, m, o-H_{Bz}), 7.59-7.53 (1H, m, p-H_{Bz}), 7.50-7.40 (2H, m, m-H_{Bz}), 5.42 (1H, d, J=24 Hz, 8α-H), 3.65 (1H, dd, J=10.5, 3.2 Hz, 22-H), 3.39 (1H, dd, J=10.5, 6.8 Hz, 22-H), 1.08 (3H, d, J=5.3 Hz, 21-H₃), 1.07 (3H, s, 18-H₃); ¹³C NMR (125 MHz) δ 166.70 (s, C═O), 132.93 (d, p-C_{Bz}), 130.04 (s, i-C_{Bz}), 129.75 (d, o-C_{Bz}), 128.57 (d, m-C_{Bz}), 72.27 (d, C-8); 67.95 (t, C-22), 52.96 (d), 51.60 (d), 42.15 (s, C-13), 39.98 (t), 38.61 (d), 30.73 (t), 26.81 (t), 22.91 (t), 18.20 (t), 16.87 (q, C-21), 13.81 (q, C-18); MS (EI) m/z 316 (5, M⁺), 301 (3, M⁺-Me), 299 (1, M⁺-OH), 298 (2, M⁺-H₂O), 285 (10, M⁺-CH₂OH), 257 (6), 230 (9), 194 (80), 135 (84), 105 (100); exact mass calculated for C₂₀H₂₈O₃ 316.2038, found 316.2019.

### Preparation of (8S,20S)-des-A,B-8-benzoyloxy-20-formylpregnane (7)

Sulfur trioxide pyridine complex (1.94 g, 12.2 mmol) was added to a solution of the alcohol 6 (640 mg; 2.03 mmol), triethylamine (1.41 mL, 1.02 g, 10.1 mmol) in anhydrous methylene chloride (10 mL) and anhydrous DMSO (2 mL) at 0° C. The reaction mixture was stirred under argon at 0° C. for 1 h and then concentrated. The residue was diluted with ethyl acetate, washed with brine, dried (Na₂SO₄) and concentrated. The residue was purified by column chromatography on silica gel with hexane/ethyl acetate (95:5) to give the aldehyde 7 (529 mg, 83% yield) as an oil:

¹H NMR (400 MHz, CDCl₃+TMS) δ 9.60 (1H, d, J=3.1 Hz, CHO), 8.05 (2H, m, o-H_{Bz}), 7.57 (1H, m, p-H_{Bz}), 7.45 (2H, m, m-H_{Bz}), 5.44 (1H, s, 8α-H), 2.39 (1H, m, 20-H), 2.03 (2H, dm, J=11.5 Hz), 1.15 (3H, d, J=6.9 Hz, 21-H₃), 1.10 (3H, s, 18-H₃); ¹³C NMR (100 MHz) δ 204.78 (d, CHO), 166.70 (s, C═O), 132.78 (d, p-Bz), 130.69 (s, i-Bz), 129.50 (d, o-Bz), 128.38, (d, m-Bz), 71.66 (d, C-8), 51.30 (d), 50.95 (d), 49.20 (d), 42.38 (s, C-13), 39.62 (t), 30.47 (t), 25.99 (t), 22.92 (t), 17.92 (t), 13.90 (q), 13.35 (q); MS (EI) m/z 314 (1, M⁺), 299 (0.5, M⁺-Me), 286 (1, M⁺-CO), 285 (5, M⁺-CHO), 257 (1, M⁺-C₃H₅O), 209 (10, M⁺-PhCO), 192 (38), 134 (60), 105 (100), 77 (50); exact mass calculated T for C₂₀H₂₆O₃ 314.1882, found 314.1887.

### Preparation of (8S,20R)-des-A,B-8-benzoyloxy-20-(hydroxymethyl)pregnane (8)

The aldehyde 7 (364 mg, 1.12 mmol) was dissolved in methylene chloride (15 mL) and a 40% aq. n-Bu₄NOH solution (1.47 mL, 1.45 g, 2.24 mmol) was added. The resulting mixture was stirred under argon at room temperature for 16 h, diluted with methylene chloride (20 mL), washed with water, dried (Na₂SO₄) and concentrated under reduced pressure. A residue was chromatographed on silica gel with hexane/ethyl acetate (95:5) to afford a mixture of aldehyde 7 and its 20-epimer (292 mg, 80% yield) in ca 1:2 ratio (by ¹H NMR).

This mixture of aldehydes (292 mg, 0.9 mmol) was dissolved in THF (5 mL) and NaBH₄ (64 mg, 1.7 mmol) was added, followed by a dropwise addition of ethanol (5 mL). The reaction mixture was stirred at room temperature for 30 min and it was quenched with a saturated aq. NH₄Cl solution. The mixture was extracted with ether (3×20 mL) and the combined organic phase was washed with water, dried (Na₂SO₄) and concentrated under reduced pressure. The residue was chromatographed on silica gel with hexane/ethyl acetate (96:4→80:20) to give the desired, pure (20R)-alcohol 8 (160 mg, 55% yield) as an oil and a mixture of 8 and its 20-epimer 6 (126 mg, 43% yield) in ca 1:3 ratio (by ¹H NMR).

[α]_{D}+50.1 (c 1.09, CHCl₃); ¹H NMR (400 MHz, CDCl₃+TMS) δ 8.05 (2H, m, o-H_{Bz}), 7.55 (1H, m, p-H_{BZ}), 7.44 (2H, m, m-H_{Bz}), 5.41 (1H, s, 8α-H), 3.77 (1H, dd, J=10.4, 3.3 Hz, 22-H), 3.45 (1H, dd, J=10.4, 7.4 Hz, 22-H), 1.067 (3H, s, 18-H₃), 0.973 (3H, d, J=6.6 Hz, 21-H₃); ¹³C NMR (100 MHz) δ 166.36 (s, C═O), 132.61 (d, p-C_{Bz}), 130.63 (s, i-C_{Bz}), 129.39 (d, o-C_{Bz}), 128.23 (d, m-C_{Bz}), 71.97 (d, C-8), 66.42 (t, C-22), 52.65 (d), 51.38 (d), 41.58 (s, C-13), 39.16 (t), 37.45 (d), 30.38 (t), 26.29 (t), 22.35 (t), 17.89 (t), 16.42 (q, C-21), 13.78 (q, C-18); MS (EI) m/z 316 (16, M⁺), 301 (5, M⁺-Me), 299 (2, M⁺-OH), 298 (3, M⁺-H₂O), 285 (9, M⁺-CH₂OH), 257 (5), 242 (11), 230 (8), 194 (60), 147 (71), 105 (100); exact mass calculated for C₂₀H₂₈O₃ 316.2038, found 316.2050.

### Preparation of (8S,20R)-des-A,B-8-benzoyloxy-20-formylpregnane (9)

Sulfur trioxide pyridine complex (258 mg, 1.62 mmol) was added to a solution of the alcohol 8 (85 mg, 0.27 mmol), triethylamine (188 μL, 136 mg, 1.35 mmol) in anhydrous-methylene chloride (5 mL) and anhydrous DMSO (1 mL) at 0° C. The reaction mixture was stirred under argon at 0° C. for 1 h and then concentrated. The residue was diluted with ethyl acetate, washed with brine, dried (Na₂SO₄) and concentrated. The residue was purified by column chromatography on silica gel with hexane/ethyl acetate (95:5) to give the aldehyde 9 (70 mg, 83% yield) as an oil:

[α]_{D}+28.8 (c 0.88, CHCl₃); ¹H NMR (500 MHz, CDCl₃) δ 9.55 (1H, d, J=5.0 Hz, CHO), 8.02 (2H, m, o-H_{Bz}), 7.54 (1H, m, p-H_{Bz}), 7.43 (2H, m, m-H_{Bz}), 5.42 (1H, s, 8α-H), 2.35 (1H, m, 20-H), 2.07 (1H, m, 1.87 (1H, m), 1.05 (3H, s, 18-H₃), 1.04 (3H, d, J=7.8 Hz, 21-H₃); ¹³C NMR (125 MHz) δ 205.51 (d, CHO), 166.34 (s, C═O), 132.76 (d, p-C_{Bz}), 130.62 (s, i-C_{Bz}), 129.47 (d, o-C_{Bz}), 128.35, (d, m-C_{Bz}), 71.52 (d, C-8), 52.08 (d), 51.08 (d), 48.40 (d), 41.55 (s, C-13), 38.54 (t), 30.41 (t), 25.28 (t), 22.08 (t), 17.68 (t), 14.49 (q), 13.38 (q); MS (EI) m/z 314 (2, M⁺), 285 (3, M⁺-CHO), 209 (8, M⁺-PhCO), 192 (30, M⁺-PhCOOH), 177 (14), 134 (45), 105 (100), 77 (50); exact mass calculated for C₁₉H₂₅O₂ (M⁺-CHO) 285.1855, found 285.1849.

### Preparation of (8S,20S)-des-A,B-8-benzoyloxy-20-[(4R)-hydroxy-pent-(1E)-enyl]pregnane (10)

To a stirred suspension of the phosphonium salt-4 (221 mg, 0.66 mmol) in anhydrous THF (5 mL) butyllithium (1.6 M, 720 μL, 1.15 mmol) was added at −20° C. The solution turned deep orange. After 1 h a precooled (−20° C.) solution of the aldehyde 9 (70 mg, 0.22 mmol) in anhydrous THF (2 mL) was added and the reaction mixture was, stirred at −20° C. for 3 h hand at room temperature for 18 h. The reaction was quenched with water and the mixture was extracted with ethyl acetate. Combined organic phases were washed with brine, dried (Na₂SO₄) and evaporated. The residue was chromatographed on silica gel with hexane/ethyl acetate (95:5) to give the product 10 (39 mg, 48% yield):

[α]_{D}−28.8 (c 0.8, CHCl₃); ¹H NMR (400 MHz, CDCl₃) δ 8.05 (2H, m, o-H_{Bz}), 7.55 (1H, m, p-H_{Bz}), 7.44 (2H, m, m-H_{Bz}), 5.41 (1H, s, 8α-H), 5.50-5.30 (2H, m, 22-H and 23-H), 3.84 (1H, m, 25-H), 1.20 (3H, d, J=6.2 Hz, 27-H₃), 1.04 (3H, s, 18-H₃), 0.93 (3H, d, J=6.6 Hz, 21-H₃); ¹³C NMR (100 MHz) δ 166.45 (s, C═O), 140.74 (d, C-22), 132.67 (d, p-C_{Bz}), 130.86 (s, i-C_{Bz}), 129.53 (d, o-C_{Bz}), 128.32 (d, m-C_{Bz}), 123.33 (d, C-23), 72.08 (d, C-8), 67.70 (d, C-25), 56.33 (d), 51.48 (d), 42.46 (t), 41.94 (s, C-13), 40.16 (d), 39.48 (t) 30.60 (t), 26.86 (t), 22.74 (q, C-27), 22.50 (t), 21.46 (q, C-21), 17.81 (t), 13.89 (q, C-18); MS (EI) m/z 370 (8, M⁺), 355 (1, M⁺-CH₃), 326 (2, M⁺-C₂H₄O), 284 (12, M⁺-C₅H₁₀O), 265 (2, M⁺-PhCO), 248 (28, M⁺-PhCOOH), 230 (9), 204 (14), 189 (10), 162 (63), 135 (71), 105 (100), exact mass calculated for C₂₄H₃₄O₃Na (MNa⁺) 393.2406, found 393.2407.

### Preparation of (8S,20S-des-A,B-8-benzoyloxy-20-[(4R)-hydroxy-pentyl]pregnane (11)

A solution of the compound 10 (39 mg, 0.11 mmol) in methanol (6 mL) was hydrogenated for 17 h in the presence of 10% palladium on powdered charcoal (6 mg). The reaction mixture was filtered through a bed of Celite with several methanol washes, the filtrate was concentrated and the residue was chromatographed on silica gel with hexane/ethyl acetate (95:5) to give the product 11 (27 mg, 66% yield):

[α]_{D}+18.5 (c 1.0, CHCl₃); ¹H NMR (500 MHz, CDCl₃+TMS) δ 8.05 (2H, m, o-H_{Bz}), 7.56 (1H, m, p-H_{Bz}), 7.44 (2H, m, m-H_{Bz}), 5.41 (1H, d, J=1.8 Hz, 8α-H), 3.80 (1H, m, 25-H), 2.02 (2H, m), 1.81 (2H, m), 1.20 (3H, d, J=6.2 Hz, 27-H₃), 1.04 (3H, s, 18-H), 0.84 (3H, d, J=6.6 Hz, 21-H₃); ¹³C NMR (125 MHz) δ 166.48 (s, C═O), 132.67 (d, p-C_{Bz}), 130.87 (s, i-C_{Bz}), 129.53 (d, o-C_{Bz}), 128.33 (d, m-C_{Bz}), 72.22 (d, C-8), 68.19 (d, C-25), 55.99 (d), 51.63 (d), 41.95 (s, C-13), 39.85 (t), 39.67 (t), 35.19 (t), 34.83 (d), 30.53 (t), 26.94 (t), 23.57 (q, C-27), 22.52 (t), 22.39 (t), 18.47 (q, C-21), 18.06 (t), 13.81 (q, C-18); MS (EI) m/z 372 (12, M⁺), 354 (3, M⁺-H₂O), 339 (0.5, M⁺-H₂O—CH₃), 327 (0.5, M⁺-C₂H₅O), 285 (1, M⁺-C₅H₁₁O), 267 (5, M⁺-PhCO), 250 (60, M⁺-PhCOOH), 232 (24), 163 (24), 135 (63), 105 (100); exact mass calculated for C₂₄H₃₆O₃Na (MNa⁺) 395.2562, found 395.2567.

### Preparation of (8S,20S)-des-A,B-8-benzoyloxy-20-[(4R)-tert-butyldimethylsilyloxy-pentyl]pregnane (12)

tert-Butyldimethylsilyl trifluoromethatiesilfonate (32 μL, 37 mg, 0.14 mmol) was added to a solution of the alcohol 11 (27 mg, 0.07 mmol) and 2,6-lutidine 33 μL, 30 mg, 0.28 mmol) in anhydrous methylene chloride (3 mL) at −20° C. The mixture was stirred under argon at 0° C. for 1 h. The reaction was quenched with water and extracted with methylene chloride. The combined organic phases were washed with brine, dried (Na₂SO₄) and concentrated under reduced pressure. The residue was chromatographed on silica gel with hexane and hexane/ethyl acetate (97:3) to give the product 12 (34 mg, 100%):

[α]_{D}+10.8 (c 1.3, CHCl₃); ¹H NMR (400 MHz, CDCl₃+TMS) δ 8.04 (2H, m, o-H_{Bz}), 7.54 (1H, m, p-H_{Bz}), 7.43 (2H, m, m-H_{Bz}), 5.40 (1H, s, 8α-H), 3.77 (1H, m, 25-H), 2.01 (2H, m), 1.80 (2H, m), 1.11 (3H, d, J=6.0 Hz, 27-H₃), 1.03 (3H, s, 18-H₃), 0.88 (9H, s, Si-t-Bu), 0.82 (3H, d, J=6.5 Hz, 21-H₃), 0.04 (6H, s, SiMe₂); ¹³C NMR (100 MHz) δ 166.50 (s, C═O), 132.66 (d, p-C_{Bz}), 130.91 (s, i-C_{Bz}), 129.55 (d, o-C_{Bz}), 128.33 (d, m-C_{Bz}), 72.26 (d, C-8), 68.72 (d, C-25), 56.02 (d), 51.67 (d), 41.97 (s, C-13), 40.09 (t), 39.85 (t), 35.32 (t), 34.86 (d), 30.58 (t), 26.94 (t), 25.92 (q, SiCM₃), 23.87 (q, C-27), 22.56 (t), 22.36 (t), 18.49 (q, C-21), 18.18 (s, SiCMe₃), 18.07 (t), 13.80 (q, C-18), −4.38 (q, SiMe), −4.68 (q, SiMe), MS (EI) m/z 485 (2, M⁺-H), 471 (2, M⁺-CH₃), 307 (15, M⁺-PhCOOH—C₄H₉), 233 (86, M⁺-PhCOOH-t-BuSiMe₂O), 197 (87), 180 (86), 163 (100), 135 (74), 123 (80), 109 (89); exact mass calculated for C₃₀H₅₀O₃ SiNa (MNa⁺) 509.3427, found 509.3437.

### Preparation of (8S,20S)-des-A,B-20-[(4R)-tert-butyldimethylsilyloxy-pentyl]pregnan-8-ol (13)

A solution of sodium hydroxide in ethanol (2.5M, 2 mL) was added to a stirred solution of the benzoate 12 (34 mg, 70 μmol) in anhydrous ethanol (6 mL) and the reaction mixture was refluxed for 18 h. The mixture was cooled to room temperature, neutralized with 5% aq. HCl and extracted with dichloromethane. Combined organic phases were washed with saturated aq. NaHCO₃, dried (Na₂SO₄) and evaporated. The residue was chromatographed on silica gel with hexane/ethyl acetate (95:5) to give the alcohol 13 (20 mg, 74% yield):

[α]_{D}+8.1 (c 0.75, CHCl₃); ¹H NMR (400 MHz, CDCl₃) δ 4.07 (1H, d, J=1.8 Hz, 8α-H), 3.77 (1H, m, 25-H), 1.96 (1H, m), 1.81 (3H, m), 1.11 (3H, d, J=6.1 Hz, 27-H₃), 0.92 (3H, s, 18-H₃), 0.88 (9H, s, Si-t-Bu), 0.81 (3H, d, J=6.6 Hz, 21-H₃), 0.04 (6H, s, SiMe₂); ¹³C NMR (100 MHz) δ 69.45 (d, C-8), 68.71 (d, C-25), 56.23 (d), 52.65 (d), 41.89 (s, C-13), 40.28 (t), 40.08 (t), 35.26 (t), 34.72 (d), 33.56 (t), 27.04 (t), 25.91 (q, SiCMe₃), 23.84 (q, C-27), 22.42 (t), 22.31 (t), 18.50 (q, C-21), 18.16 (s, SiCMe₃), 17.46 (t), 13.75 (q, C-18)-4.39 (q, SiMe), −4.69 (q, SiMe); MS (EI) m/z 382 (2, M⁺), 367 (6, M⁺-CH₃), 325 (13, M⁺-C₄H₉) 307 (4; M⁺-C₄H₉—H₂O), 233 (57), 191 (44), 177 (50), 163 (60) 151 (53), 135 (55), 123 (58), 93 (65), 75 (100); exact mass calculated for C₁₉H₃₇O₂Si (M⁺-C₄H₉) 325.2563, found 325.2573.

### Preparation of (20S)-des-A,B-20-[(4R)-tert-butyldimethylsilyloxy-pentyl]pregnan-8-one (14)

Molecular sieves A4 (60 mg) were added to a solution of 4-methylmorpholine N-oxide (20 mg, 0.2 mmol) in dichloromethane (0.5 mL). The mixture was stirred at room temperature for 15 min and tetrapropylammonium perruthenate (3 mg, 9 μmol) was added, followed by a solution of alcohol 13 (20 mg, 52 μmol) in dichloromethane (300+300 μL). The resulting suspension was stirred at room temperature for 1 h. The reaction mixture was filtered through a Waters silica Sep-Pak cartridge (5 g) that was 1 h further washed with dichloromethane. After removal of the solvent the ketone 14 (19 mg, 96% yield) was obtained as a colorless oil:

[α]_{D}−33.7 (c 0.7, CHCl₃); ¹H NMR (400 MHz, CDCl₃) δ 3.76 (1H, m, 25-H), 2.44 (1H, dd, J=11.4, 7.7 Hz), 1.12 (3H, d, J=6.0 Hz, 27-H₃), 0.89 (9H, s, Si-t-Bu), 0.84 (3H, d, J=5.9 Hz, 21-H₃), 0.63 (3H, s, 18-H₃), 0.05 (6H, s, SiMe₂); ¹³C NMR (100 MHz) δ 212.13 (s), 68.64 (d, C-25), 62.02 (d), 56.19 (d), 49.94 (s, C-13), 40.96 (t), 40.03 (t), 38.84 (t), 35.52 (t), 34.84 (d), 27.13 (t), 25.89 (q, SiCMe₃), 24.03 (t), 23.84 (q, C-27), 22.27 (t), 18.93 (t), 18.45 (q, C-21), 18.16 (s, SiCMe₃), 12.70 (q, C-18), −4.38 (q, SiMe), −4.70 (q, SiMe); MS (EI) m/z 380 (4, M⁺), 379 (5, M⁺-H), 365 (19, M⁺-CH₃), 351 (7, M⁺-C₂H₅), 323 (100, M⁺-C₄H₈), 231 (87), 189 (64), 175 (60), 161 (75), 149 (70), 135 (97), 121 (65), 95 (81); exact mass calculated for C₂₂H₄₁O₂Si (M⁺-CH₃) 365.2876, found 365.2880.

### Preparation of (20S,25R)-2-Methylene-19,26-dinor-1α,25-dihydroxyvitamin D₃ (17)

To a solution of phosphine oxide 15 (74 mg; 127 μmol) in anhydrous THF (400 μL) at −20° C. was slowly added PhLi (1.8 M in di-n-butylether, 100 μL, 180 μmol) under argon with stirring. The solution turned deep orange. After 30 min the mixture was cooled to −78° C. and a precooled (−78° C.) solution of ketone 14 (19 mg, 50 μmol) in anhydrous THF (200+100 μL) was slowly added. The mixture was stirred under argon at −78° C. for 3 h and at 0° C. for 18 h. Ethyl acetate was added, and the organic phase was washed with brine, dried (Na₂SO₄) and evaporated. The residue was dissolved in hexane and applied on a Waters silica Sep-Pak cartridge (2 g). The cartridge was washed with hexane and hexane/ethyl acetate (99.5:0.5) to give 19-norvitamin derivative 16 (33 mg, 89% yield). Then the Sep-Pak was washed with ethyl acetate to recover diphenylphosphine oxide 15 (48 mg). For analytical purpose a sample of the protected vitamin 16 was further purified by HPLC (9.4×250 mm Zorbax Sil column, 4 mL/min, hexane/2-propanol (99:9:0.1) solvent system, Rₜ=3.45 min):

UV (in hexane) λₘₐₓ 262.6, 253.0, 244.8 nm; ¹H NMR (500 MHz, CDCl₃) δ 6.22 and, 5.84 (each 1H, each d, J=11.2 Hz, 6- and 7-H), 4.97 and 4.92 (each 1H, each s, ═CH₂), 4.43 (2H, m, 1β- and 3α-H), 3.77 (1H, m, 25-H), 2.83 (1H, dm, J=12.6 Hz, 9β-H), 2.51 (1H, dd, J=13.3, 6.0 Hz, 10α-H), 2.46 (H, dd, J=12.6, 4.5 Hz, 4α-H); 2.34 (1H, dd, J=13.3, 2.9 Hz, 10β-H), 2.18 (1H, dd, J=12.6, 8.3 Hz, 4β-H), 1.99 (2H, m), 1.87 (1H, m), 1.12 (3H, d, J=6.1 Hz, 27-H₃), 0.899 (9H, s, Si-t-Bu), 0.892 (9H, s, Si-t-Bu), 0.867 (9H, s, Si-t-Bu), 0.84 (3H, d, J=6.5 Hz, 21-H₃), 0.541 (3H, s, 18-H₃), 0.082 (3H, s, SiMe), 0.068 (3H, s, SiMe), 0.052 (9H, s, 3×SiMe), 0.028 (3H, s, SiMe); ¹³C NMR (125 MHz) δ 152.99 (s, C-2), 141.22 (s, C-8), 132.71 (s, C-5), 122.42 (d, C-6), 116.11 (d, C-7), 106.25 (t, CH₂), 72.52 and 71.65 (each d, C-1 and C-3), 68.74 (d, C-25), 56.32 (d), 56.19 (d), 47.61 (t), 45.70 (s, C-13), 40.50 (t), 40.12 (t), 38.57 (t), 35.62 (t), 35.47 (d), 28.76 (t), 27.37 (t), 25.93 (q, SiCMe₃), 25.84 (q, SiCMe₃), 25.78 (q, SiCMe₃), 23.87 (q, C-27), 23.43 (t), 22.42 (t), 22.11 (t), 18.56 (q, C-21), 18.25 (s, SiCMe₃), 18.17 (s, 2×SiCMe₃), 12.30 (q, C-18), −4.38 (q, SiMe), −4.67 (q, SiMe), −4.87 (q, 3×SiMe), −5.09 (q, SiMe); exact mass calculated for C₄₄H₈₄O₃Si₃Na (MNa⁺) 767.5626, found 767.5612.

The protected vitamin 16 (33 mg, 44 μmol) was dissolved in THF (2 mL) and acetonitrile (2 mL). A solution of aq. 48% HF in acetonitrile (1:9 ratio, 2 mL) was added at 0° C. and the resulting mixture was stirred at room temperature for 6 h. Saturated aq. NaHCO₃ solution was added and the reaction mixture was extracted with ethyl acetate. The combined organic phases were washed with brine, dried (Na₂SO₄) and concentrated under reduced pressure. The residue was diluted with 2 mL of hexane/ethyl acetate (8:2) and applied on a Waters silica Sep-Pak cartridge (2 g). An elution with hexane/ethyl acetate (8:2) and later with ethyl acetate gave the crude product 17 (18 mg). The vitamin 17 was further purified by reverse phase HPLC [9.4×250 mm Zorbax Eclipse XDB-C18 column, 3 mL/min, methanol/water (85:15) solvent system, Rₜ=10.81 min.] to give a colorless oil (13.97 mg, 79% yield):

UV (in EtOH) λₘₐₓ 261.4, 252.4, 244.4 nm; ¹H NMR (500 MHz, CDCl₃) δ 6.35 and 5.88 (1H and 1H, each d, J=11.2 Hz, 6- and 7-H), 5.10 and 5.08 (each 1H, each s, ═CH₂), 4.47 (2H, m, 1β- and 3α-H), 3.78 (1H, m, 25-H), 2.84 (1H, dd, J=13.1, 4.4 Hz, 10β-H), 2.81 (1-H, br d, J=11.9 Hz, 9β-H), 2.56 (1H, dd, J=13.4, 3.6 Hz, 4α-H), 2.32 (1H, dd, J=13.4, 6.1 Hz, 4β-H), 2.28 (1H, dd, J=13.1, 8.4 Hz, 10α-H), 1.18 (3H, d, J=6.2 Hz, 27-H₃), 0.84 (3H, d, J=6.5 Hz, 21-H₃), 0.543 (3H, s, 18-H₃); ¹³C NMR (125 MHz) δ 151.98 (s, C-2), 143.33 (s, C-8), 130.45 (s, C-5), 124.21 (d, C-6), 115.32 (d, C-7), 107.70 (t, ═CH₂), 71.79 and 70.65 (each d, C-1 and C-3); 68.21 (d, C-25), 56.33 (d), 56.14 (d), 45.80 (t), 45.80 (s, C-13), 40.34 (t), 39.69 (t), 38.14 (t), 35.50 (t), 35.39 (d), 28.94 (t), 27.27 (t), 23.53 (q, C-27), 23.48 (t), 22.41 (t), 22.14 (t), 18.52 (q, C-21), 12.34 (q, C-18); MS (EI) m/z 402 (100, M⁺), 384 (9, M⁺-H₂O), 369 (9, M⁺-H₃O—CH₃), 351 (6, M⁺-2H₂O—CH₃), 317 (31), 287 (38 M⁺-C₇H₁₅O), 269 (39), 251 (36), 192 (19), 161 (40), 147 (65), 135 (85); exact mass calculated for C₂₆H₄₂O₃ (M⁺) 402.3134, found 402.3127.

### Preparation of (20S,25R)-2α-methyl-19,26-dinor-10,25-dihydroxyvitamin D₃ (18) and (20S,25R)-2β-methyl-19,26-dinor-1α,25-dihydroxyvitamin D₃ (19)

Tris(triphenylphosphine)rhodium (I) chloride (9 mg, 9.7 μmol) was added to dry benzene (5 mL) presaturated with hydrogen (15 min). The mixture was stirred at room temperature until a homogeneous solution was formed (ca. 25 min). A solution of vitamin 17 (2.6 mg, 6.5 μmmol) in dry benzene (3 mL) was then added and the reaction was allowed to proceed under a continuous stream of hydrogen for 4 h. Benzene was removed under vacuum, the residue was redissolved in hexane/ethyl acetate (1:1) and applied on a Waters silica Sep-Pak cartridge (2 g). A mixture of 2-methyl vitamins was eluted with the same solvent system. The compounds were further purified by HPLC (9.4×250 mm Zorbax Sil column, 4 mL/min) using hexane/2-propanol (85:15) solvent system. The mixture of 2-methyl-19-norvitamins; 18 and 19 gave a single peak at Rₜ=9.1 min. Separation of both epimers was achieved by reversed-phase HPLC (9.4×250 mm Zorbax RX C18 column, 3 mL/min) using methanol/water (85:15) solvent system. 2β-Methyl vitamin 19 (455 μg, 17% yield) was collected at Rₜ=8.5 min. and its 2α-epimer 18 (492 μg, 19% yield) at Rₜ=11.4 min:

2α-Methyl analog 18 UV (in EtOH) λₘₐₓ 260.0, 250.0, 243.5 nm; ¹H NMR (500 MHz, CDCl₃) δ 6.37 and 5.82 (1H and 1H, each d, J=11.3 Hz, 6- and 7-H), 3.95 (1H, m, 1β-H), 3.79 (1H, m, 25-H), 3.61 (1H, m, 3α-H), 2.80 (2H, br m, 9β- and 10α-H), 2.60 (1H, dd, J=12.9, 4.5 Hz, 4α-H), 2.22 (1H, br d, J=12.8 Hz, 10β-H), 2.13 (1H, ˜t, J˜11.2 Hz, 4β-H), 1.98 (2H, m), 1.191 (3H, d, J=6.2 Hz, 27-H₃), 1.134 (3H, d, J=6.8 Hz, 2α-CH₃) 0.846 (3H, d, J=6.5 Hz, 21-H₃), 0.530 (3H, s, 18-H₃); MS (EI) m/z 404 (100, M⁺), 386 (36, M⁺-H₂O), 368 (31, M⁺-2HO₂), 350 (51, M⁺-3H₂O), 317 (17, M⁺-C₅H₁₀OH), 289 (50, M⁺-C₇H₁₄OH), 271 (43, M⁺-C₇H₁₄OH—H₂O), 253 (73), 231 (38), 199 (37), 159 (48) 147 (68), 135 (76); exact mass calculated for C₂₆H₄₄O₃ (M⁺) 404.3290, found 404.3284.

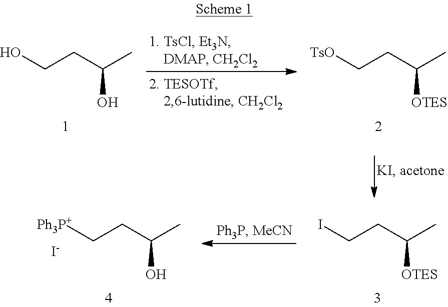

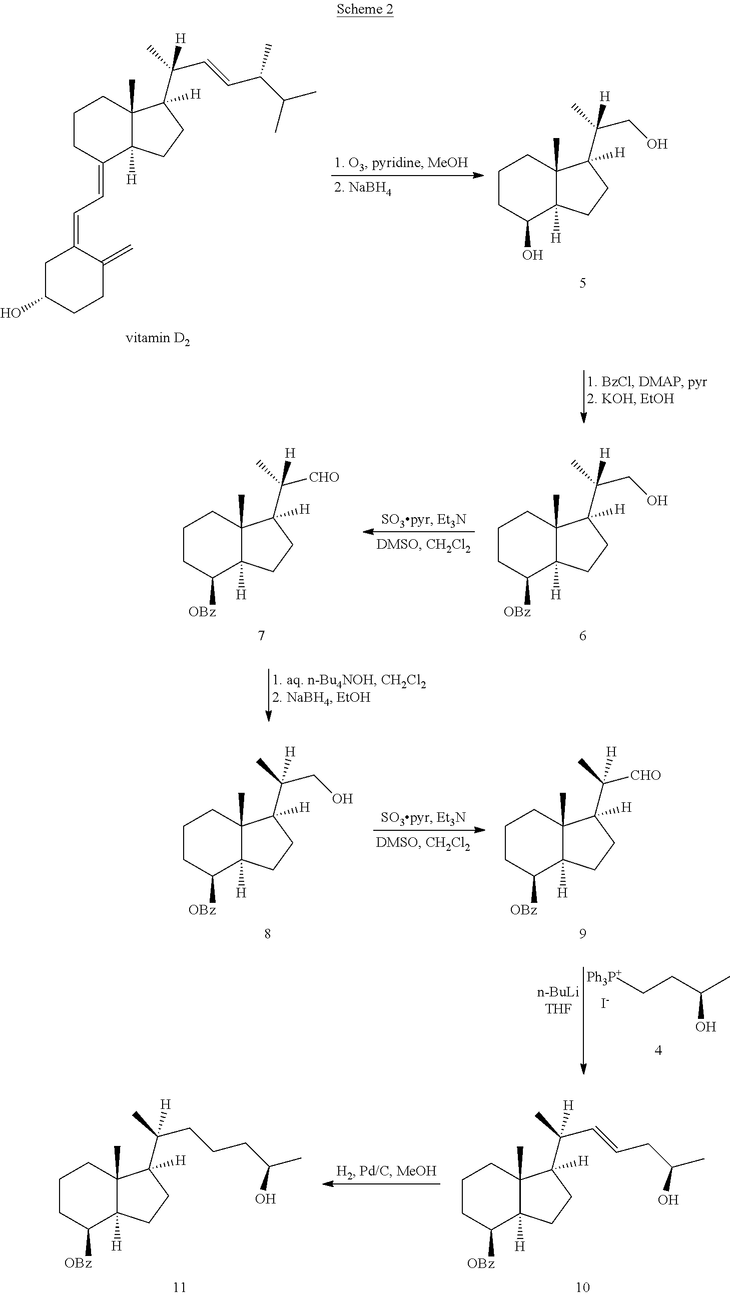

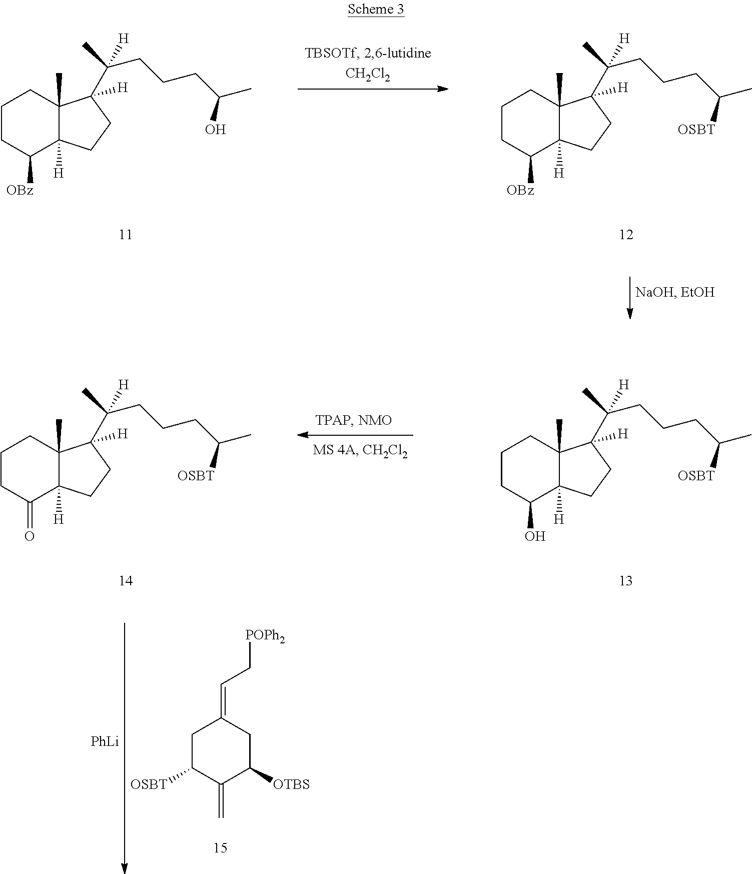

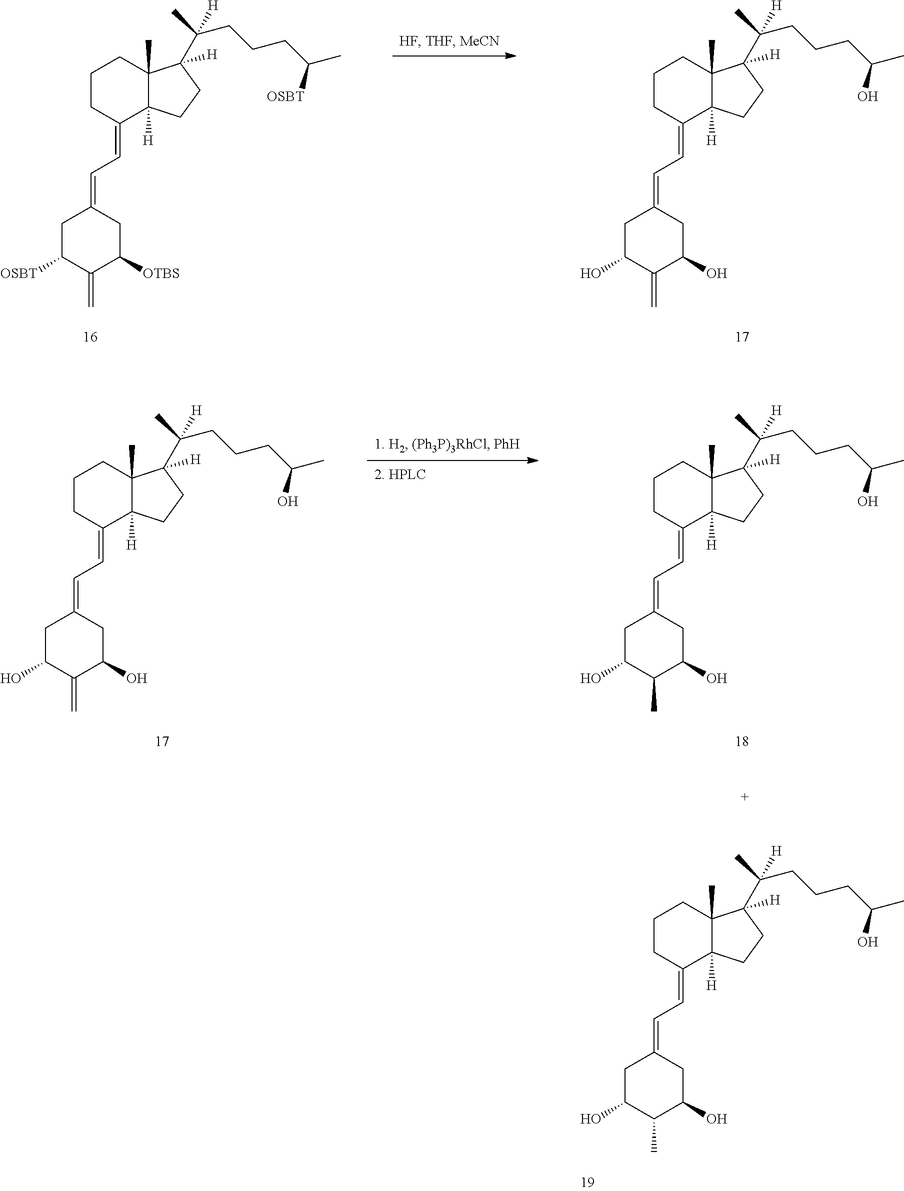

### Example 2

### Preparation of (20R,25R)-2α-methyl-19,26-dinor-1α,25-dihydroxyvitamin D₃ (MY-2) and (20R,25R)-2β-methyl-19,26-dinor-1α,25-dihydroxyvitamin D₃ (DW-1). See Schemes 4-6

### Preparation of (3R)-1-p-Toluenesulfonyloxy-3-triethylsilyloxy-butane (2a)

To a stirred solution of the (R)-(−)-1,3-butanediol 1a (1 g, 11.1 mmol), DMAP (30 mg, 0.25 mmol) and Et₃N (4.6 mL, 3.33 g, 33 mmol) in anhydrous methylene chloride (20 mL) p-toluenesulfonyl chloride (2.54 g, 13.3 mmol) was added at 0° C. The reaction mixture was stirred at 4° C. for 22 h. Methylene chloride was added and the mixture was washed with water, dried (Na₂SO₄) and concentrated under reduced pressure. A residue was chromatographed on silica gel with hexane/ethyl acetate (8:2, then 1:1) to afford the tosylate (2.17 g, 80% yield) as a colorless oil.

To a stirred solution of the tosylate (2.17 g, 8.9 mmol) and 2,6-lutidine (1.14 mL, 1.05 g, 9.8 mmol) in anhydrous methylene chloride (15 mL) triethylsilyl trifluoromethanesulfonate (2 mL, 2.35 g, 8.9 mmol) was added at −50° C. The reaction mixture was allowed to warm to room temperature (4 h) and stirring was continued for additional 20 h. Methylene chloride was added and the mixture was washed with water, dried (Na₂SO₄) and concentrated under reduced pressure. A residue was chromatographed on silica gel with hexane/ethyl acetate (97:3) to afford the product 2a (3.16 g, 99% yield) as a colorless oil:

[α]_{D}−20.7 (c 1.62, CHCl₃), ¹H NMR (400 MHz, CDCl₃) δ 7.77 (2H, d, J=8.2 Hz, o-H_{Ts}), 7.33 (2H, d, J=8.2 Hz, m-H_{Ts}), 4.10 (2H, t, J=6.1 Hz, 1-H₂), 3.90 (1H, m, 3-H), 2.43 (3H, s, Me_{Ts}), 1.72 (2H m, 2-H₂), 1.10 (3H, d, J=6.2 Hz, 4-H₃), 0.88 (9H, t, J=7.9 Hz, 3×SiCH₂CH₃), 0.50 (6H, q, J=7.9 Hz, 3×SiCH₂CH₃); ¹³C NMR (100 MHz) δ 144.62 (s, p-C_{Ts}), 133.02 (s, i-C_{Ts}), 129.72 (d, m-C_{Ts}), 127.82 (d, o-C_{Ts}), 67.78 (t, C-1), 64.45 (d, C-3), 38.46 (t, C-2), 23.81 (q, C-4), 21.51 (q, Me_{Ts}), 6.71 (q, SiCH₂CH₃), 4.76 (t, SiCH₂CH₃); MS (EI) m/z 359 (0.5, MH⁺), 329 (59, M⁺-C₂H₅), 285 (24), 258 (71), 229 (22), 212 (14), 199 (12), 159 (28), 145 (45), 115 (72), 91 (100); exact mass calculated for C₁₅H₂₅O₄SSi (M⁺-C₂H₅) 329.1243, found 329.1248.

### Preparation of (3R)-1-Iodo-3-triethylsilyloxy-butane (3a)

To a stirred solution of the tosylate 2a (3.15 g, 8.8 mmol) in anhydrous acetone (50 mL) potassium iodide (8 g, 48 mmol) was added and the reaction mixture was refluxed for 10 h. Water (30 mL) was added and the solution was extracted with ethyl acetate. The combined organic phases were dried (Na₂SO₄) and concentrated under reduced pressure. The residue was chromatographed on silica gel with hexane/ethyl acetate (97:3) to give the alcohol 3a (2.6 g, 94% yield) as a colorless oil:

[α]_{D}−39 5 (c 1.75, CHCl₃); ¹H NMR (400 MHz, CDCl₃) δ 3.89 (1H, m, 3-H), 3.22 (2H, t, J=7.0 Hz, 1-H₂), 1.91 (2H, m, 2-H₂), 1.16 (3H, d, J=6.1 Hz, 4-H₃), 0.96 (9H, t, J=7.9 Hz, 3×SiCH₂CHₑ), 0.61 (6H, q, J=7.9 Hz, 3×SiCH₂CH₃); ¹³C NMR (100 MHz) δ68.14 (d, C-3), 43.24 (t, C-2), 23.46 (q, C-4), 6.87 (q, SiCH₂CH₃), 5.00 (t, SiCH₂ CH₃), 3.37 (t, C-1); MS (EI) m/z 314 (1, M⁺), 299 (3, M⁺-CH₃), 285 (100, M⁺-C₂H₅), 257 (78, M⁺-C₄H₉), 228 (56), 212 (99), 184 (65), 157 (70); 129 (46), 115 (46); exact mass calculated for C₈H₁₈OISi (M⁺-C₂H₅) 285.0172, found 285.0167.

### Preparation of (3R)-Hydroxybutyl-triphenylphosphonium iodide (4a)

To a stirred solution of the iodide 3a (1.24 g, 3.9 mmol) in acetonitrile (50 mL) triphenylphosphine, (3.1 g, 11.8 mmol) was added and the reaction mixture was refluxed for 2 days. Acetonitrile was evaporated under reduced pressure, ethyl acetate (50 mL) was added and the mixture was stirred at room temperature for 4 h. After removal of the solvent by filtration the solid was washed with ethyl acetate, filtered off and dried. The pure phosphonium salt 4a (1.74 g, 96% yield) was obtained as white crystals:

¹H NMR (400 MHz, CD₃OD) δ 8.00-7.70 (15H, m, H_{Ph}), 3.89 (1H, m, 3-H), 3.48 (2H, m, 1-H₂), 1.73 (2H, m, 2-H₂), 1.19 (3H, d, J=6.2 Hz, 4-H₃); ¹³C NMR (100 MHz) δ 136.41 (d, p-C_{Ph}), 134.99 (d, J_{C-P}=10.1 Hz, m-C_{Ph}), 131.70 (d, J_{C-P}=12.1 Hz, o-C_{Ph}), 120.03 (s, J_{C-P}=86.5 Hz, i-C_{Ph}), 67.94 (d, J_{C-P}=17.1 Hz, C-3), 32.52 (t, J_{C-P}=4.0 Hz, C-2), 23.38 (q, C-4), 19.85 (t, J_{C-P}=54.3 Hz, C-1); exact mass calculated for C₂₂H₂₄OPI (M⁺) 335.1565, found 335.1562.

### Preparation of (8S,20S)-de-A,B-20-(hydroxymethyl)pregnan-8-ol (5a)

Ozone was passed through a solution of vitamin D₂ (3 g, 7.6 mmol) in methanol (250 mL) and pyridine (2.44 g, 2.5 mL, 31 mmol) for 50 min at −78° C. The reaction mixture was then flushed with an oxygen for 15 min to remove the residual ozone and the solution was treated with NaBH₄ (0.75 g, 20 mmol). After 20 ml the second portion of NaBH₄ (0.75 g, 20 mmol) was added and the mixture was allowed to warm to room temperature. The third portion of NaBH₄ (0.75 g, 20 mmol) was then added and the reaction mixture was stirred for 18 h. The reaction was quenched with water (40 mL) and the solution was concentrated under reduced pressure. The residue was extracted with ethyl acetate and the combined organic phases were washed with 1M aq. HCl, saturated aq. NaHCO₃, dried (Na₂SO₄) and concentrated under reduced pressure. The residue was chromatographed on silica gel with hexane/ethyl acetate (75:25) to give the diol 5a (1.21 g, 75% yield) as white crystals:

m.p. 106-108° C.; [α]_{D}+30.20 (c 1.46, CHCl₃); ¹H NMR (400 MHz, CDCl₃) δ 4.08 (1H, d, J=2.0 Hz 8α-H), 3.63 (1H, dd, J=10.5, 3.1 Hz, 22-H), 3.38 (1H, dd, J=10.5, 6.8 Hz 22-H), 1.99 (1H, br.d, J=13.2 Hz), 1.03 (3H, d, J=6.6 Hz, 21-H₃), 0.956 (3H, s, 18-H₃); ¹³C NMR (100 MHz) δ 69.16 (d, C-8), 67.74 (t, C-22), 52.90 (d), 52.33 (d), 41.83 (s, C-13), 40.19 (t), 38.20 (d), 33.53 (t), 26.62 (t), 22.54 (t), 17.36 (t), 16.59 (q, C-21), 13.54 (q, C-18); MS (EI) m/z 212 (2, M⁺), 194 (34, M⁺-H₂O), 179 (33, M⁺-H₂O—CH₃), 163 (18, M⁺-CH₂OH—H₂O), 135 (36), 125 (54), 111 (100), 95 (63), 81 (67); exact mass calculated for C₁₃H₂₂O (M⁺-H₂O) 194.1671, found 194.1665.

### Preparation of (8S,20S)-de-A,B-8-benzoyloxy-20-(hydroxymethyl)pregnane (6a)

Benzoyl chloride (2.4 g, 2 mL, 17 mmol) was added to a solution of the diol 5a (1.2 g, 5.72 mmol) and DMAP (30 mg, 0.2 mmol) in anhydrous pyridine (20 mL) at 0° C. The reaction mixture was stirred at 4° C. for 24 h, diluted with methylene chloride (100 mL), washed with 5% aq. HCl, water, saturated aq. NaHCO₃, dried (Na₂SO₄) and concentrated under reduced pressure. The residue (3.39 g) was treated with a solution of KOH (1 g, 15.5 mmol) in anhydrous ethanol (30 mL) at room temperature. After stirring of the reaction mixture for 3 h, ice and 5% aq. HCl were added until pH=6. The solution was extracted with ethyl acetate (3×50 mL) and the combined organic phases were washed with saturated aq. NaHCO₃, dried (Na₂SO₄) and concentrated under reduced pressure. The residue was chromatographed on silica gel with hexane/ethyl acetate (75:25) to give the alcohol 6a (1.67 g, 93% yield) as a colorless oil:

[α]_{D}+56.0 (c 0.48, CHCl₃); ¹H NMR (400. MHz, CDCl₃+TMS) δ 88-8.02 (2H, m, o-H_{Bz}), 7.59-7.53 (1H, m, p-H_{Bz}), 7.50-7.40 (2H, m, m-H_{Bz}), 5.42 (1H, d, J=2.4 Hz, 8α-H), 3.65 (1H, dd, J=10.5, 3.2 Hz, 22-H), 3.39 (1H, dd, J=10.5, 6.8 Hz, 22-H), 1.08 (3H, d, J=5.3 Hz, 21-H₃), 1.07 (3H, s, 18-H₃); ¹³C NMR (125 MHz) δ 166.70 (s, C═O) 132.93 (d, p-C_{Bz}) 131.04 (s, i-C_{Bz}), 129.75 (d, o-C_{Bz}), 128.57 (d, m-C_{Bz}), 72.27 (d, C-8), 67.95 (t, C-22), 52.96 (d), 51.60 (d), 42.15 (s, C-13), 39.98 (t), 38.61 (d), 30.73 (t), 26.81 (t), 22.91 (t), 18.20 (t), 16.87 (q, C-21), 13.81 (q, C-18); MS (EI) m/z 316 (5, M⁺), 301 (3, M⁺-Me), 299 (1, M⁺-OH), 298 (2, M⁺-H₂O), 285 (10, M⁺-CH₂OH), 257 (6), 230 (9), 194 (80), 135 (84), 105 (100); exact mass calculated for C₂₀H₂₈O₃ 316.2038, found 316.2019.

### Preparation of (8S,20S)-de-A,B-8-benzoyloxy-20-formylpregnane (7a)

Sulfur trioxide pyridine complex (1.94 g, 12.2 mmol) was added to a solution of the alcohol 6a (640 mg, 2.03 mmol), triethylamine (1.41 mL, 1.02 g, 10.1 mmol) in anhydrous methylene chloride (10 mL) and anhydrous DMSO (2 mL) at 0° C. The reaction mixture was stirred under argon at 0° C. for 1 h and then concentrated. The residue was diluted with ethyl acetate, washed with brine, dried (Na₂SO₄) and concentrated. The residue was purified by column chromatography on silica gel with hexane/ethyl acetate (95:5) to give the aldehyde 7a (529 mg, 83% yield) as an oil: [α]_{D}+63.1 (c 5.85, CHCl₃); ¹H NMR (400 MHz, CDCl₃+TMS) δ 9.60 (1H, d, J=3.1 Hz, CHO), 8.05 (2H, m, o-H_{Bz}), 7.57 (1H, m, p-H_{Bz}), 7.45 (2H, m, m-H_{Bz}), 5.44 (1H, s, 8α-H), 2.39 (1H, m, 20-H), 2.03 (2H, dm, J=11.5 Hz), 1.15 (3H, d, J=6.9 Hz, 21-H₃), 1.10 (3H, s, 18-H₃); ¹³C NMR (100 MHz) δ 204.78 (d, CHO), 132.78 (d, p-Bz), 130.69 (s, i-Bz), 129.50 (d, o-Bz), 128.38, (d, m-Bz), 71.66 (d, C-8), 51.30 (d), 50.95 (d), 49.20 (d), 42.38 (s, C-13), 39.62 (t), 30.47 (t), 25.99 (t), 22.92 (t), 17.92 (t), 13.90 (q), 13.35 (q), MS (EI) m/z 314 (1, M⁺), 299 (0.5, M⁺-Me), 286 (1, M⁺-CO), 285 (5 M⁺-CHO), 257 (1, M⁺-C₃H₅O), 209 (10, M⁺-PhCO), 192 (38), 134 (60), 105 (100), 77 (50); exact mass calculated for C₂₀H₂₆O₃ 314.1882, found 314.1887.

### Preparation of (8S,2R)-de-A,B-8-benzoyloxy-20-[(4R)-hydroxy-pent-(1E)-en-yl]pregnane (8a)

To a stirred suspension of the phosphonium salt 4a (361 mg, 0.78 mmol) in anhydrous THF (5 mL) butyllithium (1.6 M, 980 μL, 1.56 mmol) was added at −20° C. The solution turned deep orange. After 1 h a precooled (−20° C.) solution of the aldehyde 7a (81 mg 0.26 mmol) in anhydrous THF (2 mL) was added and the reaction mixture was stirred at −20° C. for 3 h and at room temperature for 18 h. The reaction was quenched with water and the mixture was extracted with ethyl acetate. Combined organic phases were washed with brine, dried (Na₂SO₄) and evaporated. The residue was chromatographed on silica gel with hexane/ethyl acetate (95:5) to give the product 8a (47 mg, 49% yield):

[α]_{D}+69.6 (c 1.3, CHCl₃); ¹H NMR (400 MHz, CDCl₃+TMS) δ 8.05 (2H, m, o-H_{Bz}), 7.56 (1H, m, p-H_{Bz}), 7.45 (2H, m, m-H_{Bz}), 5.41 (1H, s, 8α-H), 5.40-5.20 (2H, m, 22-H and 23-H), 3.78 (1H, m, 25-H), 1.18 (3H, d, J=6.1 Hz, 27-H₃), 1.07 (3H, s, 18-H₃), 1.05 (3H, d, J=6.8 Hz, 21-H₃); ¹³C NMR (100 MHz) δ 166.44 (s, C═O), 140.80 (d, C-22), 132.16 (d, p-C_{Bz}), 130.84 (s, i-C_{Bz}), 129.51 (d, o-C_{Bz}), 128.32 (d, m-C_{Bz}), 123.25 (d, C-23), 72.14 (d, C-8), 67.20 (d, C-25), 55.97 (d), 51.64 (d), 42.37 (t), 41.84 (s, C-13), 39.91 (d), 39.80 (t), 30.49 (t), 27.58 (t), 22.57 (t), 22.57 (q, C-27), 20.59 (q, C-21), 17.99 (t), 13.72 (q, C-18); MS (EI) m/z 370 (12, M⁺), 352 (1, M⁺-H₂O), 326 (4, M⁺-C₂H₄O), 284 (18, M⁺-C₅H₁₀), 248 (40, M⁺-PhCOOH), 230 (12), 204 (31), 189 (16), 162 (97), 134 (81), 121 (61), 106 (63), 93 (66), 77 (100); exact mass calculated for C₂₄H₃₄O₃ (M⁺) 370.2508, found 370.2503.

### Preparation of (8S,20R)-de-A,B-8-benzoyloxy-20-[(4R)-hydroxy-pentyl]pregnane (9a)

A solution of the compound 8a (46 mg, 0.12 mmol) in methanol (6 mL) was hydrogenated for 17 h in the presence of 10% palladium on powdered charcoal (7 mg). The reaction mixture was filtered through a bed of Celite with several methanol washes, the filtrate was concentrated and the residue was chromatographed on silica gel with hexane/ethyl acetate (95:5) to give the product 9a (31 mg, 69% yield):

[α]_{D}+61.3 (c 0.65, CHCl₃); ¹H NMR (400 MHz, CDCl₃+TMS) δ 8.06 (2H, m, o-H_{Bz}), 7.56 (1H, m, p-H_{Bz}), 7.45 (2H, m, m-H_{Bz}), 5.41 (1H, d, J=1.5 Hz, 8α-H), 3.80 (1H, m, 25-H), 2.04 (2H, m), 1.83 (2H, m), 1.19 (3H, d, J=6.2 Hz, 27-H₃), 1.04 (3H, s, 18-H₃), 0.95 (3H, d, J=6.5 Hz, 21-H₃); ¹³C NMR (100 MHz) δ 166.50 (s, C═O), 132.66 (d, p-C_{Bz}), 130.91 (s, i-C_{Bz}), 129.54 (d, o-C_{Bz}), 128.33 (d, m-C_{Bz}), 72.25 (d, C-8), 68.27 (d, C-25), 56.33 (d), 51.61 (d), 41.92 (s, C-13), 39.92 (t), 39.84 (t), 35.70 (t), 35.37 (d), 30.55 (t), 27.09 (t), 23.49 (q, C-27), 22.64 (t), 22.21 (t), 18.55 (q, C-21), 18.02 (t), 13.53 (t, C-18), MS (EI) m/z 372 (11, M⁺), 354 (2, M⁺-H₂O), 327 (0.5, M⁺-C₂H₅O), 285 (1, M⁺-C₅H₁₁O), 267 (4, M⁺-PhCO), 250 (58, M⁺-PhCOOH), 232 (28), 217 (7), 163 (31), 135 (67), 105 (100); exact mass calculated for C₂₄H₃₆O₃ (M⁺) 372.2664, found 372.2672.

### Preparation of (8S,20R)-de-A,B-8-benzoyloxy-20-[(4R)-tert-butyldimethylsilyloxy-pentyl]pregnane (10a)

tert-Butyldimethylsilyl trifluoromethanesulfonate (37 μL, 42 mg, 0.16 mmol) was added to a solution of the alcohol 9a (30 mg, 0.08 mmol) and 2,6-lutidine (37 μL, 34 mg, 0.32 mmol) in anhydrous methylene chloride (3 mL) at −20° C. The mixture was stirred under argon at 0° C. for 1 h. The reaction was quenched with water and extracted with ethylene chloride. The combined organic phases were washed with brine, dried (Na₂SO₄) and concentrated under reduced pressure. The residue was chromatographed on silica gel with hexane and hexane/ethyl acetate (97:3) to give the product 10a (39 mg, 100%):

[α]_{D}+2.7 (c 0.85, CHCl₃); ¹H NMR (400 MHz, CDCl₃) δ 8.06 (2H, m, o-H_{Bz}), 7.55 (1H, m, p-H_{Bz}), 7.44 (2H, m, m-H_{Bz}), 5.41 (1H, s, 8α-H), 3.77 (1H, m, 25-H), 2.04 (2H, m), 1.84 (2H, m), 1.11 (3H, d, J=6.0 Hz, 27-H₃), 1.04 (3H, s, 18-H₃), 0.93 (3H, d, J=6.5 Hz, 21-H₃), 0.89 (9H, s, Si-t-Bu), 0.05 (6H, s, SiMe₂); ¹³C NMR (100 MHz) δ 166.50 (s, C═O), 132.65 (d, p-C_{Bz}), 130.93 (s, i-C_{Bz}), 129.55 (d, o-C_{Bz}), 128.33 (d, m-C_{Bz}), 72.27 (d, C-8), 68.68 (d, C-25), 56.51 (d), 51.63 (d), 41.92 (s, C-13), 40.20 (t), 39.96 (t), 35.74 (t), 35.40 (d), 30.57 (t), 27.09 (t), 25.91 (q, SiCMe₃), 23.81 (q, C-27), 22.65 (t), 22.25 (t), 18.51 (q, C-21), 18.17 (s, SiCMe₃), 18.04 (t), 13.54 (q, C-18), −4.37 (q, SiMe), −4.68 (q, SiMe); MS (EI) m/z 485 (1, M⁺-H), 471 (1, M⁺-CH₃), 307 (16, M⁺-PhCOOH—C₄H₉), 233 (40, M⁺-PhCOOH-t-BuSiMe₂O), 197 (58), 179 (55), 159 (79), 137 (64), 123 (80), 109 (100); exact mass calculated for C₂₆H₄₁O₃Si (M⁺-C₄H₉) 429.2825, found 429.2843.

### Preparation of (8S,20R)-de-A,B-20-[(4R)-tert-butyldimethylsilyloxy-pentyl]pregnan-ol (11a)

A solution of sodium hydroxide in ethanol (2.5 M, 2 mL was added to a stirred solution of the benzoate 10a (38 mg, 78 μmol) in anhydrous ethanol (10 mL) and the reaction mixture was refluxed for 18 h. The mixture was cooled to room temperature, neutralized with 5% aq. HCl and extracted with dichloromethane. Combined organic phases were washed with saturated aq. NaHCO₃, dried (Na₂SO₄) and evaporated. The residue was chromatographed on silica gel with hexane/ethyl acetate (95:5) to give the alcohol 11a (22 mg, 74% yield):

[α]_{D}+19.2 (c 0.4, CHCl₃); ¹H NMR (400 MHz, CDCl₃+TMS) δ 4.07 (1H, d, J=1.6 Hz 8α-H), 3.77 (1H, m, 25-H), 2.00 (1H, m), 1.82 (3H, m), 1.11 (3H, d, J=6.1 Hz, 27-H₃), 0.93 (3H, s, 18-H₃), 0.89 (3H, d, 21-H₃) covered by 0.89 (9H, s, Si-t-Bu), 0.05 (6H, s, SiMe₂); ¹³C NMR (100 MHz) δ 69.46 (d, C-8), 68.72 (d, C-25), 56.76 (d), 52.65 (d), 41.87 (s, C-13), 40.43 (t), 40.25 (t), 35.78 (t), 35.24 (d), 33.61 (t), 27.15 (t), 25.92 (q, SiCMe₃), 23.81 (q, C-27), 22.53 (t), 22.30 (t), 18.47 (q, C-21), 18.16 (s, SiCMe₃), 17.45 (t), 13.53 (q, C-18), −4.37 (q, SiMe), −4.68 (q, SiMe); MS (EI) m/z 382 (0.5, M⁺), 367 (1, M⁺-CH₃) 325 (3, M⁺-C₄H₉), 307 (3, M⁺-C₄H₉—H₂O), 233 (48), 191 (22), 177 (38), 163 (60), 135 (79), 123 (61), 109 (76), 97(84), 75 (100); exact mass calculated for C₁₉H₃₇O₂Si (M⁺-C₄H₉) 325.2563, found 325.2574.

### Preparation of (20R)-de-A,B-20-[(4R)-tert-butyldimethylsilyloxy-pentyl]pregnan-8-one, (12a)

Pyridinium dichromate (110 mg, 293 μmol) was added to a solution of the alcohol 11a (22 mg, 58 μmol) and pyridinium p-toluenesulfonate (3 mg, 12 μmol) in anhydrous methylene chloride (6 mL). The resulting suspension was stirred at room temperature for 3 h. The reaction mixture was filtered through a Waters silica Sep-Pak cartridge (5 g) that was further washed with hexane/ethyl acetate (8:2). After removal of solvents the ketone 12a (18 mg, 82% yield) was obtained as a colorless oil: [α]_{D}−4.8 (c 1.05, CHCl₃); ¹H NMR (400 MHz, CDCl₃+TMS) δ 3.77 (1H, m, 25-H), 2.44 (1H, dd, J=11.5, 7.5 Hz), 1.12 (3H, d, J=6.1 Hz, 27-H₃), 0.95 (3H, d, J=6.0 Hz, 21-H₃), 0.89 (9H, s, Si-t-Bu), 0.64 (3H, s, 18-H₃), 0.05 (6H, s, SiMe₂); ¹³C NMR (100 MHz) δ 211.99 (s, C═O), 68.63 (d, C-25), 62.01 (d), 56.78 (d), 49.92 (s, C-13), 40.96 (t), 40.15 (t), 39.03 (t), 35.79 (t), 35.47 (d), 27.50 (t), 25.90 (q, SiCMe₃), 24.05 (t), 23.79 (q, 27), 22.24 (t), 19.06 (t), 18.64 (q, C-21), 18.15 (s, SiCMe₃), 12.47 (q, C-18), −4.36 (q, SiMe), −4.70 (q, SiMe); MS (EI) m/z 379 (3, M⁺-H), 365 (11, M⁺-CH₃), 323 (75, M⁺-C₄H₉), 231 (46), 189 (55), 175 (78), 161 (100), 149 (90); exact mass calculated for C₁₉H₃₅O₂Si (M⁺-C₄H₉) 323.2406, found 323.2420.

### Preparation of (20R,25R)-2-Methylene-19,26-dinor-1α,25-dihydroxyvitamin D₃ (15a)

To a solution of phosphine oxide 13a (105 mg, 180 μmol) in anhydrous THF (1 mL) at −20° C. was slowly added PhLi (1.8 M in di-n-butylether, 120 μL, 216 μmol) under argon with stirring. The solution turned deep orange. After 30 min the mixture was cooled to −78° C. and a precooled (−78° C.) solution of ketone 12a (18 mg, 47 μmol) in anhydrous THF (300+200 μL) was slowly added. The mixture was stirred under argon at −78° C. for 3 h and at 0° C. for 18 h. Ethyl acetate was added, and the organic phase was washed with brine, dried (Na₂SO₄) and evaporated. The residue was dissolved in hexane and applied on a Waters silica Sep-Pak cartridge (2 g). The cartridge was washed with hexane and hexane/ethyl acetate (99:5:0.5) to give 19-norvitamin derivative 14a (35.5 mg, 100% yield); then the Sep-Pak was washed with ethyl acetate to recover diphenylphosphine oxide 13a (62 mg):

UV (in hexane) λₘₐₓ 263.2, 253.2, 244.6 nm; ¹H NMR (400MHz, CDCl₃) δ 6.22 and 5.85 (each 1H, each d, J=11.1 Hz, 6- and 7-H), 4.98 and 4.93 (each 1H, each s, ═CH₂), 4.43 (2H, nm, 1β- and 3α-H), 3.78 (1H, m, 25-H), 2.83 (1H, dm, J=12.1 Hz, 9β-H), 2.52 (1H, dd, J=13.3, 6.1 Hz, 10α-H), 2.47 (1H, dd, J=12.9, 4.4 Hz, 4α-H), 2.34 (1H, dd, J=13.3, 2.8 Hz, 10β-H), 2.18 (1H, dd, J=12.5, 8.6 Hz, 4β-H), 2.00 (2H, m), 1.12 (3H, d, J=6.0 Hz, 27-H₃), 0.93 (3H, d, J=6.4 Hz, 21-H₃), 0.901 (9H, s, Si-t-Bu), 0.897 (9H, s, Si-t-Bu), 0.871 (9H, s, Si-t-Bu), 0.551 (3H, s, 18-H₃), 0.084 (3H, s, SiMe), 0.071 (3H, s, SiMe), 0.056 (9H, s, 3×SiMe), 0.031 (3H, s, SiMe); ¹³C NMR (100 MHz) δ 153.03 (s, C-2), 141.24 (s, C-8), 132.70 (s, C-5), 122.45 (d, C-6), 116.13 (d, C-7), 106.24 (t, ═CH₂), 72.55 and 71.69 (each d, C-1 and C-3), 68.73 (d, C-25), 56.68 (d), 56.33 (d), 47.64 (t), 45.70 (s, C-1.3), 40.66 (t), 40.24 (t), 38.61 (t), 36.11 (d), 35.94 (t), 28.78 (t), 27.72 (t), 25.94 (q, SiCMe₃), 25.85 (q, SicMe₃), 25.80 (q, SiCMe₃), 23.80 (q, C-27), 23.47 (t), 22.39 (t), 22.24 (t), 18.77 (q, C-21), 18.26 (s, SiCMe₃), 18.17 (s, 2×SiCMe₃), 12.09 (q, C-18), −4.35 (q, SiMe), −4.66 (q, SiMe), −4.85 (q, 2×SiMe), −4.88 (q, SiMe), −5.07 (q, SiMe); MS (EI) m/z 497 (24, M⁺-t-BuMe₂SiOH-t-BuMe₂Si), 480 (11, M⁺-2 t-BuMe₂SiOH), 366 (61), 351 (24), 271 (15), 257 (24), 234 (33), 197 (25), 147 (36) 73 (100); exact mass calculated for C₄₄H₈₄O₃Si₃Na (MNa⁺) 767.5626, found 767.5640.

The protected vitamin 14a (35.4 mg, 48 μmol) was dissolved in THF (4 mL) and acetonitrile (4 mL). A solution of aq. 48% HF in acetonitrile (1.9 ratio, 4 mL) was added, at 0° C. and the resulting mixture was stirred at room temperature for 2 h. Saturated aq. NaHCO₃ solution was added and the reaction mixture was extracted with ethyl acetate. The combined organic phases were washed with brine, dried (Na₂SO₄) and concentrated under reduced pressure. The residue was diluted with 2 mL of hexane/ethyl, acetate (9:1) and applied on a Waters silica Sep-Pak cartridge (2 g). An elution with hexane/ethyl acetate (9:1, then 7:3) gave the crude product 15a (21 mg). The vitamin 15a was further purified by reverse phase HPLC [9.4×250 mm Zorbax Eclipse XDB-C18 column, 4 mL/min, methanol/water (85:15) solvent system, Rₜ=9.7 min.] to give a colorless oil (15.06 mg, 78% yield):

UV (in EtOH) λₘₐₓ 262.0, 252.5, 244.3 nm; ¹H NMR (600 MHz, CDCl₃) δ 6.35 and 5.88 (1H and 1H, each d, J=11.2 Hz, 6- and 7-H), 5.11 and 5.01 (each 1H, each s, ═CH₂), 4.47 (2H, 1β- and 3α-H), 3.80 (1H, m, 25-H), 2.84 (1H, dd, J=13.3, 4.5 Hz, 10-H), 2.81 (1H, m, 9-H), 2.57 (1H, dd, J=13.3, 3.7 Hz, 4α-H), 2.32 (1H, dd, J=13.3, 6.2 Hz, 4β-H) 2.29 (1H, dd, J=13.3, 8.4 Hz, 10α-H), 1.19 (3H, d, J=6.2 Hz, 27-H₃), 0.93 (3H; d, J=6.3 Hz, 21-H₃), 0.551 (3H, s, 18-H₃); ¹³C NMR (100 MHz) δ 152.02 (s, C-2), 143.36 (C-8), 130.44 (s, C-5), 124.22 (d, C-6), 115.31 (d, C-7), 107.67 (t, ═CH₂), 71.80 and 70.68 (each d, C-1 and C-3), 68.29 (d, C-25), 56.49 (d), 56.33 (d), 45.80 (t), 45.80 (s, C-13), 40.47 (t), 39.87 (t), 38.17 (t), 36.05 (d), 35.90 (t), 28.96 (t), 27.64 (t), 23.49 (q, C-27), 23.49 (t, 22.29 (2×t), 18.78 (q, C-21), 12.08 (q, C-18); MS (EI) m/z 402 (58, M⁺), 384 (4, M⁺-H₂O), 369 (4, M⁺-H₂O—CH₃), 351 (3, M⁺-2H₂O—CH₃), 317 (18), 287 (21, M⁺-C₇H₁₅O), 269 (21), 251 (21), 233 (38), 177 (33), 163 (54), 135 (92), 105 (100); exact mass calculated for C₂₆H₄₂O₃(M⁺) 40.3134, found 402.3142.

### Preparation of (20R,25R)-2α-dimethyl-19,26-dinor-1α,25-dihydroxyvitamin D₃ (16a) and (20R,25R)-2β-methyl-19,26-dinor-1α,25-dihydroxyvitamin D₃ (17a)

Tris(triphenylphosphine)rhodium (I) chloride (7 mg, 7.6 μmol) was added to dry benzene (5 mL) presaturated with hydrogen (15 min). The mixture was stirred at room temperature until a homogeneous solution was formed (ca. 25 min). A solution of vitamin 15a (3.09 mg, 7.7 μmol) in dry benzene (3 mL) was then added and the reaction was allowed to proceed under a continuous stream of hydrogen for 4 h. Benzene was removed under vacuum, the residue was redissolved in hexane/ethyl acetate (1:1) and applied on a Waters silica Sep-Pak cartridge (2 g). A mixture of 2-methyl vitamins was eluted with the same solvent system. The compounds were further purified by HPLC (9.4×250 mm Zorbax-Sil column, 4 mL/min) using hexane/2-propanol (85:15) solvent system. The mixture of 2-methyl-19-norvitamins 16a and 17a gave a single peak at Rₜ=9.4 min. Separation of both epimers was achieved by reversed-phase HPLC (9.4×250 mm Zorbax RX C18 column, 3 mL/min) using methanol/water (85:15) solvent system. 2β-Methyl vitamin 17a (1.227 mg, 39% yield) was collected at Rₜ=9.4 min. and its 2α-epimer 16a (1.32 mg, 42% yield) at Rₜ=10.1 min:

2α-Methyl analog 16a: UV (in EtOH) λₘₐₓ 260.0, 251.0, 243.5 nm; ¹H NMR (500 MHz, CDCl₃) δ 6.37 and 5.82 (1H and 1H, each d, J=11.2 Hz, 6- and 7-H), 3.96 (1H, m, 1β-H), 3.79 (1H, m, 25-H), 3.61 (1H, m, 3α-H), 2.80 (2H, br m, 9β- and 10α-H), 2.60 (1H, dd, J=13.0, 4.4 Hz, 4α-H), 2.22 (1H, br d, J=13.4 Hz, 10β-H), 2.13 (1H, ˜t, J˜11.3 Hz, 4β-H), 1.191 (3H, d, J=6.2 Hz, 27-H₃), 1.131 (3H, d, J=6.8 Hz, 2α-CH₃), 0.927 (3H, d, J=6.5 Hz, 21-H₃), 0.531 (3H, s, 18-H₃); MS (EI) m/z 404 (100, M⁺), 386 (23, M⁺-H₂O), 368 (14, M⁺-2H₂O), 350 (23, M⁺-3H₂O), 335 (6, M⁺-3H₂O—CH₃), 317 (16, M⁺-C₅H₁₀OH), 289 (50, M⁺-C₇H₁₄OH), 271 (33, M⁺-C₇H₁₄OH—H₂O), 253 (43), 231 (18), 194 (23), 161 (32), 147 (46), 135 (54); exact mass calculated for C₂₆H₄₄O₃ (M⁺) 404.3290, found 404.3281.

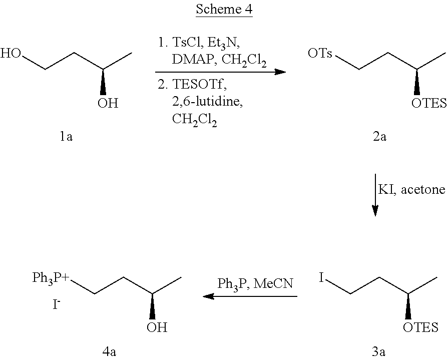

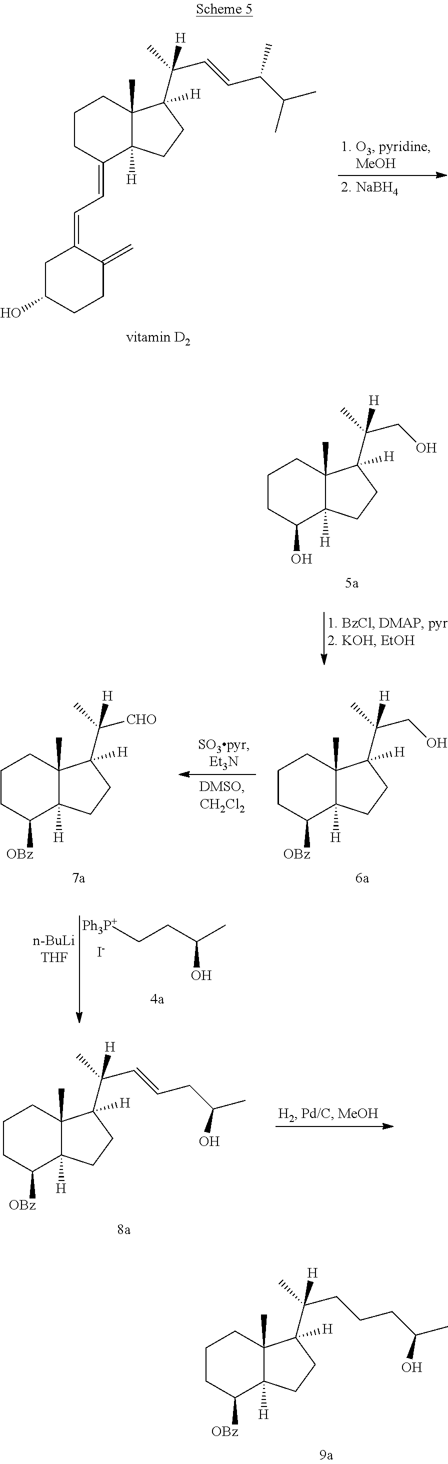

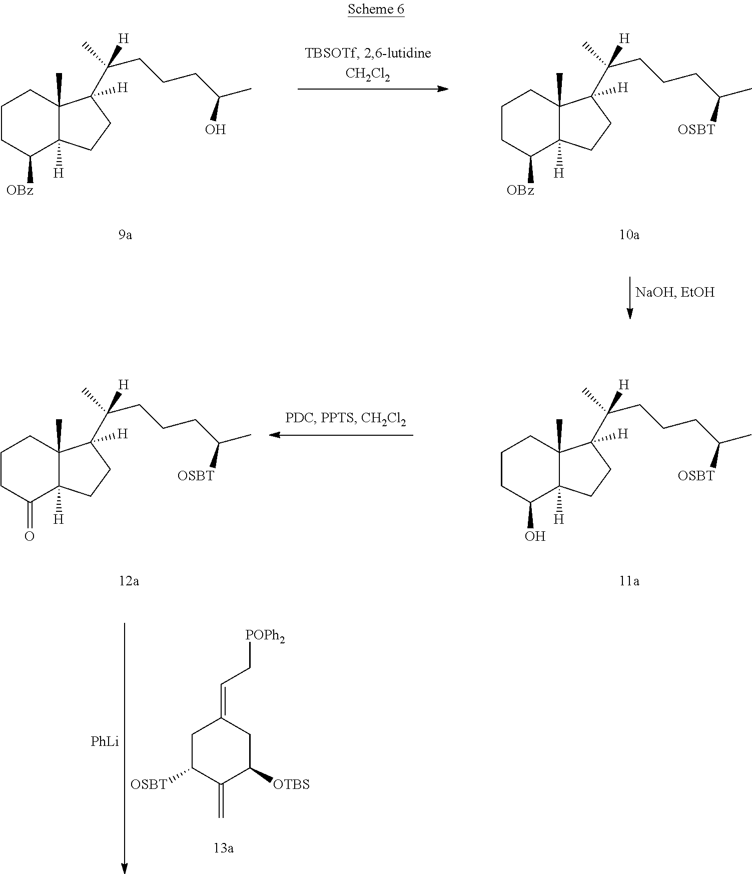

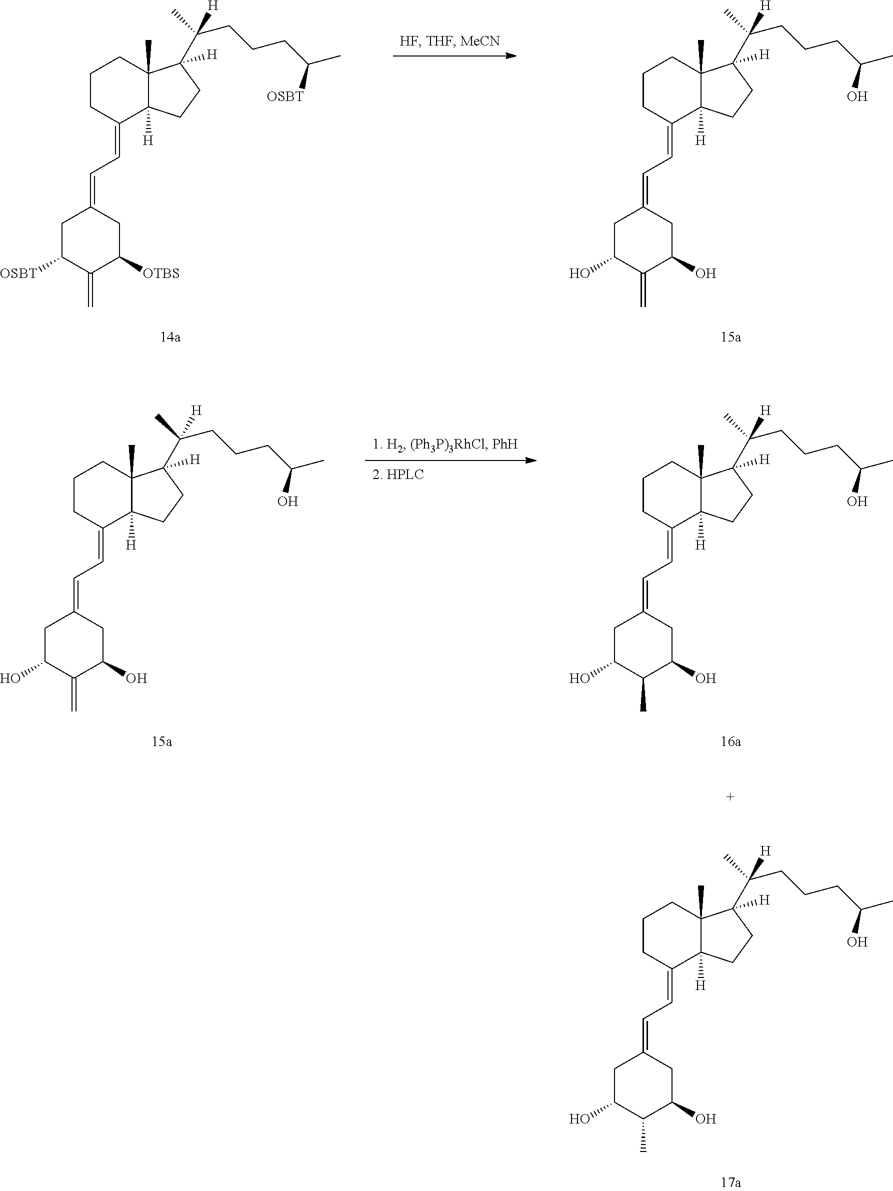

### Example 3

### Preparation of (20S,25S)-2α-methyl-19,26-dinor-1α,25-dihydroxyvitamin D₃ (TA-2) and (20S,25S)-2β-methyl-19,26-dinor-1α,25-dihydroxyvitamin D₃ (IB-1). See Schemes 7-9

### Preparation of (3S)-1-p-Toluenesulfonyloxy-3-triethylsilyloxy-butane (2b)

To a stirred, solution of the(S)-(+)-1,3-butanediol 1b (1 g, 11.1 mmol), DMAP (30 mg, 0.25 mmol) and Et₃N (4.6 mL, 3.33 g, 33 mmol) in anhydrous methylene chloride (20 mL) p-toluenesulfonyl chloride (2.54 g, 13.3 mmol) was added at 0° C. The reaction mixture was stirred at 4° C. for 22 h. Methylene chloride was added and the mixture was washed with water, dried (Na₂SO₄) and concentrated under reduced pressure. A residue was chromatographed on silica gel with hexane/ethyl acetate (8:2, then 1:1) to afford the tosylate (2.31 g, 85% yield) as a colorless oil.

To a stirred solution of the tosylate (2.31 g, 9.5 mmol) and 2,6-lutidine (1.2 mL, 1.12 g, 10.5 mmol) in anhydrous methylene chloride (15 mL) triethylsilyl trifluoromethanesulfonate (2.1 mL, 2.51 g, 9.5 mmol) was added at −50° C. The reaction mixture was allowed to warm to room temperature (4 h) and stirring was continued for additional 20 h. Methylene chloride was added and the mixture was washed with water, dried (Na₂SO₄) and concentrated under reduced pressure. A residue was chromatographed on silica gel with hexane/ethyl acetate (97:3) to afford the product 2b (2.71 g, 80% yield) as a colorless oil:

[α]_{D}+18.0 (c 2.38, CHCl₃); ¹H NMR (400 MHz, CDCl₃) δ 7.77 (2H, d, J=8.2 Hz, o-H_{Ts}), 7.33 (2H, d, J=8.2 Hz, m-H_{Ts}), 4.10 (2H, t, J=6.1 Hz, 1-H₂), 3.90 (1H, m, 3-H), 2.43 (3H, s, Me_{Ts}), 1.72 (2H, m, 2-H₂), 1.10 (3H, d, J=6.2 Hz, 4-H₃), 0.88 (9H, t, J=8.0 Hz; 3×SiCH₂CH₃), 0.50 (6H, q, J=8.0 Hz, 3×SiCH₂CH₃); ¹³C NMR (100 MHz) δ 144.62 (s, p-C_{Ts}), 133.03 (s, i-C_{Ts}), 129.72 (d, m-C_{Ts}), 127.82 (d, o-C_{Ts}), 67.78 (t, C—), 64.46 (d, C-3), 38.47 (t, C-2), 23.82 (q, C-4), 21.52 (q, Me_{Ts}), 6.71 (q, SiCH₂CH₃), 4.77 (t, SiCCH₂CH₃); MS (EI) m/z 359 (5, MH⁺), 329, (87, M⁺-C₂H₅), 259 (100), 233 (54), 197 (50), 17 (74), 163 (40), 149 (48), 135 (38), 115 (53), 91 (71); exact mass calculated for C₁₅H₂₅O₄SSi (M⁺-C₂H₅) 329.1243; found 329.1239.

### Preparation of (3S)-1-Iodo-3-triethylsilyloxy-butane (3b)

To a stirred solution of the tosylate 2b (2.71 g, 7.6 mmol) in anhydrous acetone (50 mL) potassium iodide (8 g, 48 mmol) was added and the reaction mixture was refluxed for 10 h. Water (30 mL) was added and the solution was extracted with ethyl acetate. The combined organic phases were dried (Na₂SO₄) and concentrated under reduced pressure. The residue was chromatographed on silica gel with hexane/ethyl acetate (97:3) to give the alcohol 3b (2.26 g, 95% yield) as a colorless oil:

[α]_{D}+36.3 (c 2.12, CHCl₃); ¹H NMR (400 MHz, CDCl₃) δ 3.89 (14, m, 3-H), 3.22 (2H, t, J=7.0 Hz, 1-H₂) 1.91 (2H, m, 2-H₂), 1.16 (3H, d, J=6.1 Hz, 4-H₃), 0.96 (9H, t, J=7.9 Hz, 3×SiCH₂CH₃), 0.61 (6H, q, J=7.9 Hz, 3×SiC₂CH₃); ¹³C NMR (100 MHz) δ 68.13 (d, C-3), 43.23 (t, C-2), 23.45 (q, C-4), 6.86 (q, SiCH₂CH₃), 4.99 (t, SiCH₂CH₃), 3.34 (t, C-1); MS (EI) m/z 314 (1, M⁺), 299 (1, M⁺-CH₃), 285 (100, M⁺-C₂H₅), 257 (97, M⁺-C₄H₉), 228 (51), 212 (98), 184 (58), 157 (62), 129 (33), 115 (31); exact mass calculated for C₈H₁₈OISi (M⁺-C₂H₅) 285.0172, found 285.0169.

### Preparation of (3S)-Hydroxybutyl-triphenylphosphonium iodide (4b)

To a stirred solution of the iodide 3b (1.67 g, 5.3 mmol) in acetonitrile (50 mL) triphenylphosphine (4.2 g, 16 mmol) was added and the reaction mixture was refluxed for 2 days. Acetonitrile was evaporated under reduced pressure, ethyl acetate (50 mL) was added and the mixture was stirred at room temperature for 4 h. After removal of the solvent by filtration the solid was washed with ethyl acetate, filtered off and dried. The pure phosphonium salt 4b (2.13 g, 87% yield) was obtained as white crystals:

¹H NMR (400 MHz, CD₃OD) δ 8.00-7.70 (15H, m, H_{Ph}), 3.89 (1H, m, 3-H), 3.48 (2H, m, 1-H₂), 1.73 (2H, m, 2-H₂), 1.19 (3H, d, J=6.2 Hz, 4-H₃); ¹³C NMR (100 MHz) δ 136.42 (d, p-C_{Ph}, 134.99 (d, J_{C-P}=10.1 Hz, m-C_{Ph}), 131.71 (d, J_{C-P}=13.1 Hz, o-C_{Ph}), 120.04 (s, J_{C-P}=86.5 Hz, i-C_{Ph}), 67.94 (d, J_{C-P}=16.2 Hz, C-3), 32.52 (t, J_{C-P}=4.1 Hz, C-2), 23.38 (q, C-4), 19.84 (t, J_{C-P}=53.7 Hz, C-1); exact mass calculated for C₂₂H₂₄OPI (M) 335.1565, found 335.1571.

### Preparation of (8S,20S)-des-A,B-20-(hydroxymethyl)pregnan-8-ol (5b)

Ozone was passed through a solution of vitamin D₂ (3 g, 7.6 mmol) in methanol (250 mL) and pyridine (2.44 g, 2.5 mL, 31 mmol) for 50 min at −78° C. The reaction mixture was then flushed with an oxygen for 15 min to remove the residual ozone and the solution was treated with NaBH₄ (0.75 g, 20 mmol). After 20 min the second portion of NaBH₄ (0.75 g, 20 mmol) was added and the mixture was allowed to warm to room temperature. The third portion of NaBI-4 (0.75 g, 20 mmol) was then added and the reaction mixture was stirred for 18 h. The reaction was quenched with water (40 mL) and the solution was concentrated under reduced pressure. The residue was extracted with ethyl acetate and the combined organic phases were washed with 1M aq. HCl, saturated aq. NaHCO₃, dried (NaSO₄) and concentrated under reduced pressure. The residue was chromatographed on silica gel with hexane/ethyl acetate (75:25) to give the diol 5b (1.21 g, 75% yield) as white crystals:

m.p. 106-108° C.; [α]_{D}+30.2° (c 1.46, CHCl₃); ¹H NMR (400 MHz, CDCl₃) δ 4.08 (1H, d, J=2.0 Hz, 8α-H), 3.63 (1H, dd, J=10.5, 3.1 Hz, 22-H), 3.38 (1H, dd, J=10.5, 6.8 Hz, 22-H), 1.99 (1H, br.d, J=13.2 Hz), 1.03 (3H, d, J=6.6 Hz, 21-H₃), 0.956 (3H, s, 18-H₃); ¹³C NMR (100 MHz) δ 9.16 (d, C-8), 67.74 (t, C-22), 52.90 (d), 52.33 (d), 41.83 (s, C-13), 40.19 (t), 38.20 (d), 33.53 (t), 26.62 (t), 22.54 (t), 17.36 (t), 16.59 (q, C-21), 13.54 (q, C-18); MS (EI) m/z 212 (2, M⁺), 194 (34, M⁺-H₂O), 179 (33, M⁺-H₂O—CH₁), 163 (18, M⁺-CH₂OH—H₂O), 135 (36), 125 (54), 111 (100), 95 (63), 81 (67); exact mass calculated for C₁₃H₂₂O (M⁺-H₂O) 194.1671, found 194.1665.

### Preparation of (8S,20S)-des-A,B-8-benzoyloxy-20-(hydroxymethyl)pregnane (6b)

Benzoyl chloride (2.4 g, 2 mL, 17 mmol) was added to a solution of the diol 5b (1.2 g, 5.7 mmol) and DMAP (30 mg, 0.2 mmol) in anhydrous pyridine (20 mL) at 0° C. The reaction mixture was stirred at 4° C. for 24 h, diluted with methylene chloride (100 mL), washed with 5% aq. HCl, water, saturated aq. NaHCO₃, dried (Na₂SO₄) and concentrated under reduced pressure. The residue (3.39 g) was treated with a solution of KOH (1 g, 15.5 mmol) in anhydrous ethanol (30 mL) at room temperature. After stirring of the reaction mixture for 3 h, ice and 5% aq. HCl were added until pH=6. The solution was extracted with ethyl acetate (3×50 mL) and the combined organic phases were washed with saturated aq. NaHCO₃, dried (Na₂SO₄) and concentrated under reduced pressure. The residue was chromatographed on silica gel with hexane/ethyl acetate (75:25) to give the alcohol 6b (1.67 g, 93% yield) as a colorless oil:

[α]_{D}+56.0 (c 0.48, CHCl₃); ¹H NMR (400 MHz, CDCl₃+TMS) δ 8.08-8.02 (2H, m, o-H_{Bz}), 7.59-7.53 (H, m, p-H_{Bz}), 7.50-7.40 (2H, m, m-H_{Bz}), 5.42 (1H, d, J=2.4 Hz, 8α-H), 3.65 (1H, dd, J=10.5, 3.2 Hz, 22-H), 3.39 (1H, dd, J=10.5, 6.8 Hz, 22-H), 1.08 (3H, d, J=5.3 Hz, 21-H₃), 1.07 (3H, s, 18-H₃); ¹³C NMR (125 MHz) δ 166.70 (s, C═O), 132.93 (d, p-C_{Bz}, 130.04 (s, i-C_{Bz}), 129.75 (d, o-C_{Bz}), 128.57 (d, m-C_{Bz}), 72.27 (dd, C-8), 67.95 (t, C-22), 52.96 (d), 51.60 (d), 42.15 (s, C-13), 39.98 (t), 38.61 (d), 30.73 (t), 26.81 (t), 22.91 (t), 18.20 (t), 16.87 (q, C-21), 13.81 (q, C-18); MS (EI) m/z 316 (5, M⁺), 301 (3, M⁺-Me), 299 (1, M⁺-OH), 298 (2, M⁺-H₂O), 285 (10, M⁺-CH₂OH), 257 (6), 230 (9), 194 (80), 135 (84), 105 (100); exact mass calculated for C₂₀H₂₈O₃ 316.2038, found 316.2019.

### Preparation of (8S,20S)-des-A,B-8-benzoyloxy-20-formylpregnane (7)

Sulfur trioxide pyridine complex (1.94 g, 12.2 mmol) as added to a solution of the alcohol 6b (640 mg, 2.03 mmol), triethylamine (1.41 mL, 1.02 g, 10.1 mmol) in anhydrous methylene chloride (10 mL) and anhydrous DMSO (2 mL) at 0° C. The reaction mixture was stirred under argon at 0° C. for 1 h and then concentrated. The residue was diluted with ethyl acetate, washed with brine, dried (Na₂SO₄) and concentrated. The residue was purified by column chromatography on silica gel with hexane/ethyl acetate (95:5) to give the aldehyde 7b (529 mg, 83% yield) as an oil:

¹H NMR (400 MHz, CDCl₃+TMS) δ 9.60 (1H, d, J=3.1 Hz, CHO), 8.05 (2H, m, o-H_{Bz}), 7.57 (1H, m, p-H_{Bz}), 7.45 (2H, m, m-H_{Bz}), 5.44 (1H, s, 8α-H), 2.39 (1H, m, 20-H), 2.03 (2H, dm, J=11.5 Hz), 1.15 (3H, d, J=6.9 Hz, 21-H₃), 1.10 (3H, s, 18-H₃); ¹³C NMR (100 MHz) δ 204.78 (d, CHO), 166.70 (s, C═O), 132.78 (d, p-Bz), 130.69 (s, i-Bz), 129.50 (d, o-Bz), 128.38, (d, m-Bz), 71.66 (d, C-8), 51.30 (d), 50.95 (d), 49.20 (d), 42.38 (s, C-13), 39.62 (t), 30.47 (t), 25.99 (t), 22.92 (t), 17.92 (t), 13.90 (q), 13.35 (q); MS (EI) m/z 314 (1, M⁺), 299 (0.5, M⁺-Me), 286 (1, M⁺-CO), 285 (5, M⁺-CHO), 257 (1, M⁺-C₃H₅O), 209 (10, M⁺-PhCO), 192 (38), 134 (60), 105 (100), 77 (50); exact mass calculated for C₂₀H₂₆O₃ 314.1882, found 314.1887.

### Preparation of (8S,20R)-des-A,B-8-benzoyloxy-20-(hydroxymethyl)pregnane (8b)

The aldehyde 7b (364 mg, 1.12 mmol) was dissolved in methylene chloride (15 mL) and a 40% aq. n-Bu₄NOH solution (1.47 mL, 1.45 g, 2.24 mmol) was added. The resulting mixture was stirred under argon at room temperature for 16 h, diluted with methylene chloride (20 mL), washed with water, dried (Na₂SO₄) and concentrated under reduced pressure. A residue was chromatographed on silica gel with hexane/ethyl acetate (95:5) to afford a mixture of aldehyde 7b and its 20-epimer (292 mg, 80% yield) in ca. 1:2 ratio (by ¹H NMR).

This mixture of aldehydes (292 mg, 0.9 mmol), was dissolved in THF (5 mL) and NaBH₄ (64 mg, 1.7 mmol) was added, followed by a dropwise addition of ethanol (5 mL). The reaction mixture was stirred at room temperature for 30 min and it was quenched with a saturated aq. NH₄Cl solution. The mixture was extracted with ether (3×20 mL) and the combined organic phase was washed with water, dried (Na₂SO₄) and concentrated under reduced pressure. The residue was chromatographed on silica gel with hexane/ethyl acetate (96:4→80:20) to give the desired, pure (20R)-alcohol 8b (160 mg, 55% yield) as an oil and a mixture of 8b and its 20-epimer 6b (126 mg, 43% yield) in ca. 1:3 ratio (by ¹H NMR).

[α]_{D}+50.1 (c 1.19 CHCl₃); ¹H NMR (400 MHz, CDCl₃+TMS) δ 8.05 (2H, m, o-H_{Bz}), 7.55 (1H, m, p-H_{Bz}), 7.44 (2H, m, m-H_{BZ}), 5.41 (1H, s, 8α-H), 3.77 (1H, dd, J=10.4, 3.3 Hz, 22-H), 3.45 (1H, dd, J=10.4, 7.4 Hz, 22-H), 1.067 (3H, s, 18-H₃), 0.973 (3H, d, J=6.6 Hz, 21-H₃); ¹³C NMR (100 MHz) δ 166.36 (s, C═O), 132.61 (d, p-C_{Bz}), 130.63 (s, i-C_{Bz}), 129.39 (d, o-CB), 128.23 (d, m-C_{Bz}), 71.97 (d, C-8), 66.42 (t, C-22), 52.65 (d), 51.38 (d), 41.58 (s, C-13), 39.16 (t), 37.45 (d), 30.38 (t), 26.29 (t), 22.35 (t), 17.89 (t), 16.42 (q, C-21), 13.78 (q, C-18); MS (EI) m/z 316 (16, M⁺), 301 (5, M⁺-Me), 299 (2, M⁺-OH), 298 (3, M⁺-H₂O), 285 (9, M⁺-CH₂OH), 257 (5), 242 (11), 230 (8), 194 (60), 147 (71), 105 (100); exact mass calculated for C₂₀H₂₈O₃ 316.2038, found 316.2050.

### Preparation of (8S,20R)-des-A,B-8-benzoyloxy-20-formylpregnane (9)

Sulfur trioxide pyridine complex (258 mg, 1.62 mmol) was added to a solution of the alcohol 8b (85 mg, 0.27 mmol), triethylamine (188 μL, 136 mg, 1.35 mmol) in anhydrous methylene chloride (5 mL) and anhydrous DMSO (1 mL) at 0° C. The reaction mixture was stirred under argon at 0° C. for 1 h and then concentrated. The residue was diluted with ethyl acetate, washed with brine, dried (Na₂SO₄) and concentrated. The residue was purified by column chromatography on silica gel with hexane/ethyl acetate (95:5) to give the aldehyde 9b (70 mg, 83% yield) as an oil:

[α]_{D}+28.8 (c 0.88, CHCl₃); ¹H NMR (500 MHz, CHO) δ 9.55 (1H, d, J=5.0 Hz, CHO), 8.02 (2H, m, o-H_{Bz}), 7.54 (1H, m, p-H_{Bz}), 7.43 (2H, m, m-H_{Bz}), 5.42 (1H, s, 8α-H), 2.35 (1H, m, 20-H), 2.07 (1H, m), 1.87 (1H, m), 1.05 (3H, s, 18-H₃), 1.04 (3H, d, J=7.8 Hz, 21-H₃); ¹³C NMR (125 MHz) δ 205.51 (d, CHO), 166.34 (s, C═O), 132.76 (d, p-C_{Bz}), 130.62 (s, i-C_{Bz}), 129.47 (d, o-C_{Bz}), 128.35, (d, m-C_{Bz}), 71.52 (d, C-8), 52.08 (d), 51.08 (d) 48.40 (d), 41.55 (s, C-13), 38.54 (t), 30.41 (t), 25.28 (t), 22.08 (t), 17.68 (t), 14.49 (q), 13.38 (q); MS (EI) m/z 314 (2, M⁺), 285 (3, M⁺-CHO), 209 (8, M⁺-PhCO), 192 (30, M⁺-PhCOOH), 177 (14), 134 (45), 105 (100), 77 (50), exact mass calculated for C₁₉H₂₅O₂ (M⁺-CHO) 285.1855, found 285.1849.

### Preparation of (8S,20S)-des-A,B-8-benzoyloxy-20-[(4S)-hydroxy-pent-(1E)-en-yl]pregnane (10b)

To a stirred suspension of the phosphonium salt 4b (201 mg, 0.6 mmol) in anhydrous THF (5 mL) butyllithium (1.6 M, 560 μL, 0.9 mmol) was added at −20° C. The solution turned deep orange. After 1 h a precooled (−20° C.) solution of the aldehyde 9b (65 mg, 0.2 mmol) in anhydrous THF (2 mL) was added and the reaction mixture was stirred at −20° C. for 3 h and at room temperature for 18 h. The reaction was quenched with water and the mixture was extracted with ethyl acetate. Combined organic phases were washed with brine, dried (Na₂SO₄) and evaporated. The residue was chromatographed on silica gel with hexane/ethyl acetate (95:5) to give the product 10b (37 mg, 50% yield):

[α]_{D}−11.4 (c 1.4, CHCl₃); ¹H NMR (400 MHz, CDCl₃+TMS) δ 8.05 (2H, m, o-H_{Bz}), 7.55 (1H, m, p-H_{Bz}), 7.44 (2H, m, m-H_{Bz}), 5.41 (1H, s, 8α-H), 5.45-5.25 (2H, m, 22-H and 23-H), 3.81 (1H, m, 25-H), 1.20 (3H, d, J=6.1 Hz, 27-H₃), 1.04 (3H, s, 18-H₃), 0.94 (3H, d, J=6.6 Hz, 21-H₃); ¹³C NMR (100 MHz) δ 166.45 (s, C═O), 141.11 (d, C-22), 132.66 (d, p-C_{Bz}), 130.87 (s, i-C_{Bz}), 129.53 (d, o-C_{Bz}), 128.32 (d, m-C_{Bz}), 123.41 (d, C-23), 72.09 (d, C-8), 67.23 (d, C-25), 56.34 (d), 51.47 (d), 42.56 (t), 41.95 (s, C-13) 40.15 (d), 39.37 (t), 30.59 (t), 26.80 (t), 22.73 (q, C-27), 22.49 (t), 21.56 (q, C-21), 17.83 (t), 13.85 (q, C-18); MS (EI) m/z 370 (8, M⁺), 355 (0.5, M⁺-CH₃), 326 (2, M⁺-C₂H₄O), 284 (12, M⁺-C₅H₁₀O), 265 (2, M⁺-PhCO) 248 (28, M⁺-PhCOOH), 230 (9), 204 (17), 189 (10), 162 (63), 135 (71), 105 (100); exact mass calculated for C₂₄H₃₄O₃Na (MNa⁺) 393.2406, found 393.2410.

### Preparation of (8S,20S)-des-A,B-8-benzoyloxy-20-[(4S)-hydroxy-pentyl]pregnane (11b)

A solution of the compound 10b (37 mg, 0.1 mmol) in methanol (6 mL) was hydrogenated for 17 h in the presence of 10% palladium on powdered charcoal (6 mg). The reaction mixture was filtered through a bed of Celite with several methanol washes, the filtrate was concentrated and the residue was chromatographed on silica gel with hexane/ethyl acetate (95:5) to give the product 11b (24 mg, 65% yield):

[α]_{D}+32.6 (c 0.9, CHCl₃); ¹H NMR (400 MHz, CDCl₃+TMS) δ 8.06 (2H, m, o-H_{Bz}), 7.56 (1H, m, p-H_{Bz}), 7.45 (2H, m, m-H_{Bz}), 5.41 (1H, s, 8α-H), 3.81 (1H, m, 25-H), 2.02 (2H, m), 1.83 (2H, m), 1.20 (3H, d, J=6.1 Hz, 27-H₃), 1.05 (3H, s, 18-H₃), 0.85 (3H, d, J=6.6 Hz, 21-H₃); ¹³C NMR (100 MHz) δ 166.47 (s, C═O), 132.66 (d, p-C_{Bz}), 130.86 (s, i-C_{Bz}), 129.53 (d, o-C_{Bz}), 128.32 (d, m-C_{Bz}), 72.23 (d, C-8), 68.25 (d, C-25), 55.96 (d), 51.64 (d), 41.95 (s, C-13), 39.85 (t), 39.71 (t), 35.24 (t), 34.84 (d), 30.54 (t), 26.94 (t), 23.51 (q, C-27), 22.52 (t), 22.39 (t), 18.47 (q, C-21), 18.06 (t), 13.83 (q, C-18); MS (EI) m/z 372 (8, M⁺), 354 (2, M⁺-H₂O), 327 (0.5, M⁺-C₂ HO₅), 285 (1, M⁺-C₅H₁₁O), 267 (4, M⁺-PhCO), 250 (59, M⁺-PhCOOH), 232 (18), 163 (23), 135 (64), 105 (100); exact mass calculated for C₂₄H₃₆O₃Na (MNa⁺) 395.2562, found 395.2558.

### Preparation of (8S,20S)-des-A,B-8-benzoyloxy-20-[(4S)-tert-butyldimethylsilyloxy-pentyl]pregnane (2b)

tert-Butyldimethylsilyl trifluoromethanesulfonate (30 μL, 34 mg, 0.13 mmol) was added to a solution of the alcohol 11b (24 mg, 65 μmol) and 2,6-lutidine (30 μL, 28 mg, 0.26 mmol) in anhydrous methylene chloride (3 mL) at −20° C. The mixture was stirred under argon at 0° C. for 1 h. The reaction was quenched with water and extracted with methylene chloride. The combined organic phases were washed with brine, dried (Na₂SO₄) and concentrated under reduced pressure. The residue was chromatographed on silica gel with hexane and hexane/ethyl acetate (97:3) to give the product 12b (32 mg, 100%):

[α]_{D}+25.0 (c 0.55, CHCl₃); ¹H NMR (400 MHz, CDCl₃) δ 8.06 (2H, m, o-H_{Bz}), 7.56 (1H, m, p-H_{Bz}), 7.45 (2H, m, m-H_{Bz}), 5.41 (1H, s, 8α-H), 3.77 (1H, m, 25-H), 2.02 (2H, m), 2.02 (2H, m), 1.82 (2H, m), 1.13 (3H, d, J=6.0 Hz, 27-H₃), 1.04 (3H, s, 18-H₃), 0.90 (9H, s, Si-t-Bu) 0.83 (3H, d, J=6.5 Hz, 21-H), 0.06 (6H, s, SiMe₂); ¹³C NMR (100 MHz) δ 166.50 (s, C═O), 132.66 (d, p-C_{Bz}), 130.91 (s, i-C_{Bz}), 129.55 (d, o-C_{Bz}), 128.33 (d, m-C_{Bz}), 72.27 (d, C-8), 68.81 (d, C-25), 55.99 (d), 51.67 (d), 41.96 (s, C-13), 40.21 (t), 39.84 (t), 35.37 (t), 34.86 (d), 30.58 (t), 26.95 (t), 25.92 (q, SiCMe₃), 23.88 (q, C-27), 22.55 (t), 22.46 (t), 18.48 (q, C-21), 18.17 (s, SiCMe₃), 18.05 (t), 13.79 (q, C-18), −4.39 (q, SiMe), −4.68 (q, SiMe); MS (EI) m/z 485 (1, M⁺-H), 471 (1, M⁺-CH₃), 307 (9, M⁺-PhCOOH—C₄H₉), 233 (71, M⁺-PhCOOH-t-BuSiMe₂O), 197 (71), 179 (92), 163 (81), 135 (71), 105 (100); exact mass calculated for C₃₀H₅₀O₃SiNa (MNa⁺) 509.3427, found 509.3446.

### Preparation of (8S,20S)-des-A,B-20-[(4S)-tert-butyldimethylsilyloxy-pentyl]pregnan-8-ol (13b)

A solution of sodium hydroxide in ethanol (2.5M, 2 mL) was added to a stirred solution of the benzoate 12b (31 mg, 64 μmol) in anhydrous ethanol (10 mL) and the reaction mixture was refluxed for 18 h. The mixture was cooled to room temperature, neutralized with 5% aq. HCl and extracted with dichloromethane. Combined organic phases were washed with saturated aq. NaHCO₃, dried (Na₂SO₄) and evaporated. The residue was chromatographed on silica gel with hexane/ethyl acetate (95:5) to give the alcohol 13b (18 mg, 74% yield):

[α]_{D}+14.3 (c 0.8, CHCl₃); ¹H NMR (500 MHz, CDCl₃) δ 4.07 (1H, d, J=2.3 Hz, 8α-H), 3.75 (1H, m, 25-H), 1.97 (1H, m), 1.80 (3H, m), 1.11 (3H, d, J=6.1 Hz, 27-H₃), 0.92 (3H, s, 18H₃); 0.88 (9H, s, Si-t-Bu), 0.81 (3H, d, J=6.6 Hz, 21-H₃), 0.04 (6H, s, SiMe₂); ¹³C NMR (125 MHz), δ 69.44 (d, C-8), 68.80 (d, C-25), 56.21 (d), 52.65 (d), 41.88 (s, C-13), 40.29 (t), 40.20 (t), 35.29 (t), 34.76 (d), 33.57 (t), 27.07 (t), 25.91 (q, SiCMe₃), 23.88 (q, C-27), 22.46 (t), 22.41 (t), 18.49 (q, C-21), 18.17 (s, SiCMe₃), 17.45 (t), 13.76 (q, C-18), −4.40 (q, SiMe), −4.70 (q, SiMe); MS (EI) m/z 382 (2, M⁺), 367 (4, M⁺-CH₃), 325 (8, M⁺-C₄H₉), 307 (3, M⁺-C₄H₉—H₂O), 233 (73), 191 (53), 177 (89), 163 (86) 149 (66), 135 (98), 123 (75), 109 (93), 97 (100); exact mass calculated for C₁₉H₃₇O₂Si (M⁺-C₄H₉) 325.2563, found 325.2567.

### Preparation of (20S)-des-A,B-20-[(4S)-tert-butyldimethylsilyloxy-pentyl]pregnan-8-one (14b)

Molecular sieves A4 (50 mg) were added to a solution 4-methylmorpholine N-oxide (17 mg, 0.17 mmol) in dichloromethane (0.5 mL). The mixture was stirred at room temperature for 15 min and tetrapropylammonium perruthenate (2 mg, 6 μmol) was added, followed by a solution of alcohol 13b (18 mg, 47 μmol) in dichloromethane (300+300 μL). The resulting suspension was stirred at room temperature for 1 h. The reaction mixture was filtered through a Waters silica Sep-Pak cartridge (5 g) that was further washed with dichloromethane. After removal of the solvent the ketone 14b (17 mg, 95% yield) was obtained as a colorless oil:

[α]_{D}−20.2 (c 0.75, CHCl₃); ¹H NMR (400 MHz, CDCl₃) δ 3.76 (1H, m, 25-H), 2.44 (1H, dd, J=11.4, 7.7 Hz), 1.12 (3H, d, J=6.1 Hz, 27-H₃), 0.89 (9H, s, Si-t-Bu), 0.84 (3H, d, J=5.9 Hz, 21-H₃), 0.63 (3H, s, 18-H₃), 0.05 (6H, s, SiMe₂); ¹³C NMR (100 MHz) δ 212.12 (s), 68.73 (d, C-25), 62.02 (d), 56.18 (d), 49.93 (s, C-13), 40.96 (t), 40.14 (t), 38.85 (t), 35.54 (t), 34.86 (d), 27.16 (t), 25.90 (q, SiCMe₃), 24.03 (t), 23.89 (q, C-27), 22.42 (t), 18.93 (t), 18.44 (q, C-21), 18.15 (s, SiCMe₃), 12.70 (q, C-18), −4.38 (q, SiMe), −4.69 (q, SiMe), MS (EI) m/z 380 (2, M⁺), 379 (3, M⁺-H), 365 (14, M⁺-CH₃), 324 (60, M⁺-C₄H₈), 267 (17), 253 (28), 231 (59), 189 (61), 161 (54), 135 (76), 95 (90), 75 (100); exact mass calculated for C₁₉H₃₅O₂Si (M⁺-C₄H₉) 323.2406, found 323.2405.

### Preparation of (20S,25S)-2-Methylene-19,26-dinor-1α,25-dihydroxyvitamin D₃ (17b)

To a solution of phosphine oxide 15b (74 mg, 127 μmol) in anhydrous THF (500 μL) at −20° C. was slowly added PhLi (1.8 M in di-n-butylether, 105 μL, 189 μmol) under argon with stirring. The solution turned deep orange. After 30 min the mixture was cooled to −78° C. and a precooled (−78° C.) solution of ketone 14b (16 mg, 42 μmol) in anhydrous THF (200+100 μL) was slowly added. The mixture was stirred under argon at −78° C. for 3 h and at 0° C. for 18 h. Ethyl acetate was added, and the organic phase was washed with brine, dried (Na₂SO₄) and evaporated. The residue was dissolved in hexane and applied on a Waters silica Sep-Pak cartridge (2 g). The cartridge was washed with hexane and hexane/ethyl acetate (99.5:0.5) to give 19-norvitamin derivative 16b (25 mg, 80% yield). Then the Sep-Pak was washed with ethyl acetate to recover diphenylphosphine oxide 15b (40 mg). For analytical purpose a sample of the protected vitamin 16b was further purified by HPLC (9.4×250 mm Zorbax Sil column, 4 mL/min, hexane/2-propanol (99.9:0.1) solvent system, Rₜ=3.51 min):

UV (in hexane) λₘₐₓ 262.6, 253.2, 244.8 nm; ¹H NMR (400 MHz, CDCl₃) δ 6.23 and 5.85 (each 1H, each d, J=11.1, Hz, 6- and 7-H), 4.98 and 4.93 (each 1H, each s, ═CH₂), 4.42 (2H, m, 1β- and 3α-H), 3.77 (1H, m, 25-H), 2.83 (1H, dm, J=11.8 Hz, 9β-H), 2.52 (1H, dd, J=13.3, 5.9 Hz, 10α-H), 2.47 (1H, dd, J=12.4, 4.3 Hz; 4α-H), 2.33 (1H, dm, J=13.3 Hz, 10β-H), 2.19 (1H, dd, J=12.4, 8.5 Hz, 4β-H), 1.12 (3H, d, J=6.0 Hz, 27-H₃), 0.903 (9H, s, Si-t-Bu), 0.897 (9H, s, Si-t-Bu), 0.871 (9H, s, Si-t-Bu), 0.84 (3H, d, J=6.5 Hz, 21-H₃), 0.547 (3H, s, 18-H₃), 0.086 (3H, s, SiMe), 0.072 (3H, s, SiMe), 0.055 (9H, s, 3×SiMe), 0.032 (3H, s, SiMe); ¹³C NMR (100 MHz) δ 152.98 (s, C-2), 141.24 (s, C-8), 132.70 (s, C-5), 122.42 (d, C-6), 116.09 (d, C-7), 106.25 (t, ═CH₂), 72.52 and 71.63 (each d, C-1 and C-3), 68.80 (d, C-25), 56.32 (d), 56.17 (d), 47.60 (t), 45.70 (s, C-13), 40.50 (t), 40.19 (t), 38.55 (t), 35.60 (t), 35.52 (d), 28.76 (t), 27.42 (t), 25.92 (q, SiCMe₃), 25.84 (q, SiCMe₃), 25.78 (q, SiCMe₃), 23.87 (q, C-27), 23.43 (t), 22.55 (t), 22.10 (t), 18.55 (q, C-21), 18.25 (s, SiCMe₃), 18.17 (s, 2×SiCMe₃), 12.30 (q, C-18), −4.39 (q, SiMe), −4.69 (q, SiMe), −4.86 (q, 2×SiMe), −4.91 (q, SiMe), −5.10 (q, SiMe); exact mass calculated for C₄₄H₈₄O₃Si₃Na (MNa⁺) 767.5626, found 767.5621.

The protected vitamin 16b (25 mg, 34 μmol) was dissolved in THF (2 mL) and acetonitrile (2 mL). A solution of aq. 48% HF in acetonitrile (1:9 ratio, 2 mL) as added at 0° C. and the resulting mixture was stirred at room temperature for 6 h, Saturated aq. NaHCO₃ solution was added and the reaction mixture was extracted with ethyl acetate. The combined organic phases were washed with brine, dried (Na₂SO₄) and concentrated under reduced pressure. The residue was diluted with 2 mL of hexane/ethyl acetate (8:2) and applied on a Waters silica Sep-Pak cartridge (2 g). An elution with hexane/ethyl, acetate (8:2) and later with ethyl acetate gave the crude product 17b (14 mg). The vitamin 17b was further purified by reverse phase HPLC [9.4×250 mm Zorbax Eclipse XDB-C18 column, 3 mL/min, methanol/water (85:15) solvent system, Rₜ=10.67 min.] to give a colorless oil (11.34 mg, 83% yield):

UV (in EtOH) λₘₐₓ 261.4, 252.2, 244.4 nm; ¹H NMR (500 MHz, CDCl₃) δ 6.35 and 5.88 (1H and 1H, each d, J=11.2 Hz, 6- and 7-H), 5.10 and 5.08 (each 1H, each s, ═CH₂), 4.47 (2H, m, 1β- and 3α-H), 3.78 (1H, m, 25-H), 2.84 (1H, dd, J=13.1, 4.4 Hz, 10β-H), 2.81 (1H, br d, J=11.9 Hz, 9β-H), 2.56 (1H, dd, J=13.4, 3.6 Hz, 4α-H), 2.32 (1H, dd, J=13.4, 6.1 Hz, 4β-H), 2.28 (1H, dd, J=13.1, 8.4 Hz, 10α-H), 1.18 (3H, d, J=6.2 Hz, 27-H₃), 0.84 (3H, d, J=6.5 Hz, 21-H₃), 0.543 (3H, s, 18-H₃); ¹³C NMR (125 MHz) δ 151.98 (s, C-2), 143.35 (s, C-8), 130.43 (s, C-5), 124.22 (d, C-6), 115.31 (d, C-7), 107.70 (t, ═CH₂), 71.79 and 70.66 (each d, C-1 and C-3), 68.29 (d, C-25), 56.33 (d), 56.13 (d), 45.80 (t), 45.80 (s, C-13), 40.34 (t), 39.74 (t), 38.14 (t), 35.5 (t), 35.41 (d), 28.94 (t), 27.28 (t), 23.48 (t), 23.48 (q, C-27), 22.43 (t), 22.14 (t), 18.52 (q, C-21), 12.36 (q, C-18); MS (EI) m/z 402 (100, M⁺), 384 (3, M⁺-H₂O), 369 (2, M⁺-H₂O—CH₃), 351 (2, M⁺-2H₂O—CH₃), 287 (6, M⁺-C₇H₁₅O), 269 (14), 251 (15), 192 (12), 161 (16), 147 (48), 135 (69), 95 (68); exact mass calculated for C₂₆H₄₂O₃ (M⁺) 402.3134, found 402.3147.

### Preparation of (20S,25S)-2α-methyl-19,26-dinor-1α,25-dihydroxyvitamin D₃ (18b) and (20S,25S)-2β-methyl-19,26-dinor-1α,25-dihydroxyvitamin D₃ (19b)

Tris(triphenylphosphine)rhodium (I) chloride (8 mg, 8.6 μmol) was added to dry benzene (5 mL) presaturated with hydrogen (15 min). The mixture was stirred at room temperature until a homogeneous solution was formed (ca. 25 min). A solution of vitamin 17b, (2.6 mg, 6.5 μmol) in dry benzene (3 mL) was then added and the reaction was allowed to proceed under a continuous stream of hydrogen for 4 h. Benzene was removed under vacuum, the residue was redissolved in hexane/ethyl acetate (1:1) and applied on a Waters silica Sep-Pak cartridge (2 g). A mixture of 2-methyl vitamins was eluted with the same solvent system. The compounds were further purified by HPLC (9.4×250 mm Zorbax Sil column, 4 mL/min) using hexane/2-propanol (85:15) solvent system. The mixture of 2-methyl-19-norvitamins 18b and 19b gave a single peak at Rₜ=9.3 min. Separation of both epimers was achieved by reversed-phase HPLC (9.4×250 mm Zorbax RX C18 column, 3 mL/min) using methanol/water (85:15) solvent system. 2β-Methyl vitamin 19b (845 μg, 32% yield) was collected at Rₜ=8.2 min. and its 2α-epimer 18b (957 μg, 36% yield) at Rₜ=11.0 min:

2α-Methyl analog 18b UV (in EtOH) λₘₐₓ 260.0, 251.0, 243.5 nm; ¹H NMR (500 MHz, CDCl₃) δ 6.37 and 5.82 (1H and 1H, each d, J=11.3 Hz, 6- and 7-H), 3.96 (1H, m, 1β-H), 3.79 (1H, m, 25-H), 3.61 (1H, m, 3α-H), 2.80 (2H, br m, 9β- and 10α-H), 2.60 (1H, dd, J=12.9, 4.4 Hz, 4α-H), 2.22 (1H, br d, J=13.3 Hz, 10β-H); 2.13 (1H, ˜t, J˜11.2 Hz, 4β-H), 1.190 (3H, d, J=6.2 Hz, 27-H₃), 1.133 (3H, d, J=−6.8 Hz, 2α-CH₃), 0.845 (3H, d, J=6.5 Hz, 21-H₃), 0.532 (3H, s, 18-H₃); MS (EI) m/z 404 (44, M⁺), 386 (17, M⁺-H₂O), 368 (15, M⁺-2H₂O), 350 (25, M⁺-3H₂O), 335 (7, M⁺-3H₂O—CH₃), 317 (10, M⁺-C₅H₁₀OH), 312 (16), 289 (25, M⁺-C₇H₁₄OH), 271 (22, M⁺-C₇H₁₄OH—H₂O), 253 (37), 245 (100), 199 (17), 187 (27), 174 (32), 135 (66); exact mass calculated for C₂₆H₄₄O₃ (M⁺) 404.3290, found 404.3278.

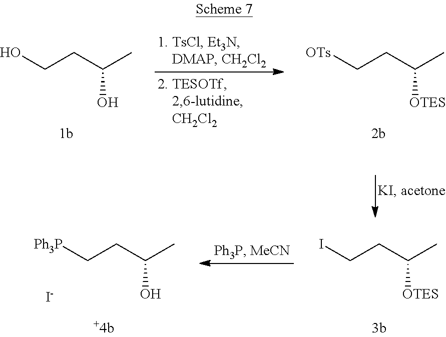

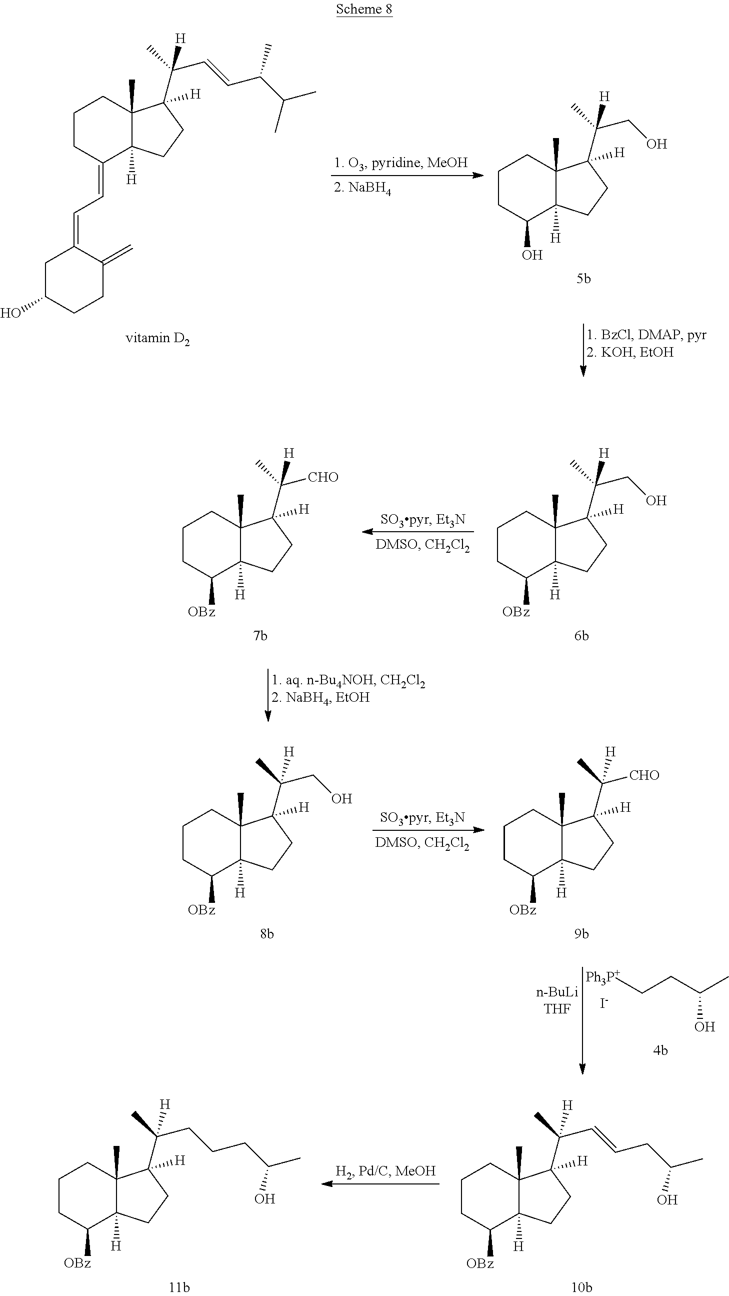

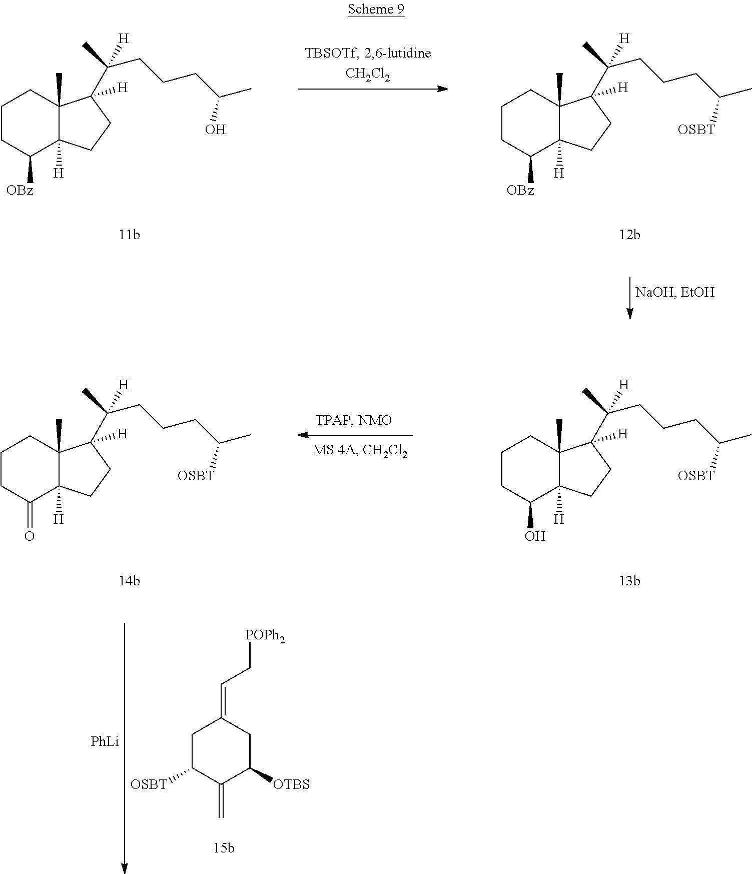

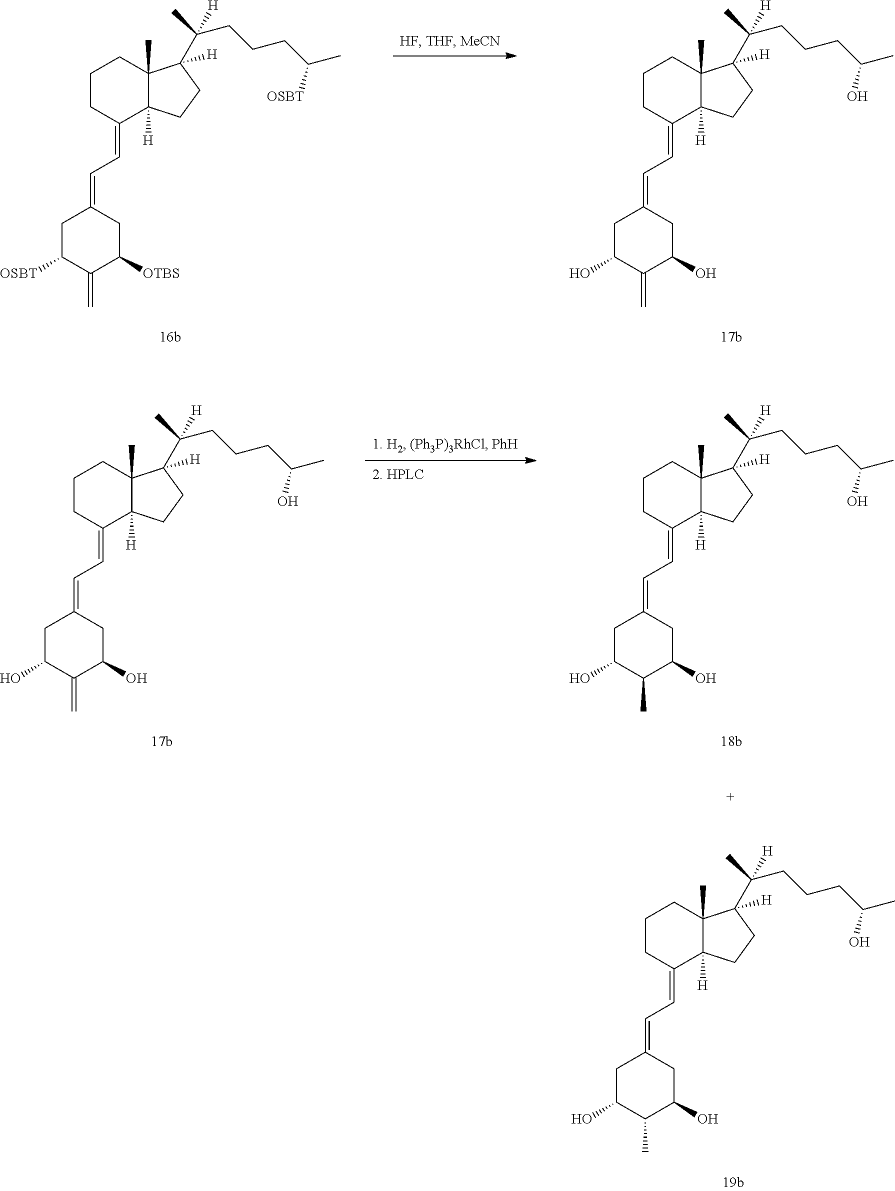

### Example 4

### Preparation of (20R,25S)-2α-methyl-19,26-dinor-1α,25-dihydroxyvitamin D₃ (NC-2) and (20R,25S)-2β-methyl-19,26-dinor-1α,25-dihydroxyvitamin D₃ (TH-1). See Schemes 10-12

### Preparation of (3S)-1-p-Toluenesulfonyloxy-3-triethylsilyloxy-butane (2c)

To a stirred solution of the (S)-(+)-1,3-butanediol 1c (1 g, 11.1 mmol), DMAP (30 mg, 0.25 mmol) and Et₃N (4.6 mL, 3.33 g, 33 mmol) in anhydrous methylene chloride (20 mL) p-toluenesulfonyl chloride (2.54 g, 13.3 mmol) was added at 0° C. The reaction mixture was stirred at 4° C. for 22 h. Methylene chloride was added and the mixture was washed with water, dried (Na₂SO₄) and concentrated under reduced pressure. A residue was chromatographed on silica gel with hexane/ethyl acetate (8:2, then 1:1) to afford the tosylate (2.31 g, 85% yield) as a colorless oil.

To a stirred solution of the tosylate (2.31 g, 9.5 mmol) and 2,6-lutidine (1.2mL, 1.12 g, 10.5 mmol) in anhydrous methylene chloride (15 mL) triethylsilyl trifluoromethanesulfonate (2.1 mL, 2.51 g, 9.5 mmol) was added at −50° C. The reaction mixture was allowed to warm to room temperature (4 h) and stirring was continued for additional 20 h. Methylene chloride was added and the mixture was washed with water, dried (Na₂SO₄) and concentrated under reduced pressure. A residue was chromatographed on silica gel with hexane/ethyl acetate (97:3) to afford the product 2c (2.71 g, 80% yield) as a colorless oil:

[α]_{D}+18.0 (c 2.38, CHCl₃); ¹H NMR (400 MHz, CDCl₃) δ 7.77 (2H, d, J=8.2 Hz, o-H_{Ts}), 7.33 (2H, d, J=8.2 Hz, m-H_{Ts}), 4.10 (2H, t, J=6.1 Hz, 1-H₂), 3.90 (1H, m, 3-H), 2.43 (3H, s, Me_{Ts}), 1.72 (2H, m, 2-H₂), 1.10 (3H, d, J=6.2 Hz, 4-H₃), 0.88 (9H, t, J=8.0 Hz, 3×SiCH₂CH₃), 0.50 (6H, q, J=8.0 Hz, 3×SiCH₂CH₃); ¹³C NMR (100 MHz) δ 144.62 (s, p-C_{Ts}), 133.03 (s, i-C_{Ts}), 129.72 (d, m-C_{Ts}), 127.82 (d, o-C_{Ts}), 67.78 (t, C-1); 64.46 (d, C-3), 38.47 (t, C-2), 23.82 (q, C-4), 21.52 (q, Me_{Ts}), 6.71 (q, SiCH₂CH₃), 4.77 (t, SiCH₂CH₃), MS (EI) m/z 359 (5, MH⁺), 329 (87, M⁺-C₂H₅), 259 (100), 233 (54), 197 (50), 179 (74), 163 (40), 149 (48), 135 (38), 115 (53), 91 (71); exact mass calculated for C₁₅H₂₅O₄SSi (M⁺-C₂H₅) 329.1243, found 329.1239.

### Preparation of (3S)-1-Iodo-3-triethylsilyloxy-butane (3c)

To a stirred solution of the tosylate 2c (2.71 g, 7.6 mmol) in anhydrous acetone (50 mL) potassium iodide (8 g, 48 mmol) was added and the reaction mixture was refluxed for 10 h. Water (30 mL) was added and the solution was extracted with ethyl acetate. The combined organic phases were dried (Na₂SO₄) and concentrated under reduced pressure. The residue was chromatographed on silica gel with hexane/ethyl acetate (97:3) to give the alcohol 3c (2.26 g, 95% yield) as a colorless oil:

[α]_{D}+363 (c 2.12, CHCl₃); ¹H NMR (400 MHz, CDCl₃) δ 3.89 (1H, m, 3-H), 3.22 (2H, t, J=7.0 Hz, 1-H₂), 1.91 (2H, m, 2-H₂), 1.16 (3H, d, J=6.1 Hz, 4-H₃), 0.96 (9H, t, J=7.9 Hz, 3×SiCHCH₃), 0.61 (6H, q, J=7.9 Hz, 3×SiCH₂CH₃); ¹³C NMR (100 MHz) δ 68.13 (d, C-3), 43.23 (t, C-2), 23.45 (q, C-4), 6.86 (q, SiCH₂CH₃), 4.99 (t, SiCH₂CH₃), 3.34 (t, C-1); MS (EI) m/z 314 (1, M⁺), 299 (1, M⁺-CH₃), 285 (100, M⁺-C₂H₅), 257 (97, M⁺-C₄H₉), 228 (51), 212 (98), 184 (58), 157 (62), 129 (33), 115 (31); exact mass calculated for C₈H₁₈OISi (M⁺-C₂H₅) 285.0172, found 285.0169.

### Preparation of (3S)-Hydroxybutyl-triphenylphosphonium iodide (4c)

To a stirred solution of the iodide 3c (1.67 g, 5.3 mmol) in acetonitrile (50 mL) triphenylphosphine (4.2 g, 16 mmol) was added and the reaction mixture was refluxed for 2 days. Acetonitrile was evaporated under reduced pressure, ethyl acetate (50 mL) was added and the mixture was stirred at room temperature for 4 h. After removal of the solvent by filtration the solid was washed with ethyl acetate, filtered off and dried. The pure phosphonium salt 4c (2.13 g, 87% yield) was obtained as white crystals:

¹H NMR (400 MHz, CD₃OD) δ 8.00-7.70 (15H, m, H_{Ph}), 3.89 (1H, m, 3-H), 3.48 (2H, m, 1-H₂), 1.73 (2H, m, 2-H₂), 1.19 (3H, d, J=6.2 Hz, 4-H₃), ¹³C NMR (100 MHz) δ 136.42 (d, p-C_{Ph}), 134.99 (d, J_{C-P}=10.1 Hz, m-C_{Ph}), 131.71 (d, J_{C-P}=13.1 Hz, o-C_{Ph}) 120.04 (s, J_{C-P}=86.5 Hz, i-C_{Ph}), 67.94 (d, J_{C-P}=16.2 Hz, C-3), 32.52 (t, J_{C-P}=4.1 Hz, C-2), 23.38 (q, C-4), 19.84 (t, J_{C-P}=53.7 Hz, C-1); exact mass calculated for C₂₂H₂₄OPI (M⁺) 335.1565, found 335.1571.

### Preparation of (8S,20S)-de-A,B-20-(hydroxymethyl)pregnan-8-ol (5c)

Ozone was passed through a solution of vitamin D₂ (3 g, 7.6 mmol) in methanol (250 mL) and pyridine (2.44 g, 2.5 mL, 31 mmol) for 50 min at −78° C. The reaction mixture was then flushed with an oxygen for 15 min to remove the residual ozone and the solution was treated with NaBH₄ (0.75 g, 20 mmol). After 20 min the second portion of NaBH₄ (0.75 g, 20 mmol) was added and the mixture was allowed to warm to room temperature. The third portion of NaBH₄ (0.75 g, 20 mmol) was then added and the reaction mixture was stirred for 18 h. The reaction was quenched with water (40 mL) and the solution was concentrated under reduced pressure. The residue was extracted with ethyl acetate and the combined organic phases were washed with 1M aq. HCl, saturated aq. NaHCO₃, dried (Na₂SO₄) and concentrated under reduced pressure. The residue was chromatographed on silica gel with hexane/ethyl acetate (75:25) to give the diol 5c (1.21 g, 75% yield) as white crystals:

m.p. 106-108° C.; [α]_{D}+30.2° (c 1.46, CHCl₃); ¹H NMR (400 MHz, CDCl₃) δ 4.08 (1H, d, J=2.0 Hz, 8α-H), 3.63 (1H, dd, J=10.5, 3.1 Hz, 22-H), 3.38 (1H, dd, J=10.5, 6.8 Hz, 22-H), 1.99 (1H, br d, J=13.2 Hz), 1.03 (3H, d, J=6.6 Hz, 21-H₃), 0.956 (3H, s, 18-H₃); ¹³C NMR (100 MHz) δ 69.16 (d, C-8), 67.74 (t, C-22), 52.90 (d), 52.33 (d), 41.83 (s, C-13), 40.19 (t), 38.20 (d), 33.53 (t), 26.62 (t), 22.54 (t), 17.36 (t), 16.59 (q, C-21), 13.54 (q, C-18); MS (EI) m/z 212 (2, M⁺), 194 (34, M⁺-H₂O), 179 (33, M⁺-H₂O—CH₃), 163 (18, M⁺-CH₂OH—H₂O), 135 (36), 125 (54), 111 (100), 95 (63), 81 (67); exact mass calculated for C₁₃H₂₂O (M⁺-H₂O) 194.1671, found 194.1665.

### Preparation of (8S,20S)-de-A,B-8-benzoyloxy-20-(hydroxymethyl)pregnane (6)

Benzoyl chloride (2.4 g, 2 mL, 17 mmol) was added to a solution of the diol 5c (1.2 g, 5.7 mmol) and DMAP (30 mg, 0.2 mmol) in anhydrous pyridine (20 mL) at 0° C. The reaction mixture was stirred at 4° C. for 24 h, diluted with methylene chloride (100 mL), washed with 5%, aq. HCl, water, saturated aq. NaHCO₃, dried (Na₂SO₄) and concentrated under reduced pressure. The residue (3.39 g) was treated with a solution of KOH (1 g, 15.5 mmol) in anhydrous ethanol (30 mL) at room temperature. After stirring of the reaction mixture for 3 h, ice and 5% aq. HCl were added until pH=6. The solution was extracted with ethyl acetate (3×50 mL) and the combined organic phases were washed with saturated aq. NaHCO₃, dried (Na₂SO₄) and concentrated under reduced pressure. The residue was chromatographed on silica gel with hexane/ethyl acetate (75:25) to give the alcohol 6c (1.67 g, 93% yield) as a colorless oil:

[α]_{D}+56.0 (c 0.48, CHCl₃); ¹H NMR (400 MHz, CDCl₃+TMS) δ 8.08-8.02 (2H, m o-H_{Bz}), 7.59-7.53 (1H, m, p-H_{Bz}), 7.50-7.40 (2H, m, m-H_{Bz}), 5.42 (1H, d, J=2.4 Hz, 8α-H), 3.65 (1H, dd, J=10.5, 3.2 Hz, 22-H), 3.39 (1H, dd, J=10.5, 6.8 Hz, 22-H), 1.08 (3H, d, J=5.3 Hz, 21-H₃), 1.07 (3H, s, 18-H₃); ¹³C NMR (125 MHz) δ 166.70 (s, C═O), 132.93 (d, p-C_{Bz}), 131.04 (s, i-C_{Bz}), 129.75 (d, o-C_{Bz}), 128.57 (d, m-C_{Bz}), 72.27 (d, C-8), 67.95 (t, C-22), 52.96 (d), 51.60 (d), 42.15 (s, C-13), 39.98 (t), 38.61 (d), 30.73 (t), 26.81 (t), 22.91 (t), 18.20 (t), 16.87 (q, C-21), 13.81 (q, C-18); MS (EI) m/z 316 (5, M⁺), 301 (3, M⁺-Me) 299 (1, M⁺-OH), 298 (2, M⁺-H₂O), 285 (10, M⁺-CH₂OH), 257 (6), 230 (9), 194 (80), 135 (84), 105 (100); exact mass calculated for C₂₀H₂₈O₃ 316.2038, found 316.2019.

### Preparation of (8S,20S)-de-A,B-8-benzoyloxy-20-formylpregnane (7c)

Sulfur trioxide pyridine complex (1.94 g, 12.2 mmol) was added to a solution of the alcohol 6c (640 mg, 2.03 mmol), triethylamine (1.41 mL, 1.02 g, 10.1 mmol) in anhydrous methylene chloride (10 mL) and anhydrous DMSO (2 mL) at 0° C. The reaction mixture was stirred under argon at 0° C. for 1 h and then concentrated. The residue was diluted with ethyl acetate, washed with brine, dried (Na₂SO₄) and concentrated. The residue was purified by column chromatography on silica gel with hexane/ethyl acetate (95:5) to give the aldehyde 7c (529 mg, 83% yield) as an oil: [α]_{D}+63.1 (c 5.85, CHCl₃); ¹H NMR (400 MHz), CDCl₃+TMS) δ 9.60 (1H, d, J=3.1 Hz, CHO), 8.05 (2H, m, o-H_{Bz}), 7.57 (1H, m, p-H_{Bz}), 7.45 (2H, m, m-H_{Bz}), 5.44 (1H, s, 8α-H), 2.39 (1H, m, 20-H), 2.03 (2H, dm, J=11.5 Hz), 1.15 (3H, d, J=6.9 Hz, 21-H₃), 1.10 (3H, s, 18-H₃); ¹³C NMR (100 MHz) δ 204.78 (d, CHO), 132.78 (d, p-Bz), 130.69 (s, i-Bz), 129.50 (d, o-Bz), 128.38, (d, m-Bz), 71.66 (d, C-8), 51.30 (d), 50.95 (d), 49.20 (d), 42.38 (s, C-13), 39.62 (t), 30.47 (t), 25.99 (t), 22.92 (t), 17.92 (t), 13.90 (q), 13.35 (q); MS (EI) m/z 314 (1, M⁺), 299 (0.5, M⁺-Me), 286 (1, M⁺-CO), 285 (5, M⁺-CHO), 257 (1, M⁺-C₃H₅O), 209 (10, M⁺-PhCO), 192 (38), 134 (60), 105 (100), 77 (50); exact mass calculated for C₂₀H₂₆O₃ 314.1882, found 314.1887.

### Preparation of (8S,20R)-de-A,B-8-benzoyloxy-20-[(4S)-hydroxy-pent-(1E)-en-yl]pregnane (8c)

To a stirred suspension of the phosphonium salt 4c (310 mg, 0.67 mmol) in anhydrous THF (5 mL) butyllithium (1.6 M, 840 μL, 1.34 mmol) was added at −20° C. The solution turned deep orange. After 1 h a precooled (−20° C.) solution of the aldehyde 7c (70 mg, 0.22 mmol) in anhydrous THF (2 mL) was added and the reaction mixture was stirred at −20° C. for 3 h and at room temperature for 18 h. The reaction was quenched with water and the mixture was extracted with ethyl acetate. Combined organic phases were washed with brine, dried (Na₂SO₄) and evaporated. The residue was chromatographed on silica gel with hexane/ethyl: acetate (95:5) to give the product 8c (42 mg, 52% yield):

[α]_{D}+98.7 (c 1.75, CHCl₃); ¹H NMR (400 MHz, CDCl₃+TMS) δ 8.05 (2H, m, o-H_{Bz}), 7.56 (1H, m, p-H_{Bz}), 7.45 (2H, m, m-H_{Bz}), 5.41 (1H, s, 8α-H), 5.40-5.20 (2H, m, 22-H and 23-H), 3.79 (1H, m, 25-H), 1.17 (3H, d, J=6.2 Hz, 27-H₃), 1.07 (3H, s, 18-H₃), 1.05 (3d, d, J=6.7 Hz, 21-H₃); ¹³C NMR (100 MHz) δ 166.43 (s, C═O), 140.86 (d, C-22), 132.66 (d, p-C_{Bz}), 130.82 (s, i-C_{Bz}), 129.50 (d, o-C_{Bz}), 128.32 (d, m-C_{Bz}), 123.42 (d, C-23), 72.12 (d, C-8), 67.15 (d, C-25), 55.87 (d), 51.63 (d), 42.48 (t), 41.81 (s, C-13), 39.93 (d), 39.79 (t), 30.47 (t), 27.65 (t), 22.59 (t), 22.48 (q, C-27), 20.47 (q, C-21), 17.98 (t), 13.72 (q, C-18); MS (EI) m/z 370 (7, M⁺), 352 (0.5, M⁺-H₂O), 326 (2, M⁺-C₂H₄O), 284 (11, M⁺-M C₅H₁₀O), 248 (28, M⁺-PhCOOH), 230 (10), 204 (26), 189 (13), 162 (68), 135 (77), 105 (100); exact mass calculated for C₂₄H₃₄O₃ (M⁺) 370.2508, found 370.2491.

### Preparation of (8S,20R)-de-A,B-8-benzoyloxy-20-[(4S)-hydroxy-pentyl]pregnane (9c)

A solution of the compound 8c (42 mg, 0.11 mmol) in methanol (6 mL) was hydrogenated for 17 h in the presence of 10% palladium on powdered charcoal (7 mg). The reaction mixture was filtered through a bed of Celite with several methanol washes, the filtrate was concentrated and the residue was chromatographed on silica gel with hexane/ethyl acetate (95:5) to give the product 9c (32 mg, 78% yield):

[α]_{D}+72.9 (c 1.4, CHCl₃); ¹H NMR (400 MHz, CDCl₃+TMS), δ 8.05 (2H, m, o-H_{Bz}), 7.55 (1H, m, p-H_{Bz}), 7.44 (2H, m, m-H_{Bz}), 5.41 (1H, s 8α-H), 3.80 (1H, m, 25-H); 2.04 (2H, m), 1.83 (2H, m), 1.19 (3H, d, J=6.2 Hz, 27-H₃), 1.04 (3H, s, 18-H₃), 0.95 (3H, d, J=6.5Hz, 21-H₃) ¹³C NMR (100 MHz) δ 166.47 (s, C═O), 132.64 (d, p-C_{Bz}) 130.86 (s, i-C_{Bz}), 129.52 (d, o-C_{Bz}), 128.31 (d, m-C_{Bz}), 72.23 (d, C-8), 68.12 (d, C-25), 56.32 (d), 51.58 (d), 41.89 (s, C-13) 39.89 (t), 39.72 (t), 35.61 (t), 35.32 (d), 30.53 (t) 27.07 (t), 2357 (q, C-27), 22.62 (t), 22.12 (i), 18.54 (q, C-21), 18.00 (t), 13.51 (q, C-18); MS (EI) m/z 372 (15, M), 354 (3, M⁺-H₂O), 327 (1, M⁺-CH₅O), 285 (2, M⁺-C₅H₁₁O), 267 (5, M⁺-PhCO), 250 (73, M⁺-PhCOOH), 232 (38), 217 (10), 163 (40), 135 (79), 105 (100); exact mass calculated for C₂₄H₃₆O₃ (M⁺) 372.2664, found 372.2671.

### Preparation of (8S,20R)-de-A,B-8-benzoyloxy-20-[(4S)-tert-butyldimethylsiyloxy-pentyl]pregnane (10c)

tert-Butyldimethylsilyl trifluoromethanesulfonate (37 L, 42 mg, 0.16 mmol) was added to a solution of the alcohol 9c (32 mg, 0.09 mmol) and 2,6-lutidine (37 L, 34 mg, 90.32 mmol) in anhydrous methylene chloride (3 mL) at −20° C. The mixture was stirred under argon at 0° C. for 1 h. The reaction was quenched with water and extracted with methylene chloride. The combined organic phases were washed with brine, dried (Na₂SO₄) and concentrated under reduced pressure. The residue was chromatographed on silica gel with hexane and hexane/ethyl acetate (97:3) to give the product 10c (42 mg, 96%):

[α]_{D}+58.1 (c 1.6 CHCl₃); ¹H NMR (400 MHz, CDCl₃+TMS) δ 8.06 (2H, m, o-H_{Bz}), 7.55 (1H, m, p-H_{Bz}), 7.44 (2H, m, m-H_{Bz}), 5.41 (1H, s, 8α-H), 3.77 (1H, m, 25-H), 2.04 (2H, m), 1.84 (2H, m), 1.12 (3H, d, J=6.0 Hz, 27-H₃), 1.05 (3H, s, 18-H₃), 0.93 (3H, d, J=6.5 Hz, 21-H₃), 0.89 (9H, s, Si-t-Bu), 0.05 (6H, s, SiMe₂); ¹³C NMR (100 MHz) δ 166.48 (s, C═O), 132.64 (d, p-C_{Bz}), 130.92 (s, i-C_{Bz}), 129.55 (d, o-C_{Bz}), 128.32 (d, m-C_{Bz}), 72.27 (d, C-8), 68.67 (d, C-25), 56.50 (d), 51.62 (d), 41.92 (s, C-13), 40.17 (t), 39.94 (t), 35.75 (t), 35.38 (d), 30.56 (t), 27.10 (t), 25.91 (q, SiCMe₃), 23.89 (q, C-27), 22.65 (t), 22.20 (t), 18.53 (q, C-21), 18.16 (s, SiCMe₃), 18.04 (t), 13.54 (q, C-18), −4.36 (q, SiMe), −4.67 (q, SiMe); MS (EI) m/z 486 (1, M⁺), 471 (1, M⁺-CH₃), 307 (8, M⁺-PhCOOH—C₄H₉), 233 (69, M⁺-PhCOOH-t-BuSiMe₂O), 197 (71), 179 (95), 163 (78), 135 (72), 105 (100); exact mass calculated for C₁₉H₃₅OSi (M⁺-PhCOOH—C₄H₉) 307.2457, found 307.2453.

### Preparation of (8S,20R)-de-A,B-20-[(4S)-tert-butyldimethylsilyloxy-pentyl]pregnan-8-ol (11c)

A solution of sodium hydroxide in ethanol (2.5M, 2 mL) was added to a stirred solution of the benzoate 10c (42 mg, 86 μmol) in anhydrous ethanol (10 mL) and the reaction mixture was refluxed for 18 h. The mixture was cooled to room temperature, neutralized with 5% aq. HCl and extracted with dichloromethane. Combined organic phases were washed with saturated aq. NaHCO₃, dried (Na₂SO₄) and evaporated. The residue was chromatographed on silica gel with hexane/ethyl acetate (95:5) to give the alcohol 11c (24 mg, 73% yield):

[α]_{D}+37.3 (c 1.0, CHCl₃); ¹H NMR (400 MHz, CDCl₃+TMS) δ 4.07 (1H, d, J=1.9 Hz, 8α-H), 3.77 (1H, m, 25-H), 2.00 (1H, m), 1.82 (3H, m), 1.11 (3H, d, J=6.1 Hz, 27-H₃), 0.93 (3H, s, 18-H₃), 0.89 (3H, d, 21-H₃) covered by 0.89 (9H, s, Si-t-Bu), 0.05 (6H, s, SiMe₂), ¹³C NMR (100 MHz) δ 69.44 (d, C-8), 68.69 (d, C-25), 56.72 (d), 52.60 (d) 41.83 (s, C-13), 40.38 (t), 40.21 (t), 35.80 (t), 35.24 (d), 33.57 (t), 27.16 (t), 25.91 (q, SiCMe₃), 23.86 (q, C-27), 22.51 (t), 22.21 (t), 18.48 (q, C-21), 18.16 (s, SiCMe₃), 17.43 (t), 13.5 (q, C-18), −4.38 (q, SiMe), −4.68 (q, SiMe), MS (EI) m/z 382 (2, M⁺), 367 (3, M⁺-CH₃), 325 (9, M⁺-C₄H₉), 307 (4, M⁺-C₄H₉—H₂O), 233 (61), 191 (45), 177 (75), 159 (70), 135 (84), 123 (85), 109 (96), 97 (100); exact mass calculated for C₁₉H₃₇O₂Si (M⁺-C₄H₉) 325.2563, found 325.2575.

### Preparation of (20R)-de-A,B20-[(4S)-tert-butyldimethylsilyloxy-pentyl]pregnan-8-one (12c)

Pyridinium dichromate (118, mg, 315 μmol) was added to a solution of the alcohol 11c (24 mg, 63 μmol) and pyridinium p-toluenesulfonate (3 mg, 12 μmol) in anhydrous methylene chloride (5 mL). The resulting suspension was stirred at room temperature for 3 h. The reaction mixture was filtered through a Waters silica Sep-Pak cartridge (5 g) that was further washed with hexane/ethyl acetate (8:2). After removal of solvents the ketone 12c (18 mg, 75% yield) was obtained as a colorless oil:

[α]_{D}+11.9 (c 0.9, CHCl₃); ¹H NMR (400 MHz, CDCl₃+TMS) δ 3.77 (1H, m, 25-H), 2.44 (1H, dd, J=11.5, 7.6 Hz), 1.11 (3H, d, J=6.1 Hz, 27-H₃), 0.94 (3H, d, J=5.9 Hz 21-H₃), 0.88 (9H, s, Si-t-Bu), 0.63 (3H, s, 18-H₃), 0.04 (6H, s, SiMe₂); ¹³C NMR (100 MHz) δ 212.18 (s), 68.62 (d, C-25), 62.00 (d), 56.73 (d), 49.93 (s, C-13), 40.97 (t), 40.10 (t), 38.98 (t), 35.80 (t), 35.46 (d), 27.51 (t), 25.90 (q, SiCMe₃), 24.07 (t), 23.87 (q, C-27), 22.17 (t), 19.06 (t), 18.65 (q, C-21), 18.16 (s, SiCMe₃), 12.47 (q, C-18), −4.36 (q, SiMe), −4.69 (q, SiMe); MS (EI) m/z no M⁺, 379 (1, M⁺-H), 365 (4, M⁺-CH₃), 323 (48, M⁺-C₄H₉), 281 (34), 250 (39), 231 (56), 207 (41), 189 (32), 159 (62), 125 (70), 75 (100); exact mass calculated for C₁₉H₃₅O₂Si (M⁺-C₄H₉) 323.2406, found 323.2415.

### Preparation of (20R,25S)-2-Methylene-19,26-dinor-1α,25-dihydroxyvitamin D₃ (15c)

To a solution of phosphine oxide 13c (73 mg, 125 μmol) in anhydrous THF (400 μL) at −20° C. was slowly added PhLi (1.8 M in di-n-butylether, 85 μL, 153 μmol) under argon with stirring. The solution turned deep orange. After 30 min the mixture was cooled to −78° C. and a precooled (−78° C.) solution of ketone 12c (18 mg, 47 μmol) in anhydrous THF (200+100 μL) was slowly added. The mixture was stirred under argon at −78° C. for 3 h and at 0° C. for 18 h. Ethyl acetate was added, and the organic phase was washed with brine, dried (Na₂SO₄) and evaporated. The residue was dissolved in hexane and applied on a Waters silica Sep-Pak cartridge (2 g). The cartridge was washed with hexane and hexane/ethyl acetate (99.5:0.5) to give 19-norvitamin derivative 14c (25 mg, 71% yield). Then the Sep-Pak was washed with ethyl acetate to recover diphenylphosphine oxide 13c (43 mg). For analytical purpose a sample of the protected vitamin 14c was further purified by HPLC (9.4×250 mm Zorbax Sil column, 4 mL/min, hexane/2-propanol (99.9:0.1) solvent system, Rₜ=3.77 min):

UV (in hexane) λₘₐₓ 263.1, 253.2, 244.3 nm; ¹H NMR (400 MHz, CDCl₃) δ 6.22 and 5.84 (each 1H, each d, J=11.2 Hz, 6- and 7-H), 4.97 and 4.92 (each 1H, each s, ═CH₂), 4.43 (2H, m, 1β- and 3α-H), 3.78 (1H, m, 25-H), 2.82 (1H, dm, J=11.8 Hz, 9β-H), 2.52 (1H, dd, J=13.1, 5.9 Hz, 10α-H), 2.47 (1H, dd, J=12.6, 4.3 Hz, 4α-H), 2.33 (1H, dd, J=13.1, 2.3 Hz, 10β-H), 2.18 (1H, dd, J=12.6, 8.7 Hz, 4β-H), 2.00 (2H, m), 1.12 (3H, d, J=6.0 Hz; 27-H₃), 0.92 (3H, d, J=6.4 Hz, 21-H₃), 0.898 (9H, s, Si-t-Bu), 0.894 (9H, s, Si-t-Bu), 0.867 (9H, s, Si-t-Bu), 0.546 (3H, s, 18-H₃), 0.082 (3H, s, SiMe), 0.068 (3H, s, SiMe), 0.054 (9H, s, 3×SiMe), 0.028 (3H, s, SiMe); ¹³C NMR (100 MHz) δ 152.99 (s, C-2), 141.27 (s, C-8), 132.69 (s, C-55) 122.43 (d, C-6), 116.09 (d, C-7), 106.25 (t, ═CH₂), 72.54 and 71.63 (each d, C-1 and C-3), 68.73 (d, C-25), 56.63 (d), 56.29 (d), 47.61 (t), 45.67 (s, C-13), 40.61 (t), 40.24 (t), 38.55 (t), 36.13 (d), 35.98 (t), 28.76 (t), 27.73 (t), 25.93 (q, SiCMe₃), 25.85 (q, SiCMe₃), 25.78 (q, SiCMe₃), 23.89 (q, C-27), 23.45 (t), 22.33 (t), 22.22 (t), 18.77 (q, C-21), 18.25 (s, SiCMe₃), 18.17 (s, 2×SiCMe₃), 12.06 (q, C-18), −4.37 (q, SiMe), −4.66 (q, SiMe), −4.86 (q, 3×SiMe), −5.09 (q, SiMe); exact mass calculated for C₄₄H₈₄O₃Si₃Na (MNa⁺) 767.5626, found 767.5646.

The protected vitamin 14c (25 mg, 34 μmol) was dissolved in THF (2 mL) and acetonitrile (2 mL). A solution of aq. 48% HF in acetonitrile (1:9 ratio, 2 mL) was added at 0° C. and the resulting mixture was stirred at room temperature for 8 h. Saturated aq. NaHCO₃ solution was added aid the reaction mixture was extracted with ethyl acetate. The combined organic phases were washed with brine, dried (Na₂SO₄) and concentrated under reduced pressure. The residue was diluted with 2 mL of hexane/ethyl acetate (8:2) and applied on a Waters silica Sep-Pak cartridge (2 g). An elution with hexane/ethyl acetate (8:2) and later with ethyl acetate gave the crude product 15c (15 mg). The vitamin 15c was further purified by straight phase HPLC [9.4×250 mm Zorbax Sil column, 4 mL/min, hexane/2-propanol (85:15) solvent system, Rₜ=9.31 min.] and by reverse phase HPLC [9.4×250 mm Zorbax Eclipse XDB-C18 column, 3 mL/min, methanol/water (85:15) solvent system, Rₜ=10.16 min.] to give a colorless oil (12.6 mg, 92% yield):

UV (in EtOH) λₘₐₓ 262.1, 252.6, 244.1 nm; ¹H NMR (600 MHz, CDCl₃) δ 6.35 and 5.88, (1H and 1H, each d, J=11.2 Hz, 6- and 7-H), 5.10 and 5.08 (each 1H, each s, ═CH₂), 4.47 (2H, m, 1β- and 3α-H), 3.80 (1H, m, 25-H), 2.83 (1H, dd, J=13.3, 4.5 Hz, 10β-H), 2.81 (1H, br d, J=13.2 Hz, 9β-H), 2.56 (1H, dd, J=13.4, 3.7 Hz, 4α-H), 2.32 (1H, dd, J=13.4, 6.1 Hz, 4β-H), 2.29 (1H, dd, J=13.3, 8.3 Hz, 10α-H), 1.19 (3H, d, J=6.2 Hz, 27-H₃), 0.193 (3H, d, J=6.4 Hz, 21-H₃), 0.546 (3H, s, 18-H₃); ¹³C NMR (100 MHz) δ 151.97 (s, C-2), 143.39 (s, C-8), 130.42 (s, C-5), 124.20 (d, C-6), 115.28 (d, C-7), 107.70 (t, ═CH₂), 71.79 and 70.62 (each d, C-1 and C-3), 68.18 (d, C-25), 56.43 (d), 56.30 (d), 45.76 (t), 45.76 (s, C-13), 40.42 (t), 39.75 (t), 38.13 (t), 36.02 (d), 35.80 (t), 28.94 (t), 27.63, (t), 23.54 (q, C-27), 23.48 (t), 122.26 (t), 22.17 (t), 18.78 (q, C-21), 12.06 (q, C-18); MS (EI) m/z 402 (35, M⁺), 384 (2, M⁺-H₂O), 369 (2, M⁺-H₂O—CH₃), 329 (65, M⁺-C₄H₉O), 287 (13, M⁺-C₇H₁₅O), 257 (100), 229 (17), 159 (31), 145 (46), 115 (65), 91 (96); exact mass calculated for C₂₆H₄₂O₃ (M⁺) 402.3134, found 402.3129.

### Preparation of (20R,25S-2α-methyl-19,26-dinor-1α,25-dihydroxyvitamin D₃ (16) and (20R,25S)-2β-methyl-19,26-dinor-1α,25-dihydroxyvitamin D₃ (17)

Tris(triphenylphosphine)rhodium (1) chloride (10 mg, 10.8 μmol) was added to dry benzene (5 mL) presaturated with hydrogen (15 min). The mixture was stirred at room temperature until a homogeneous solution was formed (ca. 25 min). A solution of vitamin 15c (2.8 mg, 7.0 μmol) in dry benzene (3 mL) was then added and the reaction was allowed to proceed under a continuous stream of hydrogen for 4 h. Benzene was removed under vacuum, the residue was redissolved in hexane/ethyl acetate (1:1) and applied on a Waters silica Sep-Pak cartridge (2 g). A mixture of 2-methyl vitamins was eluted with the same solvent system. The compounds were further purified by HPLC (9.4×250 mm Zobax RX-Sil column, 4 mL/min) using hexane/2-propanol (85:15) solvent system. The mixture of 2-methyl-19-norvitamins 16c and 17c gave a single peak at Rₜ=7.5 min. Separation of both epimers was achieved by reversed-phase HPLC (9.4×250 mm Zorbax RX C18 column, 3 mL/min) using methanol/water (85:15) solvent system. 2β-Methyl vitamin 17c (754 μg, 27% yield) was collected at Rₜ=9.6 min. and its 2α-epimer 16c (820 μg, 29% yield) at Rₜ=10.9 min:

2α-Methyl analog 16c: UV (in EtOH) λₘₐₓ 260.0, 251.0, 243.5 nm; ¹H NMR (500 MHz, CDCl₃) δ 6.37 and 5.82 (1H and 1H, each d, J=11.3 Hz, 6- and 7-H), 3.96 (1H, m, 1β-H), 3.80 (3H, m, 25-H), 3.61 (1H, m, 3α-H), 2.80 (2H, br m, 9β- and 10α-H), 2.60 (1H, dd, J=12.8, 4.5 Hz, 4α-H), 2.23 (1H, br d, J=13.3 Hz, 10β-H), 2.13 (1H, ˜t, J˜11.1 Hz, 4β-H), 1.194 (3H, d, J=6.2 Hz, 27-H₃), 1.133 (3H, d, J=6.9 Hz, 2α-CH₃), 0.930 (3H, d, J=6.5 Hz, 21-H₃), 0.533 (3H, s, 18-H₃); MS (EI) m/z 404 (98, M⁺), 386 (31, M⁺-H₂O), 368 (12, M⁺-2H₂O), 353 (8, M⁺-2H₂O—CH₃), 317 (22, M⁺-C₅H₁₀OH), 289 (73, M⁺-C₇H₁₄OH), 271 (53, M⁺-C₇H₁₄OH—H₂O), 253 (51), 231 (24), 194 (31), 177 (41), 161 (52), 147 (81), 135 (100); exact mass calculated for C₂₆H₄₄O₃ (M⁺) 404.3290, found 404.3280.

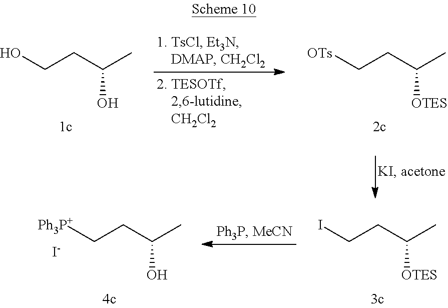

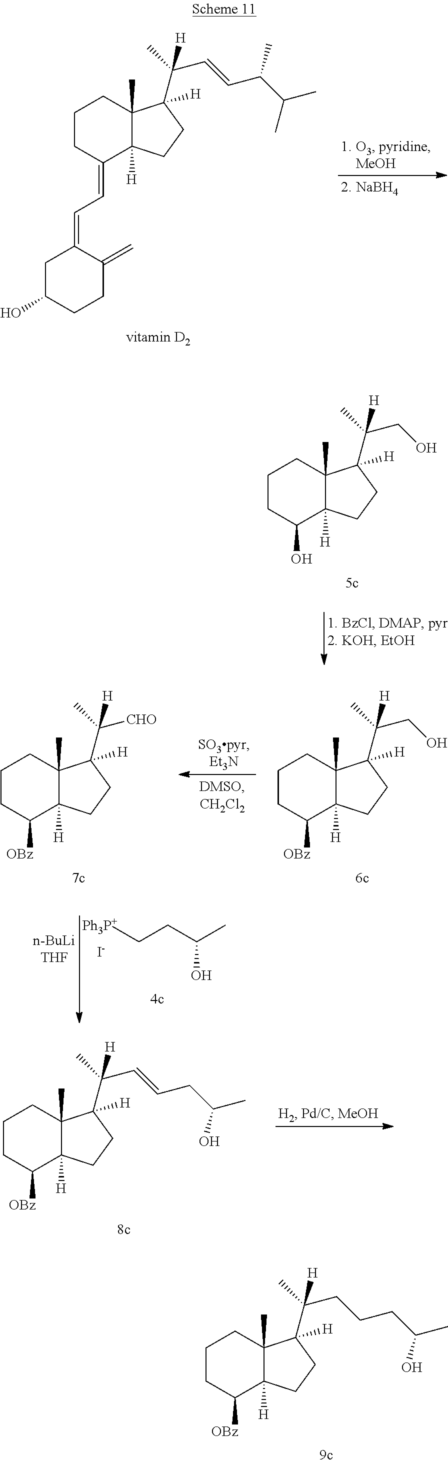

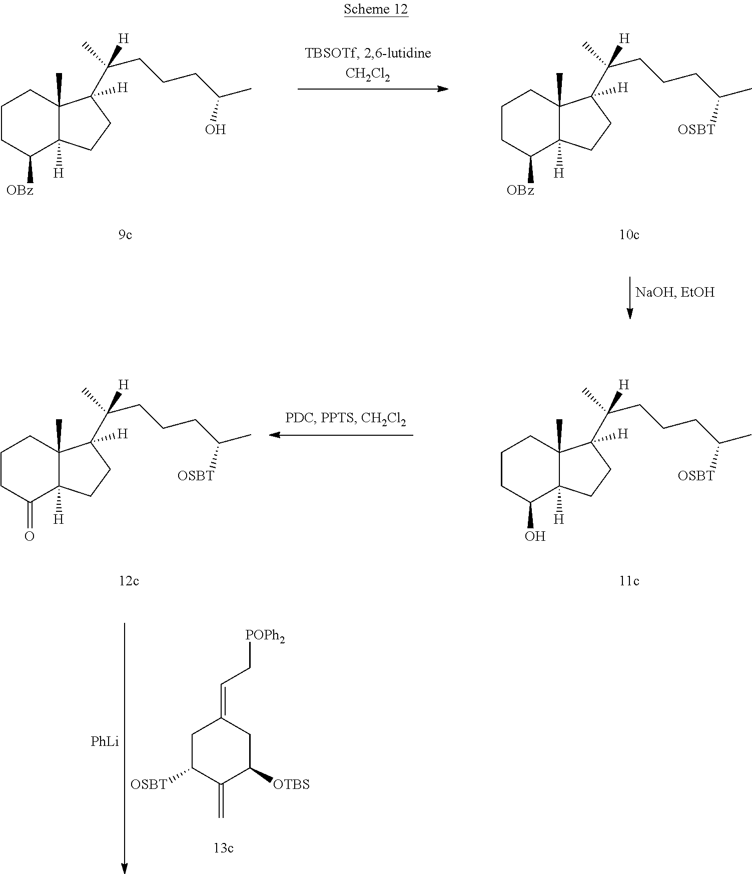

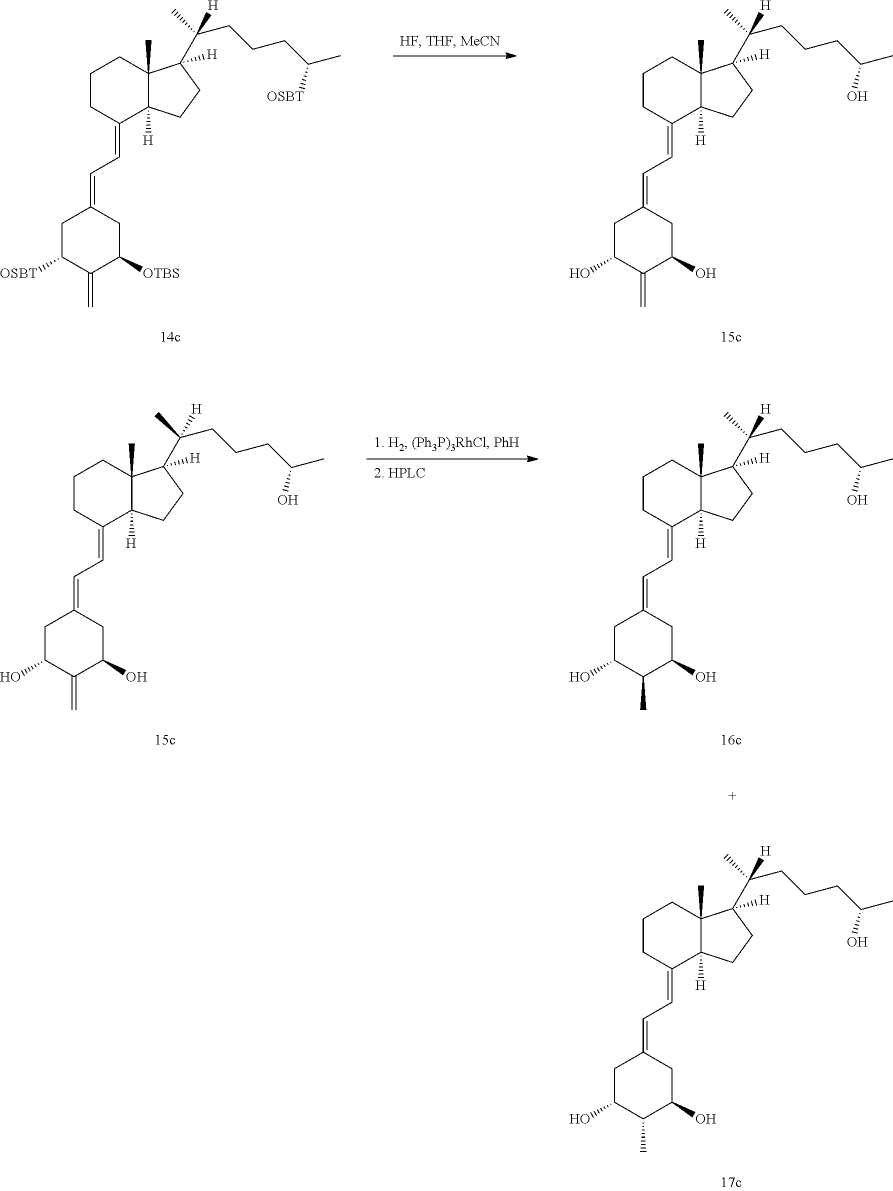

### Experimental Methods

### Vitamin D Receptor Binding

Test Material

Protein Source

Full-length recombinant rat receptor was expressed in E. coli BL21 (DE3) Codon Plus RIL cells and purified to homogeneity using two different column chromatography systems. The first system was a nickel affinity resin that utilizes the C-terminal histidine tag on this protein. The protein that was eluted from this resin was further purified using ion exchange chromatography (S-Sepharose Fast Flow). Aliquots of the purified protein were quick frozen in liquid nitrogen and stored at −80° C. until use. For use in binding assays, the protein was diluted in TEDK₅₀ (50 mM Tris, 1.5 mM EDTA, pH 7.4, 5 mM DTT, 150 mM KCl) with 0.1% Chaps detergent. The receptor protein and ligand concentration was optimized such that no more than 20% of the added radiolabeled ligand as bound to the receptor.

Study Drugs

Unlabeled ligands were dissolved in ethanol and the concentrations determined using UV spectrophotometry (1,25(OH)₂D3: molar extinction coefficient=18,200 and λₘₐₓ=265 nm; Analogs: molar extinction coefficient=42,000 and λₘₐₓ=252 nm) Radiolabeled ligand (3H-1,25(OH)₂D₃, ˜159 Ci/mmole) was added in ethanol at a final concentration of 1 nM.

Assay Conditions

Radiolabeled and unlabeled ligands were added to 100 mcl of the diluted protein at a final ethanol concentration of ≦10%; mixed and incubated overnight on ice to reach binding equilibrium. The following day, 100 mcl of hydroxylapaptite slurry (50%) was added to each tube and mixed at 10-minute intervals for 30 minutes. The hydroxylapaptite was collected by centrifugation and then washed three times with Tris-EDTA buffer (50 mM Tris, 1.5 mM EDTA, pH 7.4) containing 0.5% Titron X-100. After the final wash, the pellets were transferred to scintillation vials containing 4 ml of Biosafe II scintillation cocktail, mixed and placed in a scintillation counter. Total binding was determined from the tubes containing only radiolabeled ligand.

HL-60 Differentiation

Test Material

Study Drugs

The study drugs were dissolved in ethanol and the concentrations determined using UV spectrophotometry. Serial dilutions were prepared so that a range of drug concentrations could be tested without changing the final concentration of ethanol (≦0.2%) present in the cell cultures.

Cells

Human promyelocytic leukemia (HL60) cells were grown in RPMI-1640 medium containing 10% fetal bovine serus. The cells were incubated at 37° C. in the presence of 5% CO₂.

Assay Conditions

HL60 cells were plated at 1.2×10⁵ cells/ml. Eighteen hours after plating, cells in duplicate were treated with drug. Four days later, the cells were harvested and a nitro blue tetrazolium reduction assay was performed (Collins et al., 1979; J. Ex. Med. 149:969-974). The percentage of differentiated cells was determined by counting a total of 200 cells and recording the number that contained intracellular black-blue formazan deposits Verification of differentiation to monocytic cells was determined by measuring phagocytic activity (data not shown).

In Vitro Transcription Assay

Transcription activity was measured in ROS 17/2.8 (bone) cells that were stably transfected with a 24-hydroxylase (24Ohase) gene promoter upstream of a luciferase reporter gene (Arbour et al., 1998). Cells were given a range of doses. Sixteen hours after dosing the cells were harvested and luciferase activities were measured using a luminometer.

RLU=relative luciferase units.

Intestinal Calcium Transport and Bone Calcium Mobilization

Male, weanling Sprague-Dawley rats were placed on Diet 11 (0.47% Ca) diet +AEK for one week followed by Diet 11 (0.02% Ca) +AEK for 3 weeks. The rats were then switched to a diet containing 0.47% Ca for one week followed by two, weeks on a diet containing 0.02% Ca. Dose administration began during the last week on 0.02% calcium diet. Four consecutive ip doses were given approximately 24 hours apart. Twenty-four hours after the last dose, blood was collected from the severed neck and the concentration of serum calcium determined as a measure of bone calcium mobilization. The first 10 cm of the intestine was also collected for intestinal calcium transport analysis using the everted gut sac method.

### Example 5

### Biological Activity OF LR-2

With regard to the 2α-methyl analog LR-2, the introduction of a methyl group in an alpha orientation to the 2-position and the removal of a methyl group at the 26 position in the side chain of (20S,25R)-19-nor-1α,25-dihydroxyvitamin D₃ had little or no effect on binding to the full length recombinant rat vitamin D receptor, as compared to 1α,25-dihydroxyvitamin D₃. The compound LR-2 bound equally well to the receptor as compared to the standard 1,25-(OH)₂D₃ (FIG. 1). It might be expected from these results that compound LR-2 would have equivalent biological activity. Surprisingly, however, compound LR-2 is a highly selective analog with unique biological activity.

FIG. 4 demonstrates that LR-2 is more than 30 times less potent than 1,25(OH)₂D₃ in bone, and thus has very little bone calcium mobilization activity, as compared to 1,25(OH)₂D₃. FIG. 5 demonstrates that LR-2 is approximately one log less potent than 1,25(OH)₂D₃ in the intestine. Thus, LR-2 may be characterized as having little, if any, calcemic activity.

FIG. 2 illustrates the LR-2 is 10 times more potent than 1,25(OH)₂D₃ on HL-60 differentiation, making it an excellent candidate for the treatment of psoriasis and cancer, especially against leukemia, colon cancer, breast cancer, skin cancer and prostate cancer. In addition, due to its relatively high cell differentiation activity, this compound provides a therapeutic agent for the treatment of various skin conditions including wrinkles, lack of adequate dermal hydration, i.e. dry skin, lack of adequate skin firmness, i.e. slack skin, and insufficient sebum secretion. Use of this compound thus not only results in moisturizing of skin but also improves the barrier function of skin.

FIG. 3 illustrates that the compound LR-2 also has 10 times more transcriptional activity than 1α,25-dihydroxyvitamin D₃ in bone cells. This result, together with the cell differentiation activity of FIG. 2, suggests that LR-2 will be very effective in psoriasis because it has direct cellular activity in causing cell differentiation and in suppressing cell growth. These data also indicate that LR-2 may have significant activity as an anti-cancer agent, especially against leukemia, colon cancer, breast cancer, skin cancer and prostate cancer.

The strong activity of LR-2 on HL-60 differentiation suggests it will be active in suppressing growth of parathyroid glands and in the suppression of the preproparathyroid gene.

### Interpretation of LR-2Data

VDR Binding, IL-60 Cell Differentiation, and Transcription Activity.

LR-2 (Kᵢ-8×10⁻¹¹M) is equivalent to the natural hormone 1α,25-dihydroxyvitamin D₃ (Kᵢ-1×10⁻¹⁰M) in its ability to compete with [³H]-1,25(OH)₂D₃ for binding to the full length recombinant rat vitamin D receptor (FIG. 1). LR-2 is 10 times more potent (EC₅₀=1×10⁻¹⁰M) in its ability (efficacy or potency) to promote HL-60 cell differentiation as compared to 1α,25-dihydroxyvitamin D₃ (EC₅₀=2×10⁻¹⁰M) (See FIG. 3).

These results suggest that LR-2 will be very effective in psoriasis because it has direct cellular activity in causing cell differentiation and in suppressing cell growth. These data also indicate that LR-2 will have significant activity as an anti-cancer agent, especially against leukemia, colon cancer, breast cancer, skin cancer and prostate, cancer, as well as against skin conditions such as dry skin (lack of dermal hydration), undue skin slackness (insufficient skin firmness), insufficient sebum secretion and wrinkles.

LR-2 would also be expected to be very active in suppressing secondary hyperparathyroidism.

Calcium Mobilization from Bone in Vitamin D-Deficient Animals.

Using vitamin D-deficient rats on a low calcium diet (0.02%), the activities of LR-2 and 1,25(OH)₂D₃ in intestine and bone were tested. As expected, the native hormone (1,25(OH)₂D) increased serum calcium levels at all dosages (FIG. 4). FIG. 4, however, also shows that LR-2 has little, if any, activity in mobilizing, calcium from bone. Administration of LR-2 at 7,020 pmol/day for 4 consecutive days did not result in mobilization of bone calcium, and increasing the amount of LR-2 to 21,060 pmol/day was also without any substantial effect.

Intestinal Calcium Transport Activity.

FIG. 5 demonstrates that 1,25(O)₂D₃ has significant activity in stimulating calcium transport in the gut, as expected. FIG. 5, however, also shows that LR-2 is about 10 times (one log) less potent than 1,25(OH)₂D₃ in stimulating calcium transport in the gut. Administration of LR-2 at 7,020 pmol/day for 4 consecutive days resulted in stimulating some intestinal calcium transport activity, but still not to the same extent as 1,25(OH)₂D₃, at only 780 pmol/day.

These results illustrate that LR-2 is an excellent candidate for numerous human therapies as described herein, and that it may be particularly useful in a number of circumstances such as suppression of secondary hyperparathyroidism of renal osteodystrophy, autoimmune diseases, cancer, and psoriasis. LR-2 is an excellent candidate for treating psoriasis because: (1) it has significant transcription activity and cellular differentiation activity; (2) it is devoid of hypercalcemic liability unlike 1,25(OH)₂D₃; and (3) it is easily synthesized. Also, since LR-2 has significant binding activity to the vitamin D receptor, but has little ability to raise blood serum calcium, it may also be particularly useful for the treatment of secondary hyperparathyroidism of renal osteodystrophy.

These data also indicate that the compound LR-2 of the invention may be especially suited for treatment and prophylaxis of human disorders which are characterized by an imbalance in the immune system, e.g. in autoimmune diseases, including multiple sclerosis, lupus, diabetes mellitus, host versus graft rejection, and rejection of organ transplants; and additionally for the treatment of inflammatory diseases, such as rheumatoid arthritis, asthma, and inflammatory bowel diseases such as celiac disease, ulcerative colitis and Crohn's disease. Acne, alopecia and hypertension are other conditions which may be treated with the compound LR-2 of the invention.

The compound LR-2 is also useful, in preventing or treating obesity, inhibiting adipocyte differentiations, inhibiting SCD-1 gene transcription, and/or reducing body fat in animal subjects. Therefore; in some embodiments, a method of preventing or treating obesity, inhibiting adipocyte differentiations, inhibiting SCD-1 gene transaction, and/or reducing body fat in an animal subject includes administering to the animal subject, an effective amount of LR-2 or a pharmaceutical composition that includes LR-2. Administration of the compound or the pharmaceutical compositions to the subject inhibits adipocyte differentiation, inhibits gene transcription, and/or reduces body fat in the animal subject.

### Example 6

### Biological Activity of FD-1

With regard to the 2β-methyl analog FD-1, the introduction of a methyl group in a beta orientation to the 2-position and the removal of a methyl group at the 26 position in the side chain of (20S,25R)-19-nor-1α,25-dihydroxyvitamin D₃ reduced its ability to bind to the full length recombinant rat vitamin D receptor, as compared to 1α,25-dihydroxyvitamin D₃. The compound FD-1 exhibits 4 times lower activity in binding to the receptor as compared to the standard 1,25-(OH)₂D₃ (FIG. 6). It might be expected from these results that compound FD-1 would not have any desirable biological activity. Surprisingly, however, compound FD-1 is a highly selective analog with unique biological activity.

FIG. 9 demonstrates that FD-1 is more than 40 times less potent than 1,25(OH)₂D₃ in bone; and thus; has very little bone calcium mobilization: activity, as compared to 1,25(OH)₂D3. FIG. 10 demonstrates that FD-1 is approximately 40 times less potent that 1,25(OH)₂D₃ in the intestine. Thus, FD-1 may be characterized as having-little, if any, calcemic activity.

FIG. 7 illustrates that FD-1 is only 3 times less potent than 1,25(OH)₂D₃ on HL-60 differentiation, making it a candidate for the treatment of psoriasis and cancer, especially against leukemia, colon cancer, breast cancer, skin cancer and prostate cancer. In addition, due to its relatively high cell differentiation activity, this compound provides a therapeutic agent for the treatment of various skin conditions including wrinkles, lack of adequate dermal hydration, i.e. dry skin, lack of adequate skin, firmness, i.e. slack skin, and insufficient sebum secretion. Use of this compound thus not only results in moisturizing of skin but also improves the barrier function of skin.

FIG. 8 illustrates that the compound FD-1 has about 10 times less transcriptional activity than 1α,25-dihydroxyvitamin D₃ in bone cells. This result, together with the cell differentiation activity of FIG. 7, suggests that FD-1 will be very effective in psoriasis because it has direct cellular activity in causing cell differentiation and in suppressing cell growth. These data also indicate that FD-1 may have significant activity as an anti-cancer agent, especially against leukemia, colon cancer, breast cancer skin cancer and prostate cancer.

The strong activity of FD-1 on HL-60 differentiation suggests it will be active in suppressing growth of parathyroid glands and in the suppression of the preproparathyroid gene.

### Interpretation of FD-1 Data

VDR Binding HL-60 Cell Differentiation, and Transcription Activity.

FD-1 (Kᵢ=4×10⁻¹⁰ M) is nearly equivalent (i.e. only about 4 times less active) to the natural hormone 1α,25-dihydroxyvitamin D₃ (Kᵢ=1×10⁻¹⁰M) in its ability to compete with [³H]-1,25(OH)₂D₃ for binding to the full-length recombinant rat vitamin D, receptor (FIG. 6). Also, FD-1 is only about 3 times less potent (EC₅₀=6×10⁻⁹M) in its ability (efficacy or potency) to promote HL-60 differentiation as compared to 1α,25-dihydroxyvitamin D₃ (EC₅₀=2×10⁻⁹M) (See FIG. 7). Also, compound FD-1 (EC₅₀=1×10⁻⁹M) has only about 1 log less transcriptional, activity in bone cells as compared to 1α,25-dihydroxyvitamin D₃ (EC₅₀=2×10⁻¹⁰M) (See FIG. 8).

These results suggest that FD-1 will be very effective in psoriasis because it has direct cellular activity in causing cell differentiation and in suppressing cell growth. These data also indicate that FD-1 will have significant activity as an anti-cancer agent, especially against, leukemia, colon cancer, breast cancer, skin cancer and prostate cancer, as well as against skin conditions such as dry skin (lack of dermal hydration), undue skin slackness (insufficient skin firmness), insufficient sebum secretion and wrinkles.

FD-1 would also be expected to be very active in suppressing secondary hyperparathyroidism.

Calcium Mobilization from Bone in Vitamin D-Deficient Animals.

Using, vitamin D-deficient rats on a low calcium diet (0.02%), the activities of FD-1 and 1,25(OH)₂D₃ in intestine and bone were tested. As expected, the native hormone (1,25(OH)₂D₃) increased serum calcium levels at all dosages (FIG. 9), FIG. 9, however, also shows that FD-1 is more than 40 times less potent than 1,25(OH)₂D₃; and thus FD-1 has little, if any, activity in mobilizing calcium from bone. Administration of FD-1 at 21,060 pmol/day for 4 consecutive days did not result in mobilization of bone calcium.

Intestinal Calcium Transport Activity.

FIG. 10 demonstrates that 1,25(OH)₂D₃ has significant activity in stimulating calcium transport in the gut, as expected. FIG. 10, however, also shows that FD-1 has some, but very little, intestinal calcium transport activity, as compared to 1,25(OH)₂D₃. Administration of FD-1 at 21,060 pmol/day for 4 consecutive days resulted in stimulating some intestinal calcium transport, but hot to the same extent as 1,25(OH)₂D₃ at only 780 pmol/day.

These results illustrate that FD-1 is an excellent candidate for numerous human therapies as described herein, and that it may be particularly useful in a number of circumstances such as suppression of secondary hyperparathyroidism of renal osteodystrophy, autoimmune diseases, cancer, and psoriasis. FD-1 is an excellent candidate for treating psoriasis because: (1) it has significant transcription activity and cellular differentiation activity; (2) it is devoid of hypercalcemic liability unlike 1,25(OH)₂D₃; and (3) it is easily synthesized. Also, since FD-1 has significant binding activity to the vitamin D receptor, but has little ability to raise blood serum calcium, it may also be particularly useful for the treatment of secondary hyperparathyroidism of renal osteodystrophy.

These data also indicate that the compound FD-1 of the invention may be especially suited for treatment and prophylaxis of human disorders which are characterized by an imbalance in the immune system, e.g. in autoimmune diseases, including multiple sclerosis, lupus, diabetes mellitus, host versus graft rejection, and rejection of organ transplants and additionally for the treatment of inflammatory diseases such as rheumatoid arthritis, asthma, and inflammatory bowel diseases such as celiac disease, ulcerative colitis and Crohn's disease. Acne, alopecia and hypertension are other conditions which may be treated with the compound FD-1 of the invention.

The compound FD-1 is also useful in preventing or treating obesity, inhibiting adipocyte differentiations, inhibiting SCD-1 gene transcription, and/or reducing body fat in animal subjects. Therefore in some embodiments, a method of preventing or treating obesity, inhibiting adipocyte differentiations, inhibiting SCD-1 gene transcription, and/or reducing body fat in an animal subject includes administering to the animal subject, an effective amount of FD-1 or a pharmaceutical composition that includes FD-1. Administration of the compound or the pharmaceutical compositions to the subject inhibits adipocyte differentiation, inhibits gene transcription, and/or reduces body fat in the animal subject.

### Example 7

### Biological Activity of MY-2

With regard to the 2α-methyl analog MY-2, the introduction of a methyl group in an alpha orientation to the 2-position and the removal of a methyl group at the 26 position in the side chain of (20R,25R)-19-nor-1α,25-dihydroxyvitamin D₃ had little or no effect on binding to the full length recombinant rat vitamin D receptor, as compared to 1α,25-dihydroxyvitamin D₃. The compound MY-2 bound equally well to the receptor as compared to the standard 1,25-(OH)₂D₃ (FIG. 11). It might be expected from these results that compound MY-2 would have equivalent biological activity. Surprisingly, however, compound MY-2 is a highly selective analog with unique biological activity.

FIG. 14 demonstrates that MY-2 is more than 50 times less potent than 1,25(OH)₂D₃ in bone, and thus has very little bone calcium mobilization activity, as compared to 1,25(OH)₂D₃. FIG. 15 demonstrates that MY-2 is approximately 50 times less potent than 1,25(OH)₂D₃ in the intestine. Thus, MY-2 may be characterized as having little, if any, calcemic activity.

FIG. 12 illustrates that MY-2 is as potent as 1,25(OH)₂D₃ on HL-60 differentiation, making it an excellent candidate for the treatment of psoriasis and cancer, especially against leukemia, colon cancer, breast cancer, skin cancer and prostate cancer. In addition, due to its relatively high cell differentiation activity, this compound provides a therapeutic agent for the treatment of various skin conditions including wrinkles, lack of adequate dermal hydration, i.e. dry skin, lack of adequate skin firmness, i.e. slack skin, and insufficient sebum secretion. Use of this compound thus not only results in moisturizing of skin but also improves the barrier function of skin.

FIG. 13 illustrates that the compound MY-2 has transcriptional activity equivalent to 1α,25-dihydroxyvitamin D₃ in bone cells. This result, together with the cell differentiation activity of FIG. 12, suggests that MY-2 will be very effective in psoriasis because it has direct cellular activity in causing cell differentiation and in suppressing cell growth. These data also indicate that MY-2 may have significant activity as an anti-cancer agent, especially against leukemia, colon cancer, breast cancer, skin cancer and prostate cancer.

The strong activity of MY-2 on HL-60 differentiation suggests it will be active in suppressing growth of parathyroid glands and in the suppression of the preproparathyroid gene.

### Interpretation of MY-2 Data

VDR Binding, HL-60 Cell Differentiation, and Transcription Activity.

MY-2 (Kᵢ=2×10⁻¹⁰M) is, equivalent to the natural hormone 1α,25-dihydroxyvitamin D₃ (Kᵢ=1×10⁻¹⁰M), in its ability to compete with [³H]— 1,25(OH)₂D₃ for binding to the full-length recombinant vitamin D receptor (FIG. 11). There is also little difference between MY-2 (EC₅₀=2×10⁻⁹M) in its ability (efficacy or potency) to promote HL-60 differentiation as compared to 1α,25-dihydroxyvitamin D₃ (EC₅₀=2×10⁻⁹M) (See FIG. 12). Also, compound MY-2 (EC₅₀=4×10⁻¹⁰M) has about the same transcriptional activity in bone cells as compared to 1α,25-dihydroxyvitamin D₃ (EC₅₀=2×10⁻¹⁰ M) (See FIG. 13).

These results suggest that MY-2 will be very effective in psoriasis because it has direct cellular activity in causing cell differentiation and in suppressing cell growth. These data also indicate that MY-2 will have significant activity as an anti-cancer agent, especially against leukemia, colon cancer, breast cancer, skin cancer and prostate cancer, as well as against skin conditions such as dry skin (lack of dermal hydration), undue skin slackness (insufficient skin firmness), insufficient sebum secretion and wrinkles.

MY-2 would also be expected to be very active in suppressing secondary hyperparathyroidism.

Calcium Mobilization from Bone in Vitamin D-Deficient Animals.

Using vitamin D-deficient rats on a low calcium diet (0.02%), the activities of MY-2 and 1,25(OH)₂D₃ in intestine and bone were tested. As expected, the native hormone (1,25(OH)₂D₃) increased serum calcium levels-at-all dosages (FIG. 14). FIG. 14, however, also shows that MY-2 has little, if any, activity in mobilizing calcium from bone. Administration of MY-2 at 7020 pmol/day for 4 consecutive days did not result in mobilization of bone calcium, and increasing the amount of MY-2 to 35,100 pmol/day was also without any substantial effect.

Intestinal Calcium Transport Activity.

FIG. 15 demonstrates that 1,25(OH)₂D₃ has significant activity in stimulating calcium transport in the gut, as expected. FIG. 15, however, also demonstrates that MY-2 is about 50 times less potent than 1,25(OH)₂D₃ in stimulating calcium transport in the gut. Administration of MY-2 at 7020 pmol/day, a dose that is 9 times greater than the 780 pmol/day dose tested for 1,25(OH)₂D₃, resulted in stimulating some intestinal calcium transport activity, but still not to the same extent as 1,25(OH)₂D₃ at 780 pmol/day.

These results illustrate that MY-2 is an excellent candidate for numerous human therapies as described herein, and that it may be particularly useful in a number of circumstances such as suppression of secondary hyperparathyroidism of renal osteodystrophy, autoimmune diseases, cancer, and psoriasis. MY-2 is an excellent candidate for treating psoriasis because: (1) it has significant transcription activity and cellular differentiation activity; (2) it is devoid of hypercalcemic liability unlike 1,25(OH)₂D₃; and (3) it is easily synthesized. Also, since MY-2 has significant binding activity to the vitamin D receptor, but has little ability to raise blood serum calcium, it may also be particularly useful for the treatment of secondary hyperparathyroidism of renal osteodystrophy.

These data also indicate that the compound MY-2 of the invention may be especially suited for treatment and prophylaxis of human disorders which are characterized by an imbalance in the immune system, e.g. in autoimmune diseases, including multiple sclerosis, lupus, diabetes mellitus, host versus graft rejection, and rejection of organ transplants; and additionally for the treatment of inflammatory diseases such, as rheumatoid arthritis, asthma, and inflammatory bowel diseases such as celiac disease, ulcerative colitis and Crohn's disease. Acne, alopecia and hypertension are other conditions which may be treated with the compound MY-2 of the invention.

The compound MY-2 is also useful in preventing or treating obesity, inhibiting adipocyte differentiations, inhibiting SCD-1 gene transcription, and/or reducing body fat in animal subjects. Therefore in some embodiments, a method of preventing or treating obesity, inhibiting adipocyte differentiations, inhibiting SCD-1 gene transcription, and/or reducing body fat in an animal subject includes administering to the animal subject, an effective amount of MY-2 or a pharmaceutical composition that includes. MY-2. Administration of the compound or the pharmaceutical compositions to the subject inhibits adipocyte differentiation, inhibits gene transcription, and/or reduces body fat in the animal subject.

### Example 8

### Biological Activity of DW-1

With regard to the 2β-methyl analog DW-1, the introduction of a methyl group in a beta orientation to the 2-position and the removal of a methyl group at the 26 position in the side chain of (20R,25R)-19-nor-1α,25-dihydroxyvitamin D₃ reduced its ability to bind to the full length recombinant rat vitamin D receptor, as compared to 1α,25-dihydroxyvitamin D₃. The compound DW-1 exhibits 1 log (10 times) lower activity in binding to the receptor as compared to the standard 1,25-(OH)₂D₃ (FIG. 16). It might be expected from these results that compound DW-1 would not have any desirable biological activity. Surprisingly, however, compound DW-1 is a highly selective analog with unique biological activity.

FIG. 19 demonstrates that DW-1 is more than 50 times less potent than 1,25(OH)₂D₃ in bone, and thus has very little bone calcium mobilization activity, as compared to 1,25(OH)₂D₃. FIG. 20 demonstrates that DW-1 is approximately 50 times less potent than 1,25(OH)₂D₃ in stimulating calcium transport in the gut. Thus, DW-1 may be characterized as having little, if any, calcemic activity.

FIG. 17 illustrates that DW-1 is about 30 times less potent than 1,25(OH)₂D₃ on HL-60 differentiation, making it a candidate for the treatment of psoriasis and cancer, especially against leukemia, colon cancer, breast cancer, skin cancer and prostate cancer. In addition, due to its relatively high cell differentiation activity, this compound provides a therapeutic agent for the treatment of various skin conditions including wrinkles, lack of adequate dermal hydration, i.e. dry skin, lack of adequate skin firmness, i.e. slack skin, and insufficient sebum secretion. Use of this compound thus not only results in moisturizing of skin but also improves the barrier function of skin.

FIG. 18 illustrates that the compound DW-1 has about 2 logs (20 times) less transcriptional activity than 1α,25-dihydroxyvitamin D₃ in bone cells. This result, together with the cell differentiation activity of FIG. 17, suggests that DW-1 will be very effective in psoriasis because it has direct cellular activity in causing cell differentiation and in suppressing cell growth. These data also indicate that DW-1 may have significant activity as an anti-cancer agent, especially against leukemia, colon cancer, breast cancer, skin cancer and prostate cancer.

The strong activity of DW-1 on HL-60 differentiation suggests it will be active in suppressing growth of parathyroid glands and in the suppression of the preproparathyroid gene.

### Interpretation of DW-1 Data

VDR Binding, HL-60 Cell Differentiation, and Transcription Activity.

DW-1 (Kᵢ=1×10⁻⁹M) is nearly equivalent (i.e. only about 1 log less active) to the natural hormone 1α,25-dihydroxyvitamin D₃ (Kᵢ=1×10⁻¹⁰M) in its ability to compete with [³H]— 1,25(OH)₂D₃ for binding to the full-length recombinant rat vitamin D receptor (FIG. 16). DW-1 is also only about 30 times less potent (EC₅₀=6×10⁻⁸M) in its ability (efficacy or potency) to promote HL-60 differentiation as compared to 1α,25-dihydroxyvitamin D₃ (EC₅₀=2×10⁻⁹M) (See FIG. 17). Also, compound DW-1 (EC₅₀=2×10⁻⁸M) has significant transcriptional activity in bone cells (i.e. only about 2 logs less potent) as compared to 1α,25-dihydroxyvitamin D₃ (EC₅₀=2×10⁻¹⁰M) (See. FIG. 18).

These results suggest that DW-1 will be very effective in psoriasis because it has direct cellular activity in causing cell differentiation and in suppressing cell growth. These data also indicate that DW-11 will have significant activity as an anti-cancer agent, especially against leukemia, colon cancer, breast cancer, skin cancer and prostate cancer, as well as against skin conditions such as dry skin (lack of dermal hydration), undue skin slackness (insufficient skin firmness), insufficient sebum secretion and wrinkles.

DW-1 would also be expected to be very active in suppressing secondary hyperparathyroidism.

Calcium Mobilization from Bone in Vitamin D-Deficient Animals.

Using, vitamin D-deficient rats on a low calcium diet (0.02%), the activities of DW-1 and 1,25(OH)₂D₃ in intestine and bone were tested. As expected, the native hormone (1,25(OH)₂D₃) increased serum calcium levels at all dosages (FIG. 19). FIG. 19, however, also shows that DW-1 has little, if any, activity in mobilizing, calcium from bone. Administration of DW-1 at 7020 pmol/day for 4 consecutive days did not result in mobilization of bone calcium, and increasing the amount of DW-1 to 35,100 pmol/day was also without any substantial effect.

Intestinal Calcium Transport Activity.

FIG. 20 demonstrates that 1,25(OH)₂D₃ has significant activity in stimulating calcium transport in the gut, as expected. FIG. 20, however, also shows that DW-1 is about 50 times less potent than 1,25(OH)₂D₃ in stimulating calcium transport in the gut. Administration of DW-1 at 35,100 pmol/day, a dose that is 45 times greater than the 780 pmol/day dose tested for 1,25(OH)₂D₃, resulted in stimulating some intestinal calcium transport activity, but still not to the same extent as 1,25(OH)₂D₃ at 780 pmol/day.

These results illustrate that DW-1 is an excellent candidate for numerous human therapies as described herein, and that it may be particularly useful in a number of circumstances such as suppression of secondary hyperparathyroidism of renal osteodystrophy, autoimmune diseases, cancer, and psoriasis. DW-1 is an excellent candidate for treating psoriasis because: (1) it has significant transcription activity and cellular differentiation activity; (2) it is devoid of hypercalcemic liability unlike 1,25(OH)₂D₃; and (3) it is easily synthesized. Also, since DW-1 has significant binding activity to the vitamin D receptor, but has little ability to raise blood serum calcium, it may also be particularly useful for the treatment of secondary hyperparathyroidism of renal osteodystrophy.

These data also indicate that the compound DW-1 of the invention may be especially suited for treatment and prophylaxis of human disorders which are characterized by an imbalance in the immune system, e.g. in autoimmune diseases, including multiple sclerosis, lupus, diabetes mellitus, host versus graft rejection, and rejection of organ transplants; and additionally for the treatment of inflammatory diseases such as rheumatoid arthritis, asthma, and inflammatory bowel diseases such as celiac disease, ulcerative colitis and Crohn's disease. Acne, alopecia and hypertension are other conditions which may be treated with the compound DW-1 of the invention.

The compound DW-1 is also useful in preventing or treating obesity, inhibiting adipocyte differentiations, inhibiting SCD-1 gene transcription, and/or reducing body fat in animal subjects. Therefore in some embodiments, a method of preventing or treating obesity, inhibiting adipocyte differentiations, inhibiting SCD-1 gene transcription, and/or reducing body fat in an animal subject includes administering to the animal subject, an effective amount of DW-1 or a pharmaceutical composition that includes DW-1. Administration of the compound or the pharmaceutical compositions to the subject inhibits adipocyte differentiation, inhibits gene transcription, and/or reduces body fat in the animal subject.

### Example 9

### Biological Activity of TA-2

With regard to the 2α-methyl analog TA-2, the introduction of a methyl group in an alpha orientation to the 2-position and the removal of a methyl group at the 26 position in the side chain of (20S,25S)-19-nor-1α,25-dihydroxyvitamin D₃ had little 6 no effect on binding to the full length recombinant rat vitamin D receptor, as compared to 1α,25-dihydroxyvitamin D₃. The compound TA-2 bound equally well to the receptor as compared to the standard 1,25-(OH)₂D₃ (FIG. 21). It might be expected from these results that compound TA-2 would have equivalent biological activity. Surprisingly, however, compound TA-2 is a highly selective analog with unique biological activity.

FIG. 24 demonstrates that TA-2 is more than 50 times less potent than 1,25(OH)₂D₃ in bone, and thus has very little bone calcium mobilization activity, as compared to 1,25(OH)₂D₃. FIG. 25 demonstrates that TA-2 is approximately 1 log (10 times) less potent than 1,25(OH)₂D₃ in the intestine. Thus, TA-2 may be characterized as having little, if any, calcemic activity.

FIG. 22 illustrates that TA-2 is 4 times more potent than 1,25(OH)₂D₃ on HL-60 differentiation, making it an excellent candidate for the treatment of psoriasis and cancer, especially against leukemia, colon cancer, breast cancer, skin cancer and prostate cancer. In addition, due to its relatively high cell differentiation activity, this compound provides a therapeutic agent for the treatment of various skin conditions including wrinkles, lack of adequate dermal hydration, i.e. dry skin, lack of adequate skin firmness, i.e. slack skin, and insufficient sebum secretion. Use of this compound thus not only results in moisturizing of skin but also improves the barrier function of skin.

FIG. 23 illustrates that the compound TA-2 has more transcriptional activity than 1α,25-dihydroxyvitamin D₃ in bone cells, i.e. TA-2 is about 4-times more potent than 1,25(OH)₂D₃ in increasing transcription of the 24-hydroxylase gene. This result, together with the cell differentiation activity of FIG. 22, suggests that TA-2 will be very effective in psoriasis because it has direct cellular activity in causing cell differentiation and in suppressing cell growth. These data also indicate that TA-2 may have significant activity as an anti-cancer agent, especially against leukemia, colon cancer, breast cancer, skin cancer and prostate cancer.

The strong activity of TA-2 on HL-60 differentiation suggests it will be active in suppressing growth of parathyroid glands and in the suppression of the preproparathyroid gene.

### Interpretation of TA-2Data

VDR Binding HL-60 Cell Differentiation, and Transcription Activity.

TA-2 (Kᵢ=1×10⁻M) is equivalent to the natural hormone 1α,25-dihydroxyvitamin D₃ (Kᵢ=1×10⁻¹⁰M) in its ability to compete with [³H]— 1,25(OH)₂D₃ for binding to the full-length recombinant rat vitamin D receptor (FIG. 21). TA-2 is also about 4 times more potent (EC₅₀=5×10⁻¹⁰M) in its ability (efficacy or potency) to promote HL-60 differentiation as compared to 1α,25-dihydroxyvitamin D₃ (EC₅₀=2×10⁻⁹M) (See FIG. 22). Also, compound TA-2 (EC₅₀=8×10⁻¹¹M) has significant transcriptional activity in bone cells as compared to 1α,25-dihydroxyvitamin D₃ (EC₅₀=2×10⁻¹⁰M) (See FIG. 23), i.e. TA-2 is about 4 times more potent than 1,25(OH)₂D₃ in transcription activity.

These results suggest that TA-2 will be very effective in psoriasis because it has direct cellular activity in causing cell differentiation and in suppressing cell growth. These data also indicate that TA-2 will have significant activity as an anti-cancer agent, especially against leukemia, colon cancer, breast cancer, skin cancer and prostate cancer, as well as against skin conditions such as dry skin (lack of dermal hydration), undue skin slackness (insufficient skin firmness), insufficient sebum secretion and wrinkles.

TA-2 would also be expected to be very active in suppressing secondary hyperparathyroidism.

Calcium Mobilization from Bone in Vitamin D=Deficient Animals.

Using vitamin D-deficient rats on a low calcium diet (0.02%), the activities of TA-2 and 1,25(OH)₂D₃ in intestine and bone were tested. As expected, the native hormone (1,25(OH)₂D3) increased serum calcium levels at all dosages (FIG. 24). FIG. 24, however, also shows that TA-2 has little, if any, activity in mobilizing, calcium from bone. Administration of TA-2 at 7020 pmol/day for 4 consecutive days did not result in mobilization of bone calcium, and increasing the amount of TA-2 to 35,100 pmol/day was also without any substantial effect.

Intestinal Calcium Transport Activity.

FIG. 25 demonstrates that 1,25(OH)₂D₃ has significant activity in stimulating calcium transport in the gut, as expected. FIG. 25, however, also demonstrates that TA-2 is about 10 times (one log) less potent than 1,25(OH)₂D₃ in stimulating calcium transport in the gut. Administration of TA-2 at 7020 pmol/day, a dose that is 9 times greater than the 780 pmol/day dose tested for 1,25(OH)₂D₃, resulted in stimulating some intestinal calcium transport activity, but still not to the same extent as 1,25(OH)₂D₃ at 780 pmol/day.

These results illustrate that TA-2 is an excellent candidate for numerous human therapies as described herein, and that it may be particularly useful in a number of circumstances such as suppression of secondary hyperparathyroidism of renal osteodystrophy, autoimmune diseases, cancer, and psoriasis. TA-2 is an excellent candidate for treating psoriasis because: (1) it has significant transcription activity and cellular differentiation activity; (2) it is devoid of hypercalcemic liability unlike 1,25(OH)₂D₃, and (3) it is easily synthesized. Also, since TA-2 has significant binding activity to the vitamin D receptor but has little ability to raise blood serum calcium, it may also be particularly useful for the treatment of secondary hyperparathyroidism of renal osteodystrophy.

These data also indicate that the compound TA-2 of the invention may be especially suited for treatment and prophylaxis of human disorders which are characterized by an imbalance in the immune system, e.g. in autoimmune diseases, including multiple sclerosis, lupus, diabetes mellitus, host versus graft rejection, and rejection of organ transplants; and additionally for the treatment of inflammatory diseases such as rheumatoid arthritis, asthma, and inflammatory bowel diseases such as celiac disease, ulcerative colitis and Crohn's disease. Acne, alopecia and hypertension are other conditions which may be treated with the compound TA-2 of the invention.

The compound TA-2 is also useful in preventing or treating obesity, inhibiting adipocyte differentiations, inhibiting SCD-11 gene transcription, and/or reducing body fat in animal subjects. Therefore in some embodiments, a method of preventing or treating obesity, inhibiting adipocyte differentiations, inhibiting SCD-1 gene transcription, and/or reducing body fat in an animal subject includes administering to the animal subject an effective amount of TA-2 or a pharmaceutical composition that includes TA-2. Administration of the compound or the pharmaceutical compositions to the subject inhibits adipocyte differentiation inhibits gene transcription, and/or reduces body fat in the animal subject.

### Example 10

### Biological Activity of IB-1

With regard to the 2β-methyl analog IB-1, the introduction of a methyl group in a beta orientation to the 2-position and the removal of a methyl group at the 26 position in the side chain of (20S,25S)-19-nor-1α,25-dihydroxyvitamin D₃ reduced its ability to bind to the full length recombinant rat vitamin D receptor, as compared to α,25-dihydroxyvitamin D₃. The compound IB-1 exhibits 15 times lower activity in binding to the receptor as compared to the standard 1,25-(OH)₂D₃ (FIG. 26). It might be expected from these results that compound IB-1 would not have any desirable biological activity. Surprisingly, however, compound IB-1 is a highly selective analog with unique biological activity.

FIG. 29 demonstrates that IB-1 is more than 50 times less potent than 1,25(OH)₂D₃ in bone, and thus has very little bone calcium mobilization activity, as compared to 1,25(OH)₂D₃. FIG. 30 demonstrates that IB-1 is approximately 50 times less potent than 1,25(OH)₂D₃ in stimulating calcium transport in the gut. Thus, IB-1 may be characterized as having little, if any, calcemic activity.

FIG. 27 illustrates that IB-1 is only one log (10 times) less potent than 25(OH)₂D₃ on HL-60 differentiation, making it a candidate for the treatment of psoriasis and cancer, especially against leukemia, colon cancer, breast cancer, skin cancer and prostate cancer. In addition, due to its relatively high cell differentiation activity, this compound provides a therapeutic agent for the treatment of various skin conditions including wrinkles, lack of adequate dermal hydration, i.e. dry skin, lack of adequate skin firmness, i.e. slack skin, and insufficient sebum secretion. Use of this compound thus not only results in moisturizing of skin but also improves the barrier function of skin.

FIG. 28 illustrates that the compound IB-1 has about 3 logs less transcriptional activity than 1α,25-dihydroxyvitamin D₃ in bone cells; i.e. in bone cells, IB-1 is approximately 30 times less potent than 1,25(OH)₂D₃ in increasing transcription of the 24-hydroxylase gene. This result, together with the cell differentiation activity of FIG. 27, suggests that IB-1 will be very effective in psoriasis because it has direct cellular activity in causing cell differentiation and in suppressing cell growth. These data also indicate that IB-1 may have significant activity as an anti-cancer agent, especially against leukemia, colon cancer, breast cancer, skin cancer and prostate cancer.

The strong activity of IB-1 on HL-60 differentiation suggests it will be active in suppressing growth of parathyroid glands and in the suppression of the preproparathyroid gene.

### Interpretation of IB-1 Data

VDR Binding HL-60 Cell Differentiation, and Transcription Activity.

IB-1 (Kᵢ=8×10⁻¹⁰M) is nearly equivalent (i.e. only about 15 times less active) to the natural hormone 1α,25-dihydroxyvitamin D₃ (Kᵢ=5×10⁻¹⁰M) in its ability to compete with [³H]— 1,25(OH)₂D₃ for binding to the full-length recombinant rat vitamin D receptor (FIG. 26). IB-1 is also only about one log less potent (EC₅₀=2×10⁻⁸M) in its ability (efficacy or potency) to promote HL-60 differentiation as compared to 1α,25-dihydroxyvitamin D₃ (EC₅₀=2×10⁻⁹M) (See FIG. 27). Also, compound IB-1 (EC₅₀=7×10⁻⁹M) has significant transcriptional activity in bone cells (i.e. only about 3 logs less potent) as compared to 1α,25-dihydroxyvitamin D₃ (EC₅₀=2×10⁻¹⁰M) (See FIG. 28).

These results suggest that IB-1 will be very effective in psoriasis because it has direct cellular activity in causing cell differentiation and in suppressing cell growth. These data also indicate that IB-1 will have significant activity as an anti-cancer agent, especially against leukemia, colon cancer, breast cancer, skin cancer and prostate cancer, as well as against skin conditions such as dry skin (lack of dermal hydration), undue skin slackness (insufficient skin firmness), insufficient sebum secretion, and wrinkles.

IB-1 would also be expected to be very active in suppressing secondary hyperparathyroidism.

Calcium Mobilization from Bone in Vitamin D-Deficient Animals.

Using vitamin D-deficient rats on a low calcium diet (0.02%); the activities of IB-1 and 1,25(OH)₂D₃ in intestine and bone were tested. As expected, the native hormone (1,25(OH)₂D₃) increased serum calcium levels at all dosages (FIG. 29). FIG. 29, however, also shows that IB-1 has little, if any, activity in mobilizing calcium from bone. Administration of IB-1 at 35,100 pmol/day for 4 consecutive days did not result in mobilization of bone calcium.

Intestinal Calcium Transport Activity.

FIG. 30 demonstrates that 1,25(OH)₂D₃ has significant activity in stimulating calcium transport in the gut, as expected. FIG. 30, however, also demonstrates that IB-1 is about 50 times less potent than 1,25(OH)₂D₃ in stimulating calcium transport in the gut. Administration of B-1 at 35,100 pmol/day, a dose that is 45 times greater than the 780 pmol/day dose tested for 1,25(OH)₂D₃, resulted in stimulating some intestinal calcium transport activity; but still not to the same extent as 1,25(OH)₂D₃ at 780 pmol/day.

These results illustrate that IB-1 is an excellent candidate for numerous human therapies as described herein, and that it may be particularly useful in a number of circumstances such as suppression of secondary hyperparathyroidism of renal osteodystrophy, autoimmune diseases, cancer, and psoriasis. IB-1 is an excellent candidate for treating psoriasis because: (1) it has significant transcription activity and cellular differentiation activity; (2) it is devoid of hypercalcemic liability unlike 1,25(OH)₂D₃; and (3) it is easily synthesized. Also, since IB-1 has significant binding activity to the vitamin D receptor, but has little ability to raise blood serum calcium, it may also be particularly useful for the treatment of secondary hyperparathyroidism of renal osteodystrophy.

These data also indicate that the compound IB-1 of the invention may be especially suited for treatment and prophylaxis of human disorders which are characterized by an imbalance in the immune system, e.g. in autoimmune diseases, including multiple sclerosis, lupus, diabetes mellitus, host versus graft rejection, and rejection of organ transplants and additionally for the treatment of inflammatory diseases such as rheumatoid arthritis, asthma, and inflammatory bowel diseases such as celiac disease, ulcerative colitis and Crohn's disease. Acne, alopecia and hypertension are other conditions which may be treated with the compound IB-1 of the invention.

The compound IB-1 is also useful in preventing or treating obesity, inhibiting adipocyte differentiations, inhibiting SCD-1 gene transcription, and/or reducing body fat in animal subjects. Therefore in some, embodiments, a method of preventing or treating obesity, inhibiting adipocyte differentiations, inhibiting SCD-1 gene transcription, and/or reducing body fat in an animal subject includes administering to the animal subject, an effective amount of IB-1 or a pharmaceutical composition that includes IB-1. Administration of the compound or the pharmaceutical compositions to the subject inhibits adipocyte differentiation, inhibits gene transcription, and/or reduces body fat in the animal subject.

### Example 11

### Biological Activity of NC-2

With regard to the 2α-methyl analog NC-2, the introduction of a methyl group in an alpha orientation to the 2-position and the removal of a methyl group at the 26 position in the side chain of (20R,25S)-19-nor-1α,25-dihydroxyvitamin D₃ had little or no effect on binding to the full length recombinant rat vitamin D receptor, as compared to 1α,25-dihydroxyvitamin D₃. The compound NC-2 bound equally well to the receptor as compared to the standard 1,25-(OH)₂D₃ (FIG. 31). It might be expected from these results that compound NC-2 would have equivalent biological activity. Surprisingly, however, compound NC-2 is a highly selective analog with unique biological activity.

FIG. 34 demonstrates that NC-2 is more than 50 times less potent than 1,25(OH)₂D₃ in bone, and thus has very little bone calcium mobilization activity, as compared to 1,25(OH)₂D₃. FIG. 35 demonstrates that NC-2 is approximately 5 logs (50 times less potent than 1,25(OH)₂D₃ in the intestine. Thus, NC-2 may be characterized as having little, if any, calcemic activity.

FIG. 32 illustrates that NC-2 is 4 times more potent than 1,25(OH)₂D₃ on HL-60 differentiation, making it an excellent candidate for the treatment of psoriasis and cancer, especially against leukemia, colon cancer, breast cancer, skin cancer and prostate cancer. In addition, due to its relatively high cell differentiation activity, this compound provides a therapeutic agent for the treatment of various skin conditions including wrinkles, lack of adequate dermal hydration, i.e. dry skin, lack of adequate skin firmness, i.e. slack skin, and insufficient sebum secretion. Use of this compound thus hit only results in moisturizing of skin but also improves the barrier function of skin.

FIG. 33 illustrates that the compound NC-2 has slightly less transcriptional activity than 1α,25-dihydroxyvitamin D₃ in bone cells, i.e. NC-2 is only about one-half log less potent than 1,25(OH)₂D₃ in increasing transcription of the 24-hydroxylase gene. This result, together with the cell differentiation activity of FIG. 32, suggests that NC-2 will be very effective in psoriasis because it has direct cellular activity in causing cell differentiation and in suppressing cell growth. These data also indicate that NC-2 may have significant activity as an anti-cancer agent, especially against leukemia, colon cancer, breast cancer, skin cancer and prostate cancer.

The strong activity of NC-2 on HL-60 differentiation suggests it will be active in suppressing growth of parathyroid glands and in the suppression of the preproparathyroid gene.

### Interpretation of NC-2Data

VDR Binding HL-60 Cell Differentiation, and Transcription Activity.

NC-2 (Kᵢ=2×10⁻¹⁰M) is equivalent to the natural hormone 1α,25-dihydroxyvitamin D₃ (Kᵢ=1×10⁻¹⁰M) in its ability to compete with [³H]— 1,25(OH)₂D₃ for binding to the full-length recombinant rat vitamin D receptor (FIG. 31). There is also little difference between NC-2 (EC₅₀=5×10⁻⁹M) in its ability (efficacy or potency) to promote HL-60 differentiation as compared to 1α,25-dihydroxyvitamin D₃ (EC₅₀=2×10⁻⁹M) (See FIG. 32). Also, compound NC-2 (EC₅₀=6×10⁻¹⁰M) has significant transcriptional activity in bone cells as compared to 1α,25-dihydroxyvitamin D₃ (EC₅₀=2×10⁻¹⁰M) (See FIG. 33.

These results suggest that NC-2 will be very effective in psoriasis because it has direct cellular activity in causing cell differentiation and in suppressing cell growth. These data also indicate that NC-2 will have significant activity as an anti-cancer agent, especially against leukemia, colon cancer, breast cancer, skin cancer and prostate cancer, as well as against skin conditions such as dry skin (lack of dermal hydration), undue skin slackness (insufficient skin firmness), insufficient sebum secretion and wrinkles.

NC-2 would also be expected to be very active in suppressing secondary hyperparathyroidism.

Calcium Mobilization from Bone in Vitamin D-Deficient Animals.

Using vitamin D-deficient rats on a low calcium diet (0.02%), the activities of NC-2 and 1,25(OH)₂D₃ in intestine and bone were tested. As expected, the native hormone (1,25(OH)₂D₃) increased serum calcium levels at all dosages (FIG. 34). FIG. 34, however, also shows that NC-2 has little, if any, activity in mobilizing, calcium from bone. Administration of NC-2 at 7020 pmol/day for 4 consecutive days did not result, in mobilization of bone calcium, and increasing the amount of NC-2 to 35,100 pmol/day was also without any substantial effect.

Intestinal Calcium Transport Activity.

FIG. 35 demonstrates that 1,25(OH)₂D₃ has significant activity in stimulating calcium transport in the gut, as expected. FIG. 35, however, also demonstrates that NC-2 is about 50 times less potent than 1,25(OH)₂D₃ in stimulating calcium transport in the gut. Administration of NC-2 at 7020 pmol/day, a dose that is 9 times greater than the 780 pmol/day dose tested for 1,25(OH)₂D₃, and then increasing the amount of NC-2 to 35,100 pmol/day, a dose that is 45 times greater than the 780 pmol/day dose tested for 1,25(OH)₂D₃, resulted in stimulating some intestinal calcium transport activity, but still not to the same extent as 1,25(OH)₂D₃ at 780 pmol/day.

These results illustrate that NC-2 is an excellent candidate for numerous human therapies as described herein, and that it may be particularly useful in a number of circumstances such as suppression of secondary hyperparathyroidism of renal osteodystrophy, autoimmune diseases, cancer, and psoriasis. NC-2 is an excellent candidate for treating psoriasis because: (1) it has significant transcription activity and cellular differentiation activity, (2) it is devoid of hypercalcemic liability unlike 1,25(OH)₂D₃; and (3) it is easily synthesized. Also, since NC-2 has significant binding activity to the vitamin D receptor, but has little ability to raise blood serum calcium, it may also be particularly useful for the treatment of secondary hyperparathyroidism of renal osteodystrophy.

These data also indicate that the compound NC-2 of the invention may be especially suited for treatment and prophylaxis of human disorders which are characterized by an imbalance in the immune system, e.g. in autoimmune diseases, including multiple sclerosis, lupus, diabetes mellitus, host versus graft rejection, and rejection of organ transplants; and additionally for the treatment of inflammatory diseases such as rheumatoid arthritis, asthma, and inflammatory bowel diseases such as celiac disease, ulcerative colitis and Crohn's disease. Acne, alopecia and hypertension are other conditions which may be treated with the compound NC-2 of the invention.

The compound NC-2 is also useful in preventing or treating obesity, inhibiting adipocyte differentiations, inhibiting SCD-1 gene transcriptional, and/or reducing body fat in animal subjects. Therefore in some embodiments, a method of preventing or treating obesity, inhibiting adipocyte differentiations, inhibiting SCD-1 gene transcription, and/or reducing body fat in an animal subject includes administering to the animal subject, an effective amount of NC-2 or a pharmaceutical composition that includes NC-2. Administration of the compound or the pharmaceutical compositions to the subject inhibits adipocyte differentiation, inhibits gene transcription, and/or reduces body fat in the animal subject.

### Example 12

### Biological Activity of TH-1

With regard to the 2β-methyl analog TH-1, the introduction of a methyl group in a beta orientation to the 2-position and the removal of a methyl group at the 26 position in the side chain of (20R,25S)-19-nor-1α,25-dihydroxyvitamin D₃ reduced its ability to bind to the full length recombinant rat vitamin D receptor, as compared to 1α,25-dihydroxyvitamin D₃. The compound TH-1 exhibits 50 times lower activity in binding to the receptor as compared to the standard 1,25-(OH)₂D₃ (FIG. 36). It might be expected from these results that compound TH-1 would not have any desirable biological activity. Surprisingly, however, compound TH-1 is a highly selective analog with unique biological activity.

FIG. 39 demonstrates that TH-1 is more than 50 times less potent than 1,25(OH)₂D₃ in bone, and thus has very little bone calcium mobilization activity, as compared to 1,25(OH)₂D₃. FIG. 40 demonstrates that TH-1 is approximately 50 times less potent than 1,25(OH)₂D₃ in stimulating calcium transport in the gut. Thus, TH-1 may be characterized as having little, if any, calcemic activity.

FIG. 37 illustrates that TH-1 is only 3 logs (30 times) less potent than 1,25(OH)₂D₃ on HL-60 differentiation, making it a candidate for the treatment of psoriasis and cancer, especially against leukemia, colon cancer, breast cancer, skin cancer and prostate cancer. In addition, due to its relatively high cell differentiation activity, this compound provides a therapeutic agent for the treatment of various skin conditions including wrinkles, lack of adequate dermal hydration, i.e. dry skin, lack of adequate skin firmness, i.e. slack skin, and insufficient sebum secretion. Use of this compound thus not only results in moisturizing of skin but also improves the barrier function of skin.

FIG. 38 illustrates that the compound TH-1 is only about 2 logs less potent in transcriptional activity than 1α,25-dihydroxyvitamin D₃ in bone cells, i.e. in bone cells, TH-1 is nearly 20 times less potent than 1,25(OH)₂D₃ in increasing transcription of the 24-hydroxylase gene. This result, together with the cell differentiation activity of FIG. 37, suggests that TH-1 will be very effective in psoriasis because it has direct cellular activity in causing cell differentiation and in suppressing cell growth. These data also indicate that TH-1 may have significant activity as an anti-cancer agent, especially against leukemia, colon cancer breast cancer, skin cancer and prostate cancer.

The strong activity of TH-1 on HL-60 differentiation suggests it will be active in suppressing growth of parathyroid glands and in the suppression of the preproparathyroid gene.

### Interpretation of TH-1 Data

VDR Binding, HL-60 Cell Differentiation, and Transcription Activity.

TH-1 (Kᵢ=5×10⁻⁹M) is 50 times less potent than the natural hormone 1α,25-dihydroxyvitamin D₃ (Kᵢ=1×10⁻¹⁰M) in its ability to compete with [³H]— 1,25(OH)₂D₃ for binding to the full-length recombinant rat vitamin D receptor (FIG. 36). TH-1 is also about 30 times less potent (EC₅₀=7×10⁻⁸M) in its ability (efficacy or potency) to promote HL-60 differentiation as compared to 1α,25-dihydroxyvitamin D₃ (EC₅₀=2×10⁻⁹M) (See FIG. 37). Also, compound TH-1 (EC₅₀=3×¹⁰⁻⁸M) has significant transcriptional activity in bone cells (i.e. only about 2 logs less potent) as compared to 1α,25-dihydroxyvitamin D₃ (EC₅₀=2×10⁻¹⁰M) (See FIG. 38).

These results suggest that TH-1 will be very effective in psoriasis because it has direct cellular activity in causing cell differentiation and in suppressing cell growth. These data also indicate that TH-1 will have significant activity as a anti-cancer agent, especially against leukemia, colon cancer, breast cancer, skin cancer and prostate cancer, as well as against skin conditions such as dry skin (lack of dermal hydration), undue skin slackness (insufficient skin firmness), insufficient sebum secretion and wrinkles.

TH-1 would also be expected to be very active in suppressing secondary hyperparathyroidism.

Calcium Mobilization from Bone in Vitamin D-Deficient Animals.

Using vitamin D-deficient rats on a low calcium diet (0.02%), the activities of TH-1 and 1,25(OH)₂D₃ in intestine and bone were tested. As expected, the native hormone (1,25 (OH)₂D₃) increased serum calcium levels at all dosages (FIG. 39). FIG. 39, however, also shows that TH-1 has little, if any, activity in mobilizing calcium from bone. Administration of TH-1 at 35,100 pmol/day for 4 consecutive days did not result in mobilization of bone calcium.

Intestinal Calcium Transport Activity.

FIG. 40 demonstrates that 1,25(OH)₂D3 has significant activity in stimulating calcium transport in the gut, as expected. FIG. 40, however, also shows that TH-1 is about 50 times less potent than 1,25(OH)₂D₃ in stimulating calcium transport in the gut. Administration of TH-1 at 35,100 pmol/day, a dose that is 45 times greater than the 780 pmol/day dose tested for 1,25(OH)₂D₃, resulted in stimulating some intestinal calcium transport activity, but still not to the same extent as 1,25(OH)D₃ at 780 pmol/day.

These results illustrate that TH-1 is an excellent candidate for numerous human therapies as described herein, and that it may be particularly useful in a number of circumstances such as suppression of secondary hyperparathyroidism of renal osteodystrophy, autoimmune diseases, cancer, and psoriasis. TH-1 is an excellent candidate for treating psoriasis because: (1) it has significant transcription activity and cellular differentiation activity; (2) it is devoid of hypercalcemic liability unlike 1,25(OH)₂ D₃; and (3) it is easily synthesized. Also, since TH-1 has significant binding activity to the vitamin D receptor, but has little ability to, raise blood serum calcium, it may also be particularly useful for the treatment of secondary hyperparathyroidism of renal osteodystrophy.

These data also indicate that the compound TH-1 of the invention may be especially suited for treatment and prophylaxis of human disorders which are characterized by an imbalance in the immune system, e.g. in autoimmune diseases, including multiple sclerosis, lupus, diabetes mellitus, host versus graft rejection, and rejection of organ transplants and additionally for the treatment of inflammatory diseases such as rheumatoid arthritis, asthma, and inflammatory bowel diseases such as celiac disease, ulcerative colitis and Crohn's disease. Acne, alopecia and hypertension are other conditions which may be treated with the compound TH-1 of the invention.

The compound TH-1 is also useful in preventing or treating obesity, inhibiting adipocyte differentiations, inhibiting SCD-1 gene transcription, and/or reducing body fat in animal subjects. Therefore in some embodiments, a method of preventing or treating obesity, inhibiting adipocyte differentiations, inhibiting SCD-1 gene transcription, and/or reducing body fat in an, animal subject includes administering to the animal subject, an effective amount of TH-1 or a pharmaceutical composition that includes TH-1. Administration of the compound or the pharmaceutical compositions to the subject inhibits adipocyte differentiation, inhibits gene transcription, and/or reduces body fat in the animal subject.

For treatment purposes, the compounds of this invention defined by formula I may be formulated for pharmaceutical applications as a solution innocuous solvents, or as an emulsion, suspension or dispersion in suitable solvents or carriers, or as pills, tablets or capsules, together with solid carriers, according to conventional methods known in the art. Any such formulations may also contain other pharmaceutically-acceptable and non-toxic excipients such as stabilizers, anti-oxidants, binders, coloring; agents or emulsifying or taste-modifying agents.

The compounds of formula I may be administered orally topically, parenterally, nasally, rectally, sublingually or transdermally. The compounds may be advantageously administered by injection or by intravenous infusion or suitable sterile solutions, or in the form of liquid or solid doses via the alimentary canal, or in the form of creams, ointments, patches, or similar vehicles suitable for transdermal applications. A dose of from 0.01 μg to 1000 μg per day of compounds I, preferably from about 0.1 μg to about 500 μg per day, is appropriate for prevention and/or treatment purposes, such dose being adjusted according to the disease to be treated, its severity and the response of the subject as is well understood in the art. Since the compounds exhibit specificity of action, each may be suitably administered alone, or together with graded doses of another active vitamin D compound—e.g. 1α-hydroxyvitamin D₂ or D₃, or 1α,25-dihydroxyvitamin D₃—in situations where different degrees of bone mineral mobilization and calcium transport stimulation is found to be advantageous.

Compositions for use in the above-mentioned treatments comprise an effective amount of compounds I, as further defined by the above formula Ia and Ib, as the active ingredient, and a suitable carrier. An effective amount of such compounds for use in accordance with this invention is from about 0.01 μg to about 100 μg per gm of composition, preferably from about 0.1 μg to about 50 μg per gram of composition, and may be formulated to be administered topically, transdermally, orally, nasally, rectally, sublingually or parenterally in dosages of from about 0.01 μg/day to about 1000 μg/day, and preferably from about 0.1 μg/day to about 500 μg/day.

The compounds I may be formulated as creams, lotions, ointments, topical patches, pills, capsules or tablets, or in liquid form as solutions, emulsions, dispersions, or suspensions in pharmaceutically innocuous and acceptable solvent or oils, and such preparations may contain in addition other pharmaceutically innocuous or beneficial components, such as stabilizers, antioxidants, emulsifiers, coloring agents, binders or taste-modifying agents.

The compounds I may be advantageously administered in amounts sufficient to effect the differentiation of promyelocytes to normal macrophages. Dosages as described above are suitable, it being understood that the amounts given are to be adjusted in accordance with the severity of the disease, and the condition and response of the subject as is well understood in the art.

The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof.

Formulations of the present invention suitable for oral administration may be in the form of discrete units as capsules, sachets, tablets or lozenges, each containing a predetermined amount of the active ingredient; in the form of a powder or granules, in the form of a solution or a suspension in an aqueous liquid or non-aqueous liquid; or in the form of an oil-in-water emulsion or a water-in-oil emulsion.

Formulations for rectal administration may be in the form of a suppository incorporating the active ingredient and carrier such as cocoa butter, or in the form of an enema.

Formulations suitable for parenteral administration conveniently comprise a sterile oily or aqueous preparation of the active ingredient which is preferably isotonic with the blood of the recipient.

Formulations suitable for topical administration include liquid or semi liquid preparations such as liniments, lotions, applicants, oil-in-water or water-in-oil emulsions such as creams, ointments or pastes; or solutions or suspensions such as drops; or as sprays.

For nasal administration, inhalation of powder, self-propelling or spray formulations, dispensed with a spray can, a nebulizer or an atomizer can be used. The formulations, when dispensed, preferably have a particle size in the range of 10 to 100μ.

The formulations may conveniently be presented in dosage unit form and may be prepared by any of the methods well known in the art of pharmacy. By the term “dosage unit” is meant a unitary, i.e. a single dose which is capable of being administered to a patient as a physically and chemically stable unit dose comprising either the active ingredient as such or a mixture of it with solid or liquid pharmaceutical diluents or carriers.

We claim:

1. A compound having the formula: 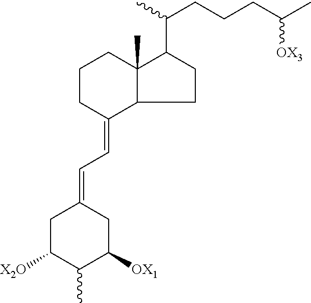
wherein the methyl group attached to carbon 2 may have an R or S configuration, and where the methyl group attached to carbon 20 may have an R or S configuration, and where the substituent —OX₃ may have an R or S configuration, and where X₁, X₂ and X₃, which may be the same or different, are each independently selected from hydrogen or a hydroxy-protecting group.

2. The compound of claim 1 wherein X₂ is hydrogen.

3. The compound of claim 1 wherein X₁, X₂ and X₃ are each hydrogen.

4. The compound of claim 1 wherein X₁, X₂ and X₃ are each t-butyldimethylsilyl.

5. A pharmaceutical composition containing an effective amount of at least one compound as claim in claim 1 together with a pharmaceutically acceptable excipient.

6. The pharmaceutical composition of claim 5 wherein said effective amount comprises from about 0.01 μg to about 100 μg per gram of composition.

7. A compound according to claim 1 having the formula: 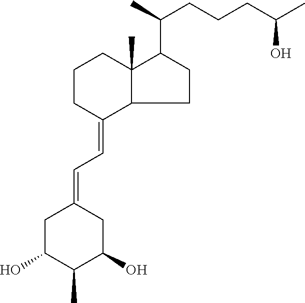
and the name (20S,25R)-2α-methyl-19,26-dinor-1α,25-dihydroxyvitamin D₃.

8. A compound accordingly to claim 1 having the formula: 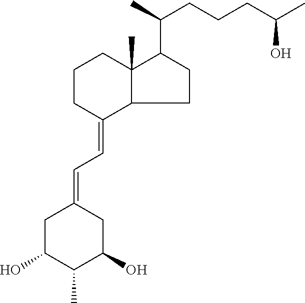
and the name (20S,25R)-2β-methyl-19,26-dinor-1α,25-dihydroxyvitamin D₃.

9. A compound according to claim 1 having the formula: 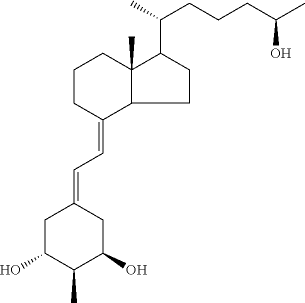
and the name (20R,25R)-2α-methyl-19,26-dinor-1α,25-dihydroxyvitamin D₃.

10. A compound according to claim 1 having the formula: 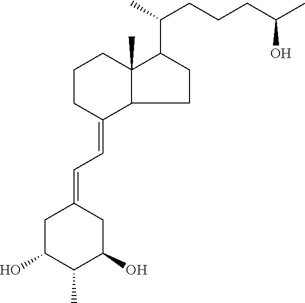
and the name (20R,25R)-2β-methyl-19,26-dinor-1α,25-dihydroxyvitamin D₃.

11. A compound according to claim 1 having the formula: 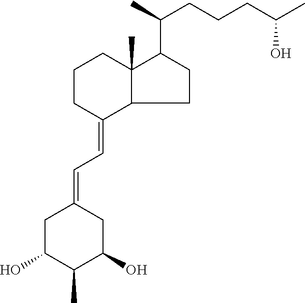
and the name (20S,25S)-2α-methyl-19,26-dinor-1α,25-dihydroxyvitamin D₃.

12. A compound according to claim 1 having the formula: 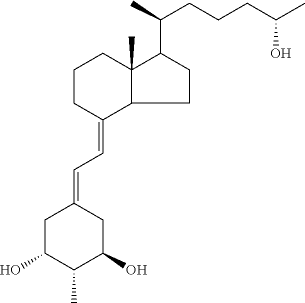
and the name (20S,25S)-2β-methyl-19,26-dinor-1α,25-dihydroxyvitamin D₃.

13. A compound according to claim 1 having the formula: 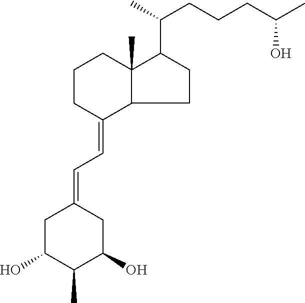
and the name (20R,25S)-2α-methyl-19,26-dinor-1α,25-dihydroxyvitamin D₃.

14. A compound according to claim 1 having the formula: 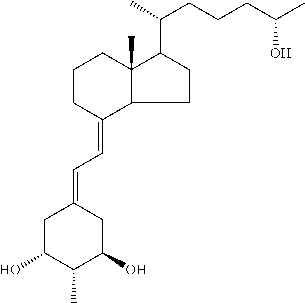
and the name (20R,25S)-2β-methyl-19,26-dinor-1α,25-dihydroxyvitamin D₃.

15. A method of treating a condition selected from psoriasis a cancerous disease selected from the group consisting of leukemia, colon cancer, breast cancer, skin cancer or prostate cancer; an autoimmune disease selected from the group consisting of multiple sclerosis, lupus, diabetes mellitus, host versus graft rejection, and rejection of organ transplants; an inflammatory disease selected from the group consisting of rheumatoid arthritis, asthma, and inflammatory bowel diseases; a skin disorder selected from the group consisting of wrinkles, lack of adequate skin firmness, lack of adequate dermal hydration and insufficient sebum secretion; renal osteodystrophy; and secondary hyperparathyroidism; comprising administering to a patient with said condition an effective amount of a compound having the formula: 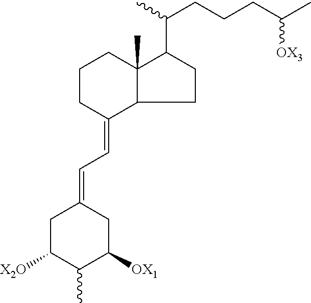
where the methyl group attached to carbon 2 may have an R or S configuration, and where the ethyl group attached to carbon 20 may have an R or S configuration and where the substituent —OX₃ may have an R or S configuration, and where X₁, X₂ and X₃, which may be the same or different, are each independently selected from hydrogen or a hydroxy-protecting group.

16. The method of claim 15 wherein the compound is adminstered orally, parenterally, transdermally, nasally, rectally, or sublingually.

17. The method of claim 15 wherein the condition is psoriasis, wrinkles, lack of adequate skin firmness, lack of adequate dermal hydration, or insufficient sebum secretion, and the compound is administered topically.

18. The method of claim 15 wherein the compound is to be administered in a dosage of from about 0.01 μg/day to about 1000 μg/day.

19. The method of claim 15 wherein the compound has the formula: 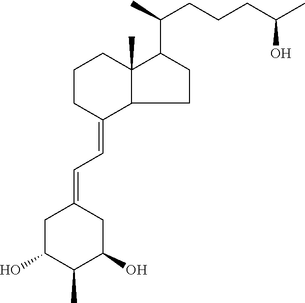
and the name (20S,25R)-2α-methyl-19,26-dinor-1α,25-dihydroxyvitamin D₃.

20. The method of claim 15 wherein the compound has the formula: 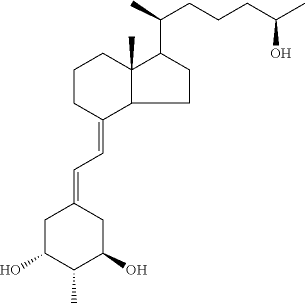
and the name (20S,25R)-2β-methyl-19,26-dinor-1α,25-dihydroxyvitamin D₃.

21. The method of claim 15 wherein the compound has the formula: 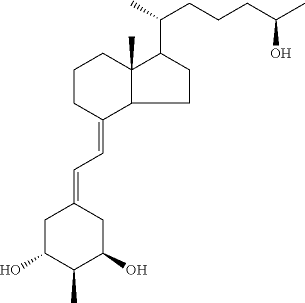
and the name (20R,25R)-2α-methyl-19,26-dinor-1α,25-dihydroxyvitamin D₃.

22. The method of claim 15 wherein the compound has the formula: 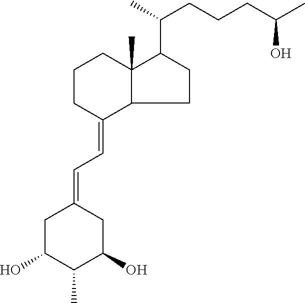
and the name (20R,25R)-2β-methyl-19,26-dinor-1α,25-dihydroxyvitamin D₃.

23. The method of claim 15 wherein the compound has the formula: 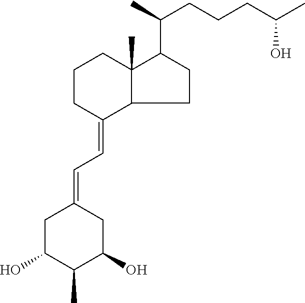
and the name (20S,25S)-2α-methyl-9,26-dinor-1α,25-dihydroxyvitamin D₃.

24. The method of claim 15 wherein the compound has the formula: 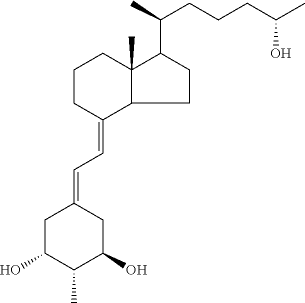
and the name (20S,25S)-2β-methyl-19,26-dinor-1α,25-dihydroxyvitamin D₃.

25. The method of claim 15 wherein the compound has the formula: 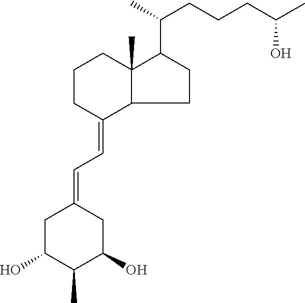
and the name (20R,25S)-2α-methyl-19,26-dinor-1α,25-dihydroxyvitamin D₃.

26. The method of claim 15 wherein the compound has the formula: 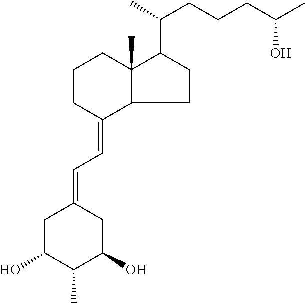
and the name (20R,25S)-2β-methyl-19,26-dinor-1α,25-dihydroxyvitamin D₃.
